SEQUENCE LISTING (712 sequences)

SEQ ID NO: 1 - protein, 847 aa, Artificial sequence
  Synthetic
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 2 - protein, 668 aa, Artificial sequence
  Synthetic
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 3 - protein, 406 aa, Artificial sequence
  Synthetic
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTV

SEQ ID NO: 4 - protein, 274 aa, Artificial sequence
  Synthetic
DVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 5 - protein, 453 aa, Artificial sequence
  Synthetic
EVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial sequence
  Synthetic
DIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 454 aa, Artificial sequence
  Synthetic
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial sequence
  Synthetic
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 848 aa, Artificial sequence
  Synthetic
MHLLGPWLLLLVLEYLAFSDSSKWNIEHPGTIYAWEGACVWVPCTYRVLDGALETFILFHNPEYNQNMSKFEGTRLYESTKDGKLPSGQKRVQFLGNKINNNCTLSIHPVHVNDSGQLGLRMVSKTEKWMERIHLNVSERPFPPRIQLPPKLQESQEVTLTCLLNFSCYGYQIQLQWLLEGVPMRQAAVTLTSLSTKSVFTRSELKFSPQWSHHGKIVTCELHDVDGKVLSEDMVQLNVKHTPKLTIEVTPNETTVRKGDSVTMTCKVNSSNPEYTTVSWLKDGIPLKEQNTLMLTLHKVTKSQSGRYCCRVSNDVGPATSEKVFLQVQYAPESSRVQISQSPAVEGSEVNFLCISPANPLPTNYTWYHNGKEVQGRTEKQFQIQKILPWHAGTYSCEAENILGIGERGPGTELDVQYPPKKVTMVIENPTPIREGDTVTLSCNYSSSNPIVNHYEWRPRGAWEEPSLGVLKIQNIGWNNTAVACAACNNWCSWASPVTLNVLYAPRGVRVRKIKPLSEIHSGNSVSLQCDFSSSHPKEVQFFWEKNGSLLGKESQLNFDSISPEDAGSYSCWVNNSIGQTASKAWTLEVLYAPRRLRVSMSQGNQVMEGKTATLICESDANPPVYSYAWFDWNNQSLPYSGRMLRLEPVKVQHSGAYWCQGTNRVGKGHSPLITLTVYYSPQTIGRRVAVGLGSCLAILILAMCGFKVQRRWKRTQSQQGLQENSSGQSFFVRNKKVRRTPLSEGPHSLGCYNPMMEDGISYATLRFPETNTPRTGDAETSELQRPPPDCDDTVTYSVLQKRQVGDYENVIPDFPEDEGIHYSELIQFGFGERPQAQENVDYVIVKH

SEQ ID NO: 10 - protein, 330 aa, Artificial sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 107 aa, Artificial sequence [sequence omitted]

SEQ ID NO: 12 - protein, 106 aa, Artificial sequence
  Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 113 aa, Artificial sequence
  Synthetic
QVQLQQPGAELVKPGTSVKVSCKASGYTFTNYWMHWVKQRPGQGLEWIGRIHPSDSDTEYNLQFKDKAALTVDKSSSTAYMRLSSLTSEDSAVYYCAMQFDYWGQGTTLTVSS

SEQ ID NO: 14 - protein, 107 aa, Artificial sequence
  Synthetic
DIKLTQSPSSMYASLGERVTITCKASQDINTFLSWFQQKPGKSPKILIYRANRLVDGVPSRFSGSGSGQDYSLTISSLESEDLGIYHCLQYDEFPRTFGGGTKVEFK

SEQ ID NO: 15 - protein, 115 aa, Artificial sequence
  Synthetic
QVQLQQPGTELVKPGASVKLSCKASGYTFTSFWMHWVKQRPGQGLEWIGNIDPSNGDTNYNEKFKSKATLTVDKSSSTAYIQLSSLTSEDSAVYYCASSYAMDYWGQGTSVTVSS

SEQ ID NO: 16 - protein, 106 aa, Artificial sequence
  Synthetic
DIKMTQSPSSMSASLGERVTITCKASQDINRFLSWFQQKPGKSPKILIYRANRLVDGVPSRFSGSGSGQDYSLTISSLEYEDMGIYYCLQYDEFRTFGGGTKVEIK

SEQ ID NO: 17 - protein, 113 aa, Artificial sequence
  Synthetic
QVQLQQPGTELVKPGASVKLSCKASGYTFTSYWMDWVKQRPGQGLEWIGNINPSNGGTNYNEKFKTKATLTVDKSSSTAYMQLSSLTPEDSAVYYCAGQLDYWGQGTTLTVSS

SEQ ID NO: 18 - protein, 107 aa, Artificial sequence
  Synthetic
DIKMTQSPSSMYASLGERVTITCKASQDINSYLSWFQQKPGKSPKILIYRANRLVDGVPSRFSGSGSGQDYSFTISSLEYEDMEIYYCLQYNEFPRTFGGGTKLEIK

SEQ ID NO: 19 - protein, 119 aa, Artificial sequence
  Synthetic
QVQLQQPGAELVKPGASVKLSCKTSGYTFTIYWMQWVKQRPGRGLEWIGRIDPHSGDTHYNENFTTKVTLTVDETSNTAYMHLSGLTSEDSAVYYCARRDYSYYVLDYWGQGTSVTVSS

SEQ ID NO: 20 - protein, 112 aa, Artificial sequence
  Synthetic
DVVLTQTPLSLPVSLGDQASISCRSSQSLVHINGDTYLHWYLQKPGQSPKLLIYTVFNRFSGVPDRFSGSGSGTDFTLKISRVEAEDLGAYFCSQSTHVPYTFGGGTKLEIK

SEQ ID NO: 21 - protein, 113 aa, Artificial sequence
  Synthetic
QVQLQQPGSELVRPGASVKLSCKASGYTFTNFWMHWVKQGHGQGLEWIGNIYPGSGDTYYNEKFRSRGSLTVDTSSSTAYMHLNSLTSEDSAVYYCTNQMDYWGQGTSVTVSS

SEQ ID NO: 22 - protein, 107 aa, Artificial sequence
  Synthetic
DIKMTQSPSSMYVSLGERVTISCTASQDTNRYLKWFHQKPGKSPKPLIYRTNRLIDGVPSRFSGTGSGQDYSLTVSNLEYEDMGFYYCLQYAEFPYTFGGGTKLEIK

SEQ ID NO: 23 - protein, 113 aa, Artificial sequence
  Synthetic
QVQLQQSGAELARPGASVKMSCRASGYTFTTYTMHWVKQRPGQGLEWIGYINPSSGYTQYNQKFKDKATLTADKSSSTAYMQLSSLTSEDSAVYYFTLQLDYWGQGTTLTVSS

SEQ ID NO: 24 - protein, 106 aa, Artificial sequence
  Synthetic
DIKMTQSPSSMYTSLGERVTITCKASQDINSYLSWFQQKPGKSPKILIYRANRLVDGVPSRFSGRGSGQDYSLTISSLEYEDMAIYYCLQYYELYTFGGGTKLEIK

SEQ ID NO: 25 - protein, 117 aa, Artificial sequence
  Synthetic
QVQLQQPGAELVKPGASVKMSCKASGYTFTSYWISWVKQRPGQGLEWIGDIYPGTGSSNYNEKFKSKATLTVDTSSSTAYMQLSSLTSEDSAVYYCARLKFEGIGYWGQGTTLTVSS

SEQ ID NO: 26 - protein, 106 aa, Artificial sequence
  Synthetic
QIVLTQSPAIMSASLGEEITLTCSASSSVSYMHWFQQKSGTSPKLLIYSISNLASGVPSRFSGSGSGTFYSLTISSVEAEDAADYYCHQWSSYPWTFGGGTKLEIK

SEQ ID NO: 27 - protein, 120 aa, Artificial sequence
  Synthetic
QVQLQQSGPELARPGASVKLSCKASGYTFTSYGISWVKQRTGQDLEWIGEIYPKLGTTYYNEKFKDKATLTTDRSSSTAYMALRSLTSEDSAVYFCACPHYYATRGGDYWGQGTTLTVSS

SEQ ID NO: 28 - protein, 107 aa, Artificial sequence
  Synthetic
EIVLTQSPATLSVTPGDSVTFSCRASQSISNHLHWYQQKSHESPRLLIRYASQSISGIPSRFSGSGSGTDFTLSLSSVETEDFGVYFCQQSHSWPYTFGGGTKLEIA

SEQ ID NO: 29 - protein, 113 aa, Artificial sequence
  Synthetic
EVQLQQSGPVLVKPGASVKMSCKASGYTFSDYFMNWVKQSHGKSLEWIGIINSYSGGTSYNQKFKGKATLTVDKSSSTAYMELNSLTSEDSAVYYCARWMDYWGQGTSVTVSS

SEQ ID NO: 30 - protein, 107 aa, Artificial sequence
  Synthetic
DVQITQSPSYLAASPGETITINCRTSESISKHLAWYQEKPGKTNKLLIYSGSTLQSGIPSRFSGSGSGTDFTLTISSLEPEDFAMYYCQQHNEYPWTFGGGTKLEIK

SEQ ID NO: 31 - protein, 118 aa, Artificial sequence
  Synthetic
EVQLQQSGPVLVKPGASVKMSCKASGYTFTDFYMNWAKQSHGKSLEWVGVINPYNGGINYNQKFKGKATLTVDKSSSTAYMEFNSLTSEDSAVYYCARRMEYHAMDYWGQGTSVTVSS

SEQ ID NO: 32 - protein, 111 aa, Artificial sequence
  Synthetic
DIVLTQSPASLAVSLGQRATISCRASESVDNYGFDFIHWYQQKPGQPPKLLIYRASNLESGIPARFSGSGSRPDFTLTINPVETDDVATYYCQQSIKDPWTFGGGTKLEIK

SEQ ID NO: 33 - protein, 121 aa, Artificial sequence
  Synthetic
EIQLQQSGPELVKPGTSVKVSCKASGYDFTRYNMYWVKQSHGKSLEWIGYIDPYNGDTRYNQKFKGKATLTVDKSSSTAYMHLNSLTSEDSAVYYCEAIYYDMEGYALDYWGQGTSVTVSS

SEQ ID NO: 34 - protein, 113 aa, Artificial sequence
  Synthetic
DIVMSQSPSSLAVSVGEKVTMSCKSSQSLLYSNNQKNYLAWYQQKPGQSPKKLIYWASTRESGVPDRLTGSGSGTDFTLTISSVTAEDLAVYYCQQFYSYPWTFGGGTKLEIK

SEQ ID NO: 35 - protein, 122 aa, Artificial sequence
  Synthetic
EVLLHQSGPELVKPGASVKISCKASGYTFTDFNMDWVRQRHGQSLEWIGDVNPNNGGTIYNQKFKGKATLTVDKSSSTVYMDLRSLTSDDTAVYYCVRLGTSDYGEAWFISWGQGTVVTVSA

SEQ ID NO: 36 - protein, 111 aa, Artificial sequence
  Synthetic
DIVLTQSPASLAVSLGQRATISCRASQSVSTPRYSFMHWYQQKPGQPPKLLIKYASNLESGFPARFTGSGSGTDFTLNIHPVEEEDTAAYYCQQSWAIPWTFGGGTKLEIK

SEQ ID NO: 37 - protein, 122 aa, Artificial sequence
  Synthetic
QVQLQQSGAEQVKPGASVKMSCKASGYTFTTYPIEWMKQNHGESLDWIGNIHPYNDDTEYNEKFKGKATLTVEKSSNTVFLELSRLTSDDSAVYYCVRGDYYNSRYWYFDVWGTGTTVTVSS

SEQ ID NO: 38 - protein, 107 aa, Artificial sequence
  Synthetic
DIVMTQSQKFMSTTVGDRVSITCKAGQNVRTAVAWYQQKPGQSPKLLIYSASTRYIGVPERFTGSGSGTDFTLTITNMQSEDLADYFCQQYSSFPLTFGTGTKLELK

SEQ ID NO: 39 - protein, 122 aa, Artificial sequence
  Synthetic
QVQLQQSGPELVKPGASVKISCKASGNAFSNSWMNWVKQRPGKGLEWIGRVYSEDGDTQYNGKFRDKATLTADRSTSTAYMQLSSLTSEDSAVYFCARWLIYYGTYGAMDYWGQGTSVTVSS

SEQ ID NO: 40 - protein, 107 aa, Artificial sequence
  Synthetic
DIQMTQTTSSLSASLGDRVTISCRASQDINNYLNWYHQKPDGTVKLLIYFTSRPYSGVPSRFSGSGSGTDYSLTISNLEQEDIATYFCQQGNTLPWTFGGGTKLEIK

SEQ ID NO: 41 - protein, 122 aa, Artificial sequence
  Synthetic
QVQLQQSGPELVKPGASVKISCKASGYEFSSIWMNWVKQRPGEGLEWIGRIFPGDGEINYNEKFKGKATLTADRSSTTAYMQLSSLTSEDSAVYFCAGWRIYYGTYGAMDYWGQGTSVTVSS

SEQ ID NO: 42 - protein, 107 aa, Artificial sequence
  Synthetic
DIQMTQITSSLSASLGDRVTISCRASQDIGNYLNWYQQKPDGTVKLLIYYTSRLHSGVPSRFSGSGSRTDYSLTISNLQQEDIATYFCQQGNTLPWTFGGGTKLEIK

SEQ ID NO: 43 - protein, 122 aa, Artificial sequence
  Synthetic
QVQLQQSGPEVVRPGASVKISCKASGYAFSSSWMNWMKQRPGKALEWIGRIYPGDGDTNYSGEFKVKATLTADKSSSTAYMQLSSLTSEDSAVYFCAREGSYYSNPWYFDYWGQGTTLTVSS

SEQ ID NO: 44 - protein, 112 aa, Artificial sequence
  Synthetic
DVLVTQTPLSLPVSLGDQASISCRSSQSIVQSNGDTYLEWYLQKPGQSPKLLIYKVSNRFSGVPDRFSGSGSGTDFTLKISRVEAEDLGVYYCFQGSHVPFTFGSGTKLEIK

SEQ ID NO: 45 - protein, 114 aa, Artificial sequence
  Synthetic
QIQLVQSGPELKKPGETVKISCKASGYTFTTYGMSWVKQTPGKGLTWMGWINTDSGVPTYADDFKRRFAFSLEASATTAYLQINNLKNEDTATYFCARGGPDYWGQGTTLTVSS

SEQ ID NO: 46 - protein, 107 aa, Artificial sequence
  Synthetic
DILLTQSPATLSVTPGETVSLSCRASQSIYKNLHWYHQKSHRSPRLLIKYASDSISGIPSRFTGSGSGTNFTLSINSVKPEDEGIYYCLQGYIVPLTFGAGTKLELK

SEQ ID NO: 47 - protein, 121 aa, Artificial sequence
  Synthetic
QNQLVQSGPELKKPGETVKISCKASGYTFTTAGMQWVQKMPGKGFKWIGWINTLSGEPKYAEDFKGRFAFSLETSATTAYLQISNLKNEDTATYFCARAYYSNLYWYFDVWGTGTTVTVSS

SEQ ID NO: 48 - protein, 107 aa, Artificial sequence
  Synthetic
DIVMTQSQKFMSTTVGDRVTITCKASQNVVTAVAWYQQKPGQSPKVLIYSASNRYSGVPDRFTGSGSGTDFTLTISNMHSEDLANYFCHQYSSYPFTFGSGTKLEIK

SEQ ID NO: 49 - protein, 115 aa, Artificial sequence
  Synthetic
AVQLQQSGVELVRPGASVKLSCTGSDFNIKEDYIHWVKQRPEQGLEWIGWIDPENGDTEYASKFQGKATITADTSSNTAYLQLSSLTSEDTAVYYCATYGSLDYWGQGTTLTVSS

SEQ ID NO: 50 - protein, 107 aa, Artificial sequence
  Synthetic
DIKMTQSPSSMYAFLGERVTITCKASQGINSFLTWFQQKPGKSPKTLIYRANRLVDGVPSRFSGSGSGQDYSLTISSLEYEDVGIYYCLQYDEFPRTFGGGTKLEIK

SEQ ID NO: 51 - protein, 126 aa, Artificial sequence
  Synthetic
EVQLQQSGAELVKPGASVKLSCTGSGFNIKDYYIHWVKQRTEQGLEWMGRIDPEDGETEYAPKFQDEATITSDTSSNTAYLQLSSLTYEDTAVYYCAREAAGYFYRGSSYGYFDVWGTGTTVTVSS

SEQ ID NO: 52 - protein, 107 aa, Artificial sequence
  Synthetic
DFQMTQTTSFLSASLGDRVTISCSASQDISNYLNWYQQKPDGTVKLLIYYTSSLHSGVPSRFSGSGSGTDYSLTISNLEPEDIATYYCQQYSMLPYTFGGGTKLEME

SEQ ID NO: 53 - protein, 113 aa, Artificial sequence
  Synthetic
QVQLKESGPGLVAPSQSLSITCTVSGLSLNNYGVSWVRQPPGKGLEWLGVIWGDGSTNYHSALISRLSISKDNSKSQVFLKLNSLHTDDTATYYCAINWGDYWGQGTTLIVSS

SEQ ID NO: 54 - protein, 107 aa, Artificial sequence
  Synthetic
DIKMTQSPSSMNASLGERVTITCKASQDINSYLSWFQQKPGKSPKILIYRANRLIDGVPSRFSGSGSGQDYSLTISSLEYEDMGIYYCLQYGVFPLTFGAGTKLELK

SEQ ID NO: 55 - protein, 123 aa, Artificial sequence
  Synthetic
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARAPPPNWDEYYFDYWGQGTTLTVSS

SEQ ID NO: 56 - protein, 106 aa, Artificial sequence
  Synthetic
DIQMTQSPASLSASVGETVTITCRASGNIHNSLAWYQQKQGKSPQLLVHNAKTLADGVPSRFSGSGSGTQYSLKINSLQPEDFGSYYCQHFWSTPPFGGGTKLEIK

SEQ ID NO: 57 - protein, 119 aa, Artificial sequence
  Synthetic
QVQLKESGPGLVAPSQSLSITCTVSGFSLSRYTVHWVRQPPGKGLEWLGMIWGGGSTDYNSALKSRLSISKDNSKSQVFLKMNSLQTDDTAMYYCARPHDFDAGGFAYWGQGTLVTVSA

SEQ ID NO: 58 - protein, 106 aa, Artificial sequence
  Synthetic
QIVLTQSPAILSTSPGEKVTMTCSASSRVNYMYWYQQKPGSSPRLLIYDTSNLASGVPVRFSGSGSGTSYSLTISRMEAEDAATYYCQQWTSYPLTFGAGTKLELK

SEQ ID NO: 59 - protein, 118 aa, Artificial sequence
  Synthetic
QVHLQQPGTELMKPGASVKLSCKATGYTFRDYWLEWVKQRPGHGLEWIGEILPGSGNAYYNEKFKGKATFTADTSSNTAYMQLSSLTTEDSAIYYCARVYSNWYFDAWGKGTTVTVSS

SEQ ID NO: 60 - protein, 109 aa, Artificial sequence
  Synthetic
QAVVTQESALTTSPGETVTLTCRSSTGAVTTSNYANWVQEKPDHLFTGLIGGTNNQAPGVPARFSGSLIGDKAALTISGAQTEDEAIYFCALWFSNHWVFGGGTKLTVL

SEQ ID NO: 61 - protein, 118 aa, Artificial sequence
  Synthetic
EVQLVESGGGLVKPGGSLKLSCAASGFTFSDYGMNWFRQAPEKGLEWVAYISRGSHTIYYADTVKGRFTISRDNAKNTLFLQMTSLRSNDTAMYYCVRMAGYYAMDYWGQGTSVTVSS

SEQ ID NO: 62 - protein, 115 aa, Artificial sequence
  Synthetic
QLVLTQSSSASFSLGASAKLTCTLSSQHSSYIIEWYQQQPLKPPKYVMELKKDGSHSTGDGIPDRFSGSSSGADRYLSISNIQPEDEAIYICGVGDTIKEQFVYVFGGGTKVTVL

SEQ ID NO: 63 - protein, 118 aa, Artificial sequence
  Synthetic
EVQLVESGGGFVKPGGSLKLSCAASGFTFSDYGMNWFRQAPEKGLEWVAYISRGSHTIYYADTVKGRFTISRDNAENTLFLQMTSLRSNDTAMYYCVRMAGYYAMDYWGQGTSLTVSS

SEQ ID NO: 64 - protein, 115 aa, Artificial sequence
  Synthetic
QLVLTQSSSASFSLGASAKLTCTLSSQHSSYIIEWYQQQPLKPPKYVMEVKKDGSHSTGDGIPDRFSGSSSGADRYLSISNIQSEDEAIYICGVGDTIKEQFVYVFGGGTKVTVL

SEQ ID NO: 65 - protein, 118 aa, Artificial sequence
  Synthetic
EVKLVESGGGLVLPGGSLKISCAASGFTFSDYYMYWIRQTPDKRLEWVAYISNTGYSTFYPDSVKGRFTVSRDNAKNTLYLQMSRLQSEDTAMYYCARHGILYAMDYWGQGTSVTVSS

SEQ ID NO: 66 - protein, 115 aa, Artificial sequence
  Synthetic
QPVLTQSSSASFSLGASAKLTCTLSSQYSTYTIGWYQQQPLKPPKYVMELKHDGSRHTGDGIPDRFSGSSSGADRYLSISNIQPEDEATYICGVGDTIKEQFMYVFGGGTKVTVL

SEQ ID NO: 67 - protein, 118 aa, Artificial sequence
  Synthetic
EVQLVESGGGLVKPGGSLKLSCAASGFTFSDYVMSWIRQTPEKRLEWVATISDAGSYTFYPDNLKGRFTISRDNAKNNLYLQMNHLTSEDTAFYYCAIIYFGNYGGYWGQGTLVTVSV

SEQ ID NO: 68 - protein, 109 aa, Artificial sequence
  Synthetic
QAVVTQESALTTSPGGTVVLTCRSNTGAVTTSNYANWVQEKPDHLFTGLIGGTSNRAPGVPVRFSGSLIGDKAALTITGAQTEDDAMYFCALWYSTHWVFGGGTKLTVL

SEQ ID NO: 69 - protein, 116 aa, Artificial sequence
  Synthetic
EVQLVESGGDLVKPGESLKLSCEASGFTFSNYAMSWVRQTPDRRLEWVATISSIGSFTYYSDSVKGRFTISRDNVKSTLHLQMNNLKSGDTAIYFCARLGVYFDSWGQGTTLTVSS

SEQ ID NO: 70 - protein, 115 aa, Artificial sequence
  Synthetic
QLVLTQSSSASFSLGASAKLTCTLSSQHSTYTIEWYQQQPHKPPKFVMEIKKDGSHYTGDGIPDRFSGSSSGADRYLSISNIQPEDETIYICGVSDMIKDQFVYVFGGGTKVTVL

SEQ ID NO: 71 - protein, 117 aa, Artificial sequence
  Synthetic
QVQLKQSGPGLVQPSQSLSITCTVSGFSLTGYGVQWVRQSPGKGLEWLGVIWGGGGTDYNAAFISRLNIIKDNSKSQVFFKMNSLQADDTAIYYCARYSKYGHFDVWGTGTAVTVSS

SEQ ID NO: 72 - protein, 110 aa, Artificial sequence
  Synthetic
QAVVTQESALTTSPGETVTLTCRSSTGAVTTSNYANWVQEKPDHLFTGLIGGTYNRVPGVPARFSGSLIGDKAALTITGAQTEDEAIYFCALWYSNHFWVFGGGTKLTVL

SEQ ID NO: 73 - protein, 123 aa, Artificial sequence
  Synthetic
QVTLKESGPGILQPSQTLSLTCSFSGFSLSTSGMGVGWIRQPSGKGLEWLAHIWWDDDEYYNPALKSRLTISKDTSKNQVFLKIANVDTADTATYYCARISIPYGYYDWFFDVWGTGTTVTVS

SEQ ID NO: 74 - protein, 109 aa, Artificial sequence
  Synthetic
QAVVTQESALTTSPGETVTLTCRSSTGAVTTRNYANWVQEKPDHLFTGLIGGTNNRAPGVPARFSGSLIGDKAALTITGAQTEDEAIYFCALWYSNHFIFGSGTKVTVL

SEQ ID NO: 75 - protein, 120 aa, Artificial sequence
  Synthetic
QVTLKESGPGILQPSQTLSLTCSFSGFSLSTFPMGVGWIRQPSGKGLEWLAHIWWDDDEYYNPVLKSRLTISKDTSKKQVYLRIANVDTVDTATYYCTRANWDRWYFDVWGTGTTVTVSS

SEQ ID NO: 76 - protein, 113 aa, Artificial sequence
  Synthetic
QLVLTQSSSASFSLGASAKLTCTLSRQHSAYTIEWYQQQPLKPPKYVMEVKKDGSHSTGDGIPDRFSGSSSGADRYLSISNIQPEDEAIYICGVGDTIKEHFVFGGGTKVTVL

SEQ ID NO: 77 - protein, 119 aa, Artificial sequence
  Synthetic
QVTLKESGPGILQPSQTLSLTCSFSGFSLSTFGMGVGWIRQPSGKGLEWLAHIWWDDDKHYNPALKSRLTISKDTSRNQVFLKIANVDTADGATYFCARDNWVSSLDYWGQGTSVIVSS

SEQ ID NO: 78 - protein, 115 aa, Artificial sequence
  Synthetic
QPVITQSSSASFSLGASAELTCTLSSQHSTYTIEWYQQQPLKPPKYVMGLKKDGSHSTGDGIPDRFSGSSSGADRYLSISNIQPEDEAIYICGVSDTIKEQFVYVFGGGTKVTVL

SEQ ID NO: 79 - protein, 120 aa, Artificial sequence
  Synthetic
QVTLKESGPGILQPSQTLSLTCSFSGFSLSTFPLGVGWIRQPSGKGLEWLAHVWWNDDDYYNPALKSRLTISKDTSNTRVFLSVASVDTAETATYYCARLNWDRWYFDVWGTGTTVTVSS

SEQ ID NO: 80 - protein, 113 aa, Artificial sequence
  Synthetic
QLVLTQSSSASFSLGASAKLTCTLSSQHSAYTVEWYQQQPLKPPKYVMELKKDGSHSTGDGIPDRFSGSSSGADRYLSISNIQPEDEAIYICGVGDTVKEQFVFGGGTKVTVL

SEQ ID NO: 81 - protein, 120 aa, Artificial sequence
  Synthetic
QVTLKESGPGILQPSQTLSLTCSFSGFSLSTFDRGVGWIRQPSGKGLEWLAHIWWNDKKFYNPVLKSRLTISKDTSKNQVFLTIANVDSADTATYSCARIVWDKYYFDYWGQGIPLTVSS

SEQ ID NO: 82 - protein, 113 aa, Artificial sequence
  Synthetic
QLVLTQSSSASFSLGASAKLTCTLSSQHSAYTIEWYQQQPLKPPKFVMDLKKDGSHSTGDGIPARFSGSSSGADRYLTISNIQPEDEAIYICGVGDTIKEQYVFGGGTKVTVL

SEQ ID NO: 83 - protein, 8 aa, Artificial sequence
  CDR
GYTFTNYW

SEQ ID NO: 84 - protein, 8 aa, Artificial sequence
  CDR
IHPSDSDT

SEQ ID NO: 85 - protein, 6 aa, Artificial sequence
  CDR
AMQFDY

SEQ ID NO: 86 - protein, 5 aa, Artificial sequence
  CDR
NYWMH

SEQ ID NO: 87 - protein, 17 aa, Artificial sequence
  CDR
RIHPSDSDTEYNLQFKD

SEQ ID NO: 88 - protein, 4 aa, Artificial sequence
  CDR
QFDY

SEQ ID NO: 89 - protein, 7 aa, Artificial sequence
  CDR
GYTFTNY

SEQ ID NO: 90 - protein, 6 aa, Artificial sequence
  CDR
HPSDSD

SEQ ID NO: 91 - protein, 4 aa, Artificial sequence
  CDR
QFDY

SEQ ID NO: 92 - protein, 6 aa, Artificial sequence
  CDR
QDINTF

SEQ ID NO: 93 - protein, 2 aa, Artificial sequence
  CDR
RA

SEQ ID NO: 94 - protein, 9 aa, Artificial sequence
  CDR
LQYDEFPRT

SEQ ID NO: 95 - protein, 11 aa, Artificial sequence
  CDR
KASQDINTFLS

SEQ ID NO: 96 - protein, 7 aa, Artificial sequence
  CDR
RANRLVD

SEQ ID NO: 97 - protein, 9 aa, Artificial sequence
  CDR
LQYDEFPRT

SEQ ID NO: 98 - protein, 11 aa, Artificial sequence
  CDR
KASQDINTFLS

SEQ ID NO: 99 - protein, 7 aa, Artificial sequence
  CDR
RANRLVD

SEQ ID NO: 100 - protein, 9 aa, Artificial sequence
  CDR
LQYDEFPRT

SEQ ID NO: 101 - protein, 8 aa, Artificial sequence
  CDR
GYTFTSFW

SEQ ID NO: 102 - protein, 8 aa, Artificial sequence
  CDR
IDPSNGDT

SEQ ID NO: 103 - protein, 8 aa, Artificial sequence
  CDR
ASSYAMDY

SEQ ID NO: 104 - protein, 5 aa, Artificial sequence
  CDR
SFWMH

SEQ ID NO: 105 - protein, 17 aa, Artificial sequence
  CDR
NIDPSNGDTNYNEKFKS

SEQ ID NO: 106 - protein, 6 aa, Artificial sequence
  CDR
SYAMDY

SEQ ID NO: 107 - protein, 7 aa, Artificial sequence
  CDR
GYTFTSF

SEQ ID NO: 108 - protein, 6 aa, Artificial sequence
  CDR
DPSNGD

SEQ ID NO: 109 - protein, 6 aa, Artificial sequence
  CDR
SYAMDY

SEQ ID NO: 110 - protein, 6 aa, Artificial sequence
  CDR
QDINRF

SEQ ID NO: 111 - protein, 2 aa, Artificial sequence
  CDR
RA

SEQ ID NO: 112 - protein, 8 aa, Artificial sequence
  CDR
LQYDEFRT

SEQ ID NO: 113 - protein, 11 aa, Artificial sequence
  CDR
KASQDINRFLS

SEQ ID NO: 114 - protein, 7 aa, Artificial sequence
  CDR
RANRLVD

SEQ ID NO: 115 - protein, 8 aa, Artificial sequence
  CDR
LQYDEFRT

SEQ ID NO: 116 - protein, 11 aa, Artificial sequence
  CDR
KASQDINRFLS

SEQ ID NO: 117 - protein, 7 aa, Artificial sequence
  CDR
RANRLVD

SEQ ID NO: 118 - protein, 8 aa, Artificial sequence
  CDR
LQYDEFRT

SEQ ID NO: 119 - protein, 8 aa, Artificial sequence
  CDR
GYTFTSYW

SEQ ID NO: 120 - protein, 8 aa, Artificial sequence
  CDR
INPSNGGT

SEQ ID NO: 121 - protein, 6 aa, Artificial sequence
  CDR
AGQLDY

SEQ ID NO: 122 - protein, 5 aa, Artificial sequence
  CDR
SYWMD

SEQ ID NO: 123 - protein, 17 aa, Artificial sequence
  CDR
NINPSNGGTNYNEKFKT

SEQ ID NO: 124 - protein, 4 aa, Artificial sequence
  CDR
QLDY

SEQ ID NO: 125 - protein, 7 aa, Artificial sequence
  CDR
GYTFTSY

SEQ ID NO: 126 - protein, 6 aa, Artificial sequence
  CDR
NPSNGG

SEQ ID NO: 127 - protein, 4 aa, Artificial sequence
  CDR
QLDY

SEQ ID NO: 128 - protein, 6 aa, Artificial sequence
  CDR
QDINSY

SEQ ID NO: 129 - protein, 2 aa, Artificial sequence
  CDR
RA

SEQ ID NO: 130 - protein, 9 aa, Artificial sequence
  CDR
LQYNEFPRT

SEQ ID NO: 131 - protein, 11 aa, Artificial sequence
  CDR
KASQDINSYLS

SEQ ID NO: 132 - protein, 7 aa, Artificial sequence
  CDR
RANRLVD

SEQ ID NO: 133 - protein, 9 aa, Artificial sequence
  CDR
LQYNEFPRT

SEQ ID NO: 134 - protein, 11 aa, Artificial sequence
  CDR
KASQDINSYLS

SEQ ID NO: 135 - protein, 7 aa, Artificial sequence
  CDR
RANRLVD

SEQ ID NO: 136 - protein, 9 aa, Artificial sequence
  CDR
LQYNEFPRT

SEQ ID NO: 137 - protein, 8 aa, Artificial sequence
  CDR
GYTFTIYW

SEQ ID NO: 138 - protein, 8 aa, Artificial sequence
  CDR
IDPHSGDT

SEQ ID NO: 139 - protein, 12 aa, Artificial sequence
  CDR
ARRDYSYYVLDY

SEQ ID NO: 140 - protein, 5 aa, Artificial sequence
  CDR
IYWMQ

SEQ ID NO: 141 - protein, 17 aa, Artificial sequence
  CDR
RIDPHSGDTHYNENFTT

SEQ ID NO: 142 - protein, 10 aa, Artificial sequence
  CDR
RDYSYYVLDY

SEQ ID NO: 143 - protein, 7 aa, Artificial sequence
  CDR
GYTFTIY

SEQ ID NO: 144 - protein, 6 aa, Artificial sequence
  CDR
DPHSGD

SEQ ID NO: 145 - protein, 10 aa, Artificial sequence
  CDR
RDYSYYVLDY

SEQ ID NO: 146 - protein, 11 aa, Artificial sequence
  CDR
QSLVHINGDTY

SEQ ID NO: 147 - protein, 2 aa, Artificial sequence
  CDR
TV

SEQ ID NO: 148 - protein, 9 aa, Artificial sequence
  CDR
SQSTHVPYT

SEQ ID NO: 149 - protein, 16 aa, Artificial sequence
  CDR
RSSQSLVHINGDTYLH

SEQ ID NO: 150 - protein, 7 aa, Artificial sequence
  CDR
TVFNRFS

SEQ ID NO: 151 - protein, 9 aa, Artificial sequence
  CDR
SQSTHVPYT

SEQ ID NO: 152 - protein, 16 aa, Artificial sequence
  CDR
RSSQSLVHINGDTYLH

SEQ ID NO: 153 - protein, 7 aa, Artificial sequence
  CDR
TVFNRFS

SEQ ID NO: 154 - protein, 9 aa, Artificial sequence
  CDR
SQSTHVPYT

SEQ ID NO: 155 - protein, 8 aa, Artificial sequence
  CDR
GYTFTNFW

SEQ ID NO: 156 - protein, 8 aa, Artificial sequence
  CDR
IYPGSGDT

SEQ ID NO: 157 - protein, 6 aa, Artificial sequence
  CDR
TNQMDY

SEQ ID NO: 158 - protein, 5 aa, Artificial sequence
  CDR
NFWMH

SEQ ID NO: 159 - protein, 17 aa, Artificial sequence
  CDR
NIYPGSGDTYYNEKFRS

SEQ ID NO: 160 - protein, 4 aa, Artificial sequence
  CDR
QMDY

SEQ ID NO: 161 - protein, 7 aa, Artificial sequence
  CDR
GYTFTNF

SEQ ID NO: 162 - protein, 6 aa, Artificial sequence
  CDR
YPGSGD

SEQ ID NO: 163 - protein, 4 aa, Artificial sequence
  CDR
QMDY

SEQ ID NO: 164 - protein, 6 aa, Artificial sequence
  CDR
QDTNRY

SEQ ID NO: 165 - protein, 2 aa, Artificial sequence
  CDR
RT

SEQ ID NO: 166 - protein, 9 aa, Artificial sequence
  CDR
LQYAEFPYT

SEQ ID NO: 167 - protein, 11 aa, Artificial sequence
  CDR
TASQDTNRYLK

SEQ ID NO: 168 - protein, 7 aa, Artificial sequence
  CDR
RTNRLID

SEQ ID NO: 169 - protein, 9 aa, Artificial sequence
  CDR
LQYAEFPYT

SEQ ID NO: 170 - protein, 11 aa, Artificial sequence
  CDR
TASQDTNRYLK

SEQ ID NO: 171 - protein, 7 aa, Artificial sequence
  CDR
RTNRLID

SEQ ID NO: 172 - protein, 9 aa, Artificial sequence
  CDR
LQYAEFPYT

SEQ ID NO: 173 - protein, 8 aa, Artificial sequence
  CDR
GYTFTTYT

SEQ ID NO: 174 - protein, 8 aa, Artificial sequence
  CDR
INPSSGYT

SEQ ID NO: 175 - protein, 6 aa, Artificial sequence
  CDR
TLQLDY

SEQ ID NO: 176 - protein, 5 aa, Artificial sequence
  CDR
TYTMH

SEQ ID NO: 177 - protein, 17 aa, Artificial sequence
  CDR
YINPSSGYTQYNQKFKD

SEQ ID NO: 178 - protein, 4 aa, Artificial sequence
  CDR
QLDY

SEQ ID NO: 179 - protein, 7 aa, Artificial sequence
  CDR
GYTFTTY

SEQ ID NO: 180 - protein, 6 aa, Artificial sequence
  CDR
NPSSGY

SEQ ID NO: 181 - protein, 4 aa, Artificial sequence
  CDR
QLDY

SEQ ID NO: 182 - protein, 6 aa, Artificial sequence
  CDR
QDINSY

SEQ ID NO: 183 - protein, 2 aa, Artificial sequence
  CDR
RA

SEQ ID NO: 184 - protein, 8 aa, Artificial sequence
  CDR
LQYYELYT

SEQ ID NO: 185 - protein, 11 aa, Artificial sequence
  CDR
KASQDINSYLS

SEQ ID NO: 186 - protein, 7 aa, Artificial sequence
  CDR
RANRLVD

SEQ ID NO: 187 - protein, 8 aa, Artificial sequence
  CDR
LQYYELYT

SEQ ID NO: 188 - protein, 11 aa, Artificial sequence
  CDR
KASQDINSYLS

SEQ ID NO: 189 - protein, 7 aa, Artificial sequence
  CDR
RANRLVD

SEQ ID NO: 190 - protein, 8 aa, Artificial sequence
  CDR
LQYYELYT

SEQ ID NO: 191 - protein, 8 aa, Artificial sequence
  CDR
GYTFTSYW

SEQ ID NO: 192 - protein, 8 aa, Artificial sequence
  CDR
IYPGTGSS

SEQ ID NO: 193 - protein, 10 aa, Artificial sequence
  CDR
ARLKFEGIGY

SEQ ID NO: 194 - protein, 5 aa, Artificial sequence
  CDR
SYWIS

SEQ ID NO: 195 - protein, 17 aa, Artificial sequence
  CDR
DIYPGTGSSNYNEKFKS

SEQ ID NO: 196 - protein, 8 aa, Artificial sequence
  CDR
LKFEGIGY

SEQ ID NO: 197 - protein, 7 aa, Artificial sequence
  CDR
GYTFTSY

SEQ ID NO: 198 - protein, 6 aa, Artificial sequence
  CDR
YPGTGS

SEQ ID NO: 199 - protein, 8 aa, Artificial sequence
  CDR
LKFEGIGY

SEQ ID NO: 200 - protein, 5 aa, Artificial sequence
  CDR
SSVSY

SEQ ID NO: 201 - protein, 2 aa, Artificial sequence
  CDR
SI

SEQ ID NO: 202 - protein, 9 aa, Artificial sequence
  CDR
HQWSSYPWT

SEQ ID NO: 203 - protein, 10 aa, Artificial sequence
  CDR
SASSSVSYMH

SEQ ID NO: 204 - protein, 7 aa, Artificial sequence
  CDR
SISNLAS

SEQ ID NO: 205 - protein, 9 aa, Artificial sequence
  CDR
HQWSSYPWT

SEQ ID NO: 206 - protein, 10 aa, Artificial sequence
  CDR
SASSSVSYMH

SEQ ID NO: 207 - protein, 7 aa, Artificial sequence
  CDR
SISNLAS

SEQ ID NO: 208 - protein, 9 aa, Artificial sequence
  CDR
HQWSSYPWT

SEQ ID NO: 209 - protein, 8 aa, Artificial sequence
  CDR
GYTFTSYG

SEQ ID NO: 210 - protein, 8 aa, Artificial sequence
  CDR
IYPKLGTT

SEQ ID NO: 211 - protein, 13 aa, Artificial sequence
  CDR
ACPHYYATRGGDY

SEQ ID NO: 212 - protein, 5 aa, Artificial sequence
  CDR
SYGIS

SEQ ID NO: 213 - protein, 17 aa, Artificial sequence
  CDR
EIYPKLGTTYYNEKFKD

SEQ ID NO: 214 - protein, 11 aa, Artificial sequence
  CDR
PHYYATRGGDY

SEQ ID NO: 215 - protein, 7 aa, Artificial sequence
  CDR
GYTFTSY

SEQ ID NO: 216 - protein, 6 aa, Artificial sequence
  CDR
YPKLGT

SEQ ID NO: 217 - protein, 11 aa, Artificial sequence
  CDR
PHYYATRGGDY

SEQ ID NO: 218 - protein, 6 aa, Artificial sequence
  CDR
QSISNH

SEQ ID NO: 219 - protein, 2 aa, Artificial sequence
  CDR
YA

SEQ ID NO: 220 - protein, 9 aa, Artificial sequence
  CDR
QQSHSWPYT

SEQ ID NO: 221 - protein, 11 aa, Artificial sequence
  CDR
RASQSISNHLH

SEQ ID NO: 222 - protein, 7 aa, Artificial sequence
  CDR
YASQSIS

SEQ ID NO: 223 - protein, 9 aa, Artificial sequence
  CDR
QQSHSWPYT

SEQ ID NO: 224 - protein, 11 aa, Artificial sequence
  CDR
RASQSISNHLH

SEQ ID NO: 225 - protein, 7 aa, Artificial sequence
  CDR
YASQSIS

SEQ ID NO: 226 - protein, 9 aa, Artificial sequence
  CDR
QQSHSWPYT

SEQ ID NO: 227 - protein, 8 aa, Artificial sequence
  CDR
GYTFSDYF

SEQ ID NO: 228 - protein, 8 aa, Artificial sequence
  CDR
INSYSGGT

SEQ ID NO: 229 - protein, 6 aa, Artificial sequence
  CDR
ARWMDY

SEQ ID NO: 230 - protein, 5 aa, Artificial sequence
  CDR
DYFMN

SEQ ID NO: 231 - protein, 17 aa, Artificial sequence
  CDR
IINSYSGGTSYNQKFKG

SEQ ID NO: 232 - protein, 4 aa, Artificial sequence
  CDR
WMDY

SEQ ID NO: 233 - protein, 7 aa, Artificial sequence
  CDR
GYTFSDY

SEQ ID NO: 234 - protein, 6 aa, Artificial sequence
  CDR
NSYSGG

SEQ ID NO: 235 - protein, 4 aa, Artificial sequence
  CDR
WMDY

SEQ ID NO: 236 - protein, 6 aa, Artificial sequence
  CDR
ESISKH

SEQ ID NO: 237 - protein, 2 aa, Artificial sequence
  CDR
SG

SEQ ID NO: 238 - protein, 9 aa, Artificial sequence
  CDR
QQHNEYPWT

SEQ ID NO: 239 - protein, 11 aa, Artificial sequence
  CDR
RTSESISKHLA

SEQ ID NO: 240 - protein, 7 aa, Artificial sequence
  CDR
SGSTLQS

SEQ ID NO: 241 - protein, 9 aa, Artificial sequence
  CDR
QQHNEYPWT

SEQ ID NO: 242 - protein, 11 aa, Artificial sequence
  CDR
RTSESISKHLA

SEQ ID NO: 243 - protein, 7 aa, Artificial sequence
  CDR
SGSTLQS

SEQ ID NO: 244 - protein, 9 aa, Artificial sequence
  CDR
QQHNEYPWT

SEQ ID NO: 245 - protein, 8 aa, Artificial sequence
  CDR
GYTFTDFY

SEQ ID NO: 246 - protein, 8 aa, Artificial sequence
  CDR
INPYNGGI

SEQ ID NO: 247 - protein, 11 aa, Artificial sequence
  CDR
ARRMEYHAMDY

SEQ ID NO: 248 - protein, 5 aa, Artificial sequence
  CDR
DFYMN

SEQ ID NO: 249 - protein, 17 aa, Artificial sequence
  CDR
VINPYNGGINYNQKFKG

SEQ ID NO: 250 - protein, 9 aa, Artificial sequence
  CDR
RMEYHAMDY

SEQ ID NO: 251 - protein, 7 aa, Artificial sequence
  CDR
GYTFTDF

SEQ ID NO: 252 - protein, 6 aa, Artificial sequence
  CDR
NPYNGG

SEQ ID NO: 253 - protein, 9 aa, Artificial sequence
  CDR
RMEYHAMDY

SEQ ID NO: 254 - protein, 10 aa, Artificial sequence
  CDR
ESVDNYGFDF

SEQ ID NO: 255 - protein, 2 aa, Artificial sequence
  CDR
RA

SEQ ID NO: 256 - protein, 9 aa, Artificial sequence
  CDR
QQSIKDPWT

SEQ ID NO: 257 - protein, 15 aa, Artificial sequence
  CDR
RASESVDNYGFDFIH

SEQ ID NO: 258 - protein, 7 aa, Artificial sequence
  CDR
RASNLES

SEQ ID NO: 259 - protein, 9 aa, Artificial sequence
  CDR
QQSIKDPWT

SEQ ID NO: 260 - protein, 15 aa, Artificial sequence
  CDR
RASESVDNYGFDFIH

SEQ ID NO: 261 - protein, 7 aa, Artificial sequence
  CDR
RASNLES

SEQ ID NO: 262 - protein, 9 aa, Artificial sequence
  CDR
QQSIKDPWT

SEQ ID NO: 263 - protein, 8 aa, Artificial sequence
  CDR
GYDFTRYN

SEQ ID NO: 264 - protein, 8 aa, Artificial sequence
  CDR
IDPYNGDT

SEQ ID NO: 265 - protein, 14 aa, Artificial sequence
  CDR
EAIYYDMEGYALDY

SEQ ID NO: 266 - protein, 5 aa, Artificial sequence
  CDR
RYNMY

SEQ ID NO: 267 - protein, 17 aa, Artificial sequence
  CDR
YIDPYNGDTRYNQKFKG

SEQ ID NO: 268 - protein, 12 aa, Artificial sequence
  CDR
IYYDMEGYALDY

SEQ ID NO: 269 - protein, 7 aa, Artificial sequence
  CDR
GYDFTRY

SEQ ID NO: 270 - protein, 6 aa, Artificial sequence
  CDR
DPYNGD

SEQ ID NO: 271 - protein, 12 aa, Artificial sequence
  CDR
IYYDMEGYALDY

SEQ ID NO: 272 - protein, 12 aa, Artificial sequence
  CDR
QSLLYSNNQKNY

SEQ ID NO: 273 - protein, 2 aa, Artificial sequence
  CDR
WA

SEQ ID NO: 274 - protein, 9 aa, Artificial sequence
  CDR
QQFYSYPWT

SEQ ID NO: 275 - protein, 17 aa, Artificial sequence
  CDR
KSSQSLLYSNNQKNYLA

SEQ ID NO: 276 - protein, 7 aa, Artificial sequence
  CDR
WASTRES

SEQ ID NO: 277 - protein, 9 aa, Artificial sequence
  CDR
QQFYSYPWT

SEQ ID NO: 278 - protein, 17 aa, Artificial sequence
  CDR
KSSQSLLYSNNQKNYLA

SEQ ID NO: 279 - protein, 7 aa, Artificial sequence
  CDR
WASTRES

SEQ ID NO: 280 - protein, 9 aa, Artificial sequence
  CDR
QQFYSYPWT

SEQ ID NO: 281 - protein, 8 aa, Artificial sequence
  CDR
GYTFTDFN

SEQ ID NO: 282 - protein, 8 aa, Artificial sequence
  CDR
VNPNNGGT

SEQ ID NO: 283 - protein, 15 aa, Artificial sequence
  CDR
VRLGTSDYGEAWFIS

SEQ ID NO: 284 - protein, 5 aa, Artificial sequence
  CDR
DFNMD

SEQ ID NO: 285 - protein, 17 aa, Artificial sequence
  CDR
DVNPNNGGTIYNQKFKG

SEQ ID NO: 286 - protein, 13 aa, Artificial sequence
  CDR
LGTSDYGEAWFIS

SEQ ID NO: 287 - protein, 7 aa, Artificial sequence
  CDR
GYTFTDF

SEQ ID NO: 288 - protein, 6 aa, Artificial sequence
  CDR
NPNNGG

SEQ ID NO: 289 - protein, 13 aa, Artificial sequence
  CDR
LGTSDYGEAWFIS

SEQ ID NO: 290 - protein, 10 aa, Artificial sequence
  CDR
QSVSTPRYSF

SEQ ID NO: 291 - protein, 2 aa, Artificial sequence
  CDR
YA

SEQ ID NO: 292 - protein, 9 aa, Artificial sequence
  CDR
QQSWAIPWT

SEQ ID NO: 293 - protein, 15 aa, Artificial sequence
  CDR
RASQSVSTPRYSFMH

SEQ ID NO: 294 - protein, 7 aa, Artificial sequence
  CDR
YASNLES

SEQ ID NO: 295 - protein, 9 aa, Artificial sequence
  CDR
QQSWAIPWT

SEQ ID NO: 296 - protein, 15 aa, Artificial sequence
  CDR
RASQSVSTPRYSFMH

SEQ ID NO: 297 - protein, 7 aa, Artificial sequence
  CDR
YASNLES

SEQ ID NO: 298 - protein, 9 aa, Artificial sequence
  CDR
QQSWAIPWT

SEQ ID NO: 299 - protein, 8 aa, Artificial sequence
  CDR
GYTFTTYP

SEQ ID NO: 300 - protein, 8 aa, Artificial sequence
  CDR
IHPYNDDT

SEQ ID NO: 301 - protein, 15 aa, Artificial sequence
  CDR
VRGDYYNSRYWYFDV

SEQ ID NO: 302 - protein, 5 aa, Artificial sequence
  CDR
TYPIE

SEQ ID NO: 303 - protein, 17 aa, Artificial sequence
  CDR
NIHPYNDDTEYNEKFKG

SEQ ID NO: 304 - protein, 13 aa, Artificial sequence
  CDR
GDYYNSRYWYFDV

SEQ ID NO: 305 - protein, 7 aa, Artificial sequence
  CDR
GYTFTTY

SEQ ID NO: 306 - protein, 6 aa, Artificial sequence
  CDR
HPYNDD

SEQ ID NO: 307 - protein, 13 aa, Artificial sequence
  CDR
GDYYNSRYWYFDV

SEQ ID NO: 308 - protein, 6 aa, Artificial sequence
  CDR
QNVRTA

SEQ ID NO: 309 - protein, 2 aa, Artificial sequence
  CDR
SA

SEQ ID NO: 310 - protein, 9 aa, Artificial sequence
  CDR
QQYSSFPLT

SEQ ID NO: 311 - protein, 11 aa, Artificial sequence
  CDR
KAGQNVRTAVA

SEQ ID NO: 312 - protein, 7 aa, Artificial sequence
  CDR
SASTRYI

SEQ ID NO: 313 - protein, 9 aa, Artificial sequence
  CDR
QQYSSFPLT

SEQ ID NO: 314 - protein, 11 aa, Artificial sequence
  CDR
KAGQNVRTAVA

SEQ ID NO: 315 - protein, 7 aa, Artificial sequence
  CDR
SASTRYI

SEQ ID NO: 316 - protein, 9 aa, Artificial sequence
  CDR
QQYSSFPLT

SEQ ID NO: 317 - protein, 8 aa, Artificial sequence
  CDR
GNAFSNSW

SEQ ID NO: 318 - protein, 8 aa, Artificial sequence
  CDR
VYSEDGDT

SEQ ID NO: 319 - protein, 15 aa, Artificial sequence
  CDR
ARWLIYYGTYGAMDY

SEQ ID NO: 320 - protein, 5 aa, Artificial sequence
  CDR
NSWMN

SEQ ID NO: 321 - protein, 17 aa, Artificial sequence
  CDR
RVYSEDGDTQYNGKFRD

SEQ ID NO: 322 - protein, 13 aa, Artificial sequence
  CDR
WLIYYGTYGAMDY

SEQ ID NO: 323 - protein, 7 aa, Artificial sequence
  CDR
GNAFSNS

SEQ ID NO: 324 - protein, 6 aa, Artificial sequence
  CDR
YSEDGD

SEQ ID NO: 325 - protein, 13 aa, Artificial sequence
  CDR
WLIYYGTYGAMDY

SEQ ID NO: 326 - protein, 6 aa, Artificial sequence
  CDR
QDINNY

SEQ ID NO: 327 - protein, 2 aa, Artificial sequence
  CDR
FT

SEQ ID NO: 328 - protein, 9 aa, Artificial sequence
  CDR
QQGNTLPWT

SEQ ID NO: 329 - protein, 11 aa, Artificial sequence
  CDR
RASQDINNYLN

SEQ ID NO: 330 - protein, 7 aa, Artificial sequence
  CDR
FTSRPYS

SEQ ID NO: 331 - protein, 9 aa, Artificial sequence
  CDR
QQGNTLPWT

SEQ ID NO: 332 - protein, 11 aa, Artificial sequence
  CDR
RASQDINNYLN

SEQ ID NO: 333 - protein, 7 aa, Artificial sequence
  CDR
FTSRPYS

SEQ ID NO: 334 - protein, 9 aa, Artificial sequence
  CDR
QQGNTLPWT

SEQ ID NO: 335 - protein, 8 aa, Artificial sequence
  CDR
GYEFSSIW

SEQ ID NO: 336 - protein, 8 aa, Artificial sequence
  CDR
IFPGDGEI

SEQ ID NO: 337 - protein, 15 aa, Artificial sequence
  CDR
AGWRIYYGTYGAMDY

SEQ ID NO: 338 - protein, 5 aa, Artificial sequence
  CDR
SIWMN

SEQ ID NO: 339 - protein, 17 aa, Artificial sequence
  CDR
RIFPGDGEINYNEKFKG

SEQ ID NO: 340 - protein, 13 aa, Artificial sequence
  CDR
WRIYYGTYGAMDY

SEQ ID NO: 341 - protein, 7 aa, Artificial sequence
  CDR
GYEFSSI

SEQ ID NO: 342 - protein, 6 aa, Artificial sequence
  CDR
FPGDGE

SEQ ID NO: 343 - protein, 13 aa, Artificial sequence
  CDR
WRIYYGTYGAMDY

SEQ ID NO: 344 - protein, 6 aa, Artificial sequence
  CDR
QDIGNY

SEQ ID NO: 345 - protein, 2 aa, Artificial sequence
  CDR
YT

SEQ ID NO: 346 - protein, 9 aa, Artificial sequence
  CDR
QQGNTLPWT

SEQ ID NO: 347 - protein, 11 aa, Artificial sequence
  CDR
RASQDIGNYLN

SEQ ID NO: 348 - protein, 7 aa, Artificial sequence
  CDR
YTSRLHS

SEQ ID NO: 349 - protein, 9 aa, Artificial sequence
  CDR
QQGNTLPWT

SEQ ID NO: 350 - protein, 11 aa, Artificial sequence
  CDR
RASQDIGNYLN

SEQ ID NO: 351 - protein, 7 aa, Artificial sequence
  CDR
YTSRLHS

SEQ ID NO: 352 - protein, 9 aa, Artificial sequence
  CDR
QQGNTLPWT

SEQ ID NO: 353 - protein, 8 aa, Artificial sequence
  CDR
GYAFSSSW

SEQ ID NO: 354 - protein, 8 aa, Artificial sequence
  CDR
IYPGDGDT

SEQ ID NO: 355 - protein, 15 aa, Artificial sequence
  CDR
AREGSYYSNPWYFDY

SEQ ID NO: 356 - protein, 5 aa, Artificial sequence
  CDR
SSWMN

SEQ ID NO: 357 - protein, 17 aa, Artificial sequence
  CDR
RIYPGDGDTNYSGEFKV

SEQ ID NO: 358 - protein, 13 aa, Artificial sequence
  CDR
EGSYYSNPWYFDY

SEQ ID NO: 359 - protein, 7 aa, Artificial sequence
  CDR
GYAFSSS

SEQ ID NO: 360 - protein, 6 aa, Artificial sequence
  CDR
YPGDGD

SEQ ID NO: 361 - protein, 13 aa, Artificial sequence
  CDR
EGSYYSNPWYFDY

SEQ ID NO: 362 - protein, 11 aa, Artificial sequence
  CDR
QSIVQSNGDTY

SEQ ID NO: 363 - protein, 2 aa, Artificial sequence
  CDR
KV

SEQ ID NO: 364 - protein, 9 aa, Artificial sequence
  CDR
FQGSHVPFT

SEQ ID NO: 365 - protein, 16 aa, Artificial sequence
  CDR
RSSQSIVQSNGDTYLE

SEQ ID NO: 366 - protein, 7 aa, Artificial sequence
  CDR
KVSNRFS

SEQ ID NO: 367 - protein, 9 aa, Artificial sequence
  CDR
FQGSHVPFT

SEQ ID NO: 368 - protein, 16 aa, Artificial sequence
  CDR
RSSQSIVQSNGDTYLE

SEQ ID NO: 369 - protein, 7 aa, Artificial sequence
  CDR
KVSNRFS

SEQ ID NO: 370 - protein, 9 aa, Artificial sequence
  CDR
FQGSHVPFT

SEQ ID NO: 371 - protein, 8 aa, Artificial sequence
  CDR
GYTFTTYG

SEQ ID NO: 372 - protein, 8 aa, Artificial sequence
  CDR
INTDSGVP

SEQ ID NO: 373 - protein, 7 aa, Artificial sequence
  CDR
ARGGPDY

SEQ ID NO: 374 - protein, 5 aa, Artificial sequence
  CDR
TYGMS

SEQ ID NO: 375 - protein, 17 aa, Artificial sequence
  CDR
WINTDSGVPTYADDFKR

SEQ ID NO: 376 - protein, 5 aa, Artificial sequence
  CDR
GGPDY

SEQ ID NO: 377 - protein, 7 aa, Artificial sequence
  CDR
GYTFTTY

SEQ ID NO: 378 - protein, 6 aa, Artificial sequence
  CDR
NTDSGV

SEQ ID NO: 379 - protein, 5 aa, Artificial sequence
  CDR
GGPDY

SEQ ID NO: 380 - protein, 6 aa, Artificial sequence
  CDR
QSIYKN

SEQ ID NO: 381 - protein, 2 aa, Artificial sequence
  CDR
YA

SEQ ID NO: 382 - protein, 9 aa, Artificial sequence
  CDR
LQGYIVPLT

SEQ ID NO: 383 - protein, 11 aa, Artificial sequence
  CDR
RASQSIYKNLH

SEQ ID NO: 384 - protein, 7 aa, Artificial sequence
  CDR
YASDSIS

SEQ ID NO: 385 - protein, 9 aa, Artificial sequence
  CDR
LQGYIVPLT

SEQ ID NO: 386 - protein, 11 aa, Artificial sequence
  CDR
RASQSIYKNLH

SEQ ID NO: 387 - protein, 7 aa, Artificial sequence
  CDR
YASDSIS

SEQ ID NO: 388 - protein, 9 aa, Artificial sequence
  CDR
LQGYIVPLT

SEQ ID NO: 389 - protein, 8 aa, Artificial sequence
  CDR
GYTFTTAG

SEQ ID NO: 390 - protein, 8 aa, Artificial sequence
  CDR
INTLSGEP

SEQ ID NO: 391 - protein, 14 aa, Artificial sequence
  CDR
ARAYYSNLYWYFDV

SEQ ID NO: 392 - protein, 5 aa, Artificial sequence
  CDR
TAGMQ

SEQ ID NO: 393 - protein, 17 aa, Artificial sequence
  CDR
WINTLSGEPKYAEDFKG

SEQ ID NO: 394 - protein, 12 aa, Artificial sequence
  CDR
AYYSNLYWYFDV

SEQ ID NO: 395 - protein, 7 aa, Artificial sequence
  CDR
GYTFTTA

SEQ ID NO: 396 - protein, 6 aa, Artificial sequence
  CDR
NTLSGE

SEQ ID NO: 397 - protein, 12 aa, Artificial sequence
  CDR
AYYSNLYWYFDV

SEQ ID NO: 398 - protein, 6 aa, Artificial sequence
  CDR
QNVVTA

SEQ ID NO: 399 - protein, 2 aa, Artificial sequence
  CDR
SA

SEQ ID NO: 400 - protein, 9 aa, Artificial sequence
  CDR
HQYSSYPFT

SEQ ID NO: 401 - protein, 11 aa, Artificial sequence
  CDR
KASQNVVTAVA

SEQ ID NO: 402 - protein, 7 aa, Artificial sequence
  CDR
SASNRYS

SEQ ID NO: 403 - protein, 9 aa, Artificial sequence
  CDR
HQYSSYPFT

SEQ ID NO: 404 - protein, 11 aa, Artificial sequence
  CDR
KASQNVVTAVA

SEQ ID NO: 405 - protein, 7 aa, Artificial sequence
  CDR
SASNRYS

SEQ ID NO: 406 - protein, 9 aa, Artificial sequence
  CDR
HQYSSYPFT

SEQ ID NO: 407 - protein, 8 aa, Artificial sequence
  CDR
DFNIKEDY

SEQ ID NO: 408 - protein, 8 aa, Artificial sequence
  CDR
IDPENGDT

SEQ ID NO: 409 - protein, 8 aa, Artificial sequence
  CDR
ATYGSLDY

SEQ ID NO: 410 - protein, 5 aa, Artificial sequence
  CDR
EDYIH

SEQ ID NO: 411 - protein, 17 aa, Artificial sequence
  CDR
WIDPENGDTEYASKFQG

SEQ ID NO: 412 - protein, 6 aa, Artificial sequence
  CDR
YGSLDY

SEQ ID NO: 413 - protein, 7 aa, Artificial sequence
  CDR
DFNIKED

SEQ ID NO: 414 - protein, 6 aa, Artificial sequence
  CDR
DPENGD

SEQ ID NO: 415 - protein, 6 aa, Artificial sequence
  CDR
YGSLDY

SEQ ID NO: 416 - protein, 6 aa, Artificial sequence
  CDR
QGINSF

SEQ ID NO: 417 - protein, 2 aa, Artificial sequence
  CDR
RA

SEQ ID NO: 418 - protein, 9 aa, Artificial sequence
  CDR
LQYDEFPRT

SEQ ID NO: 419 - protein, 11 aa, Artificial sequence
  CDR
KASQGINSFLT

SEQ ID NO: 420 - protein, 7 aa, Artificial sequence
  CDR
RANRLVD

SEQ ID NO: 421 - protein, 9 aa, Artificial sequence
  CDR
LQYDEFPRT

SEQ ID NO: 422 - protein, 11 aa, Artificial sequence
  CDR
KASQGINSFLT

SEQ ID NO: 423 - protein, 7 aa, Artificial sequence
  CDR
RANRLVD

SEQ ID NO: 424 - protein, 9 aa, Artificial sequence
  CDR
LQYDEFPRT

SEQ ID NO: 425 - protein, 8 aa, Artificial sequence
  CDR
GFNIKDYY

SEQ ID NO: 426 - protein, 8 aa, Artificial sequence
  CDR
IDPEDGET

SEQ ID NO: 427 - protein, 19 aa, Artificial sequence
  CDR
AREAAGYFYRGSSYGYFDV

SEQ ID NO: 428 - protein, 5 aa, Artificial sequence
  CDR
DYYIH

SEQ ID NO: 429 - protein, 17 aa, Artificial sequence
  CDR
RIDPEDGETEYAPKFQD

SEQ ID NO: 430 - protein, 17 aa, Artificial sequence
  CDR
EAAGYFYRGSSYGYFDV

SEQ ID NO: 431 - protein, 7 aa, Artificial sequence
  CDR
GFNIKDY

SEQ ID NO: 432 - protein, 6 aa, Artificial sequence
  CDR
DPEDGE

SEQ ID NO: 433 - protein, 17 aa, Artificial sequence
  CDR
EAAGYFYRGSSYGYFDV

SEQ ID NO: 434 - protein, 6 aa, Artificial sequence
  CDR
QDISNY

SEQ ID NO: 435 - protein, 2 aa, Artificial sequence
  CDR
YT

SEQ ID NO: 436 - protein, 9 aa, Artificial sequence
  CDR
QQYSMLPYT

SEQ ID NO: 437 - protein, 11 aa, Artificial sequence
  CDR
SASQDISNYLN

SEQ ID NO: 438 - protein, 7 aa, Artificial sequence
  CDR
YTSSLHS

SEQ ID NO: 439 - protein, 9 aa, Artificial sequence
  CDR
QQYSMLPYT

SEQ ID NO: 440 - protein, 11 aa, Artificial sequence
  CDR
SASQDISNYLN

SEQ ID NO: 441 - protein, 7 aa, Artificial sequence
  CDR
YTSSLHS

SEQ ID NO: 442 - protein, 9 aa, Artificial sequence
  CDR
QQYSMLPYT

SEQ ID NO: 443 - protein, 8 aa, Artificial sequence
  CDR
GLSLNNYG

SEQ ID NO: 444 - protein, 7 aa, Artificial sequence
  CDR
IWGDGST

SEQ ID NO: 445 - protein, 7 aa, Artificial sequence
  CDR
AINWGDY

SEQ ID NO: 446 - protein, 5 aa, Artificial sequence
  CDR
NYGVS

SEQ ID NO: 447 - protein, 16 aa, Artificial sequence
  CDR
VIWGDGSTNYHSALIS

SEQ ID NO: 448 - protein, 5 aa, Artificial sequence
  CDR
NWGDY

SEQ ID NO: 449 - protein, 7 aa, Artificial sequence
  CDR
GLSLNNY

SEQ ID NO: 450 - protein, 5 aa, Artificial sequence
  CDR
WGDGS

SEQ ID NO: 451 - protein, 5 aa, Artificial sequence
  CDR
NWGDY

SEQ ID NO: 452 - protein, 6 aa, Artificial sequence
  CDR
QDINSY

SEQ ID NO: 453 - protein, 2 aa, Artificial sequence
  CDR
RA

SEQ ID NO: 454 - protein, 9 aa, Artificial sequence
  CDR
LQYGVFPLT

SEQ ID NO: 455 - protein, 11 aa, Artificial sequence
  CDR
KASQDINSYLS

SEQ ID NO: 456 - protein, 7 aa, Artificial sequence
  CDR
RANRLID

SEQ ID NO: 457 - protein, 9 aa, Artificial sequence
  CDR
LQYGVFPLT

SEQ ID NO: 458 - protein, 11 aa, Artificial sequence
  CDR
KASQDINSYLS

SEQ ID NO: 459 - protein, 7 aa, Artificial sequence
  CDR
RANRLID

SEQ ID NO: 460 - protein, 9 aa, Artificial sequence
  CDR
LQYGVFPLT

SEQ ID NO: 461 - protein, 10 aa, Artificial sequence
  CDR
GFSLSTSGMG

SEQ ID NO: 462 - protein, 7 aa, Artificial sequence
  CDR
IYWDDDK

SEQ ID NO: 463 - protein, 15 aa, Artificial sequence
  CDR
ARAPPPNWDEYYFDY

SEQ ID NO: 464 - protein, 7 aa, Artificial sequence
  CDR
TSGMGVS

SEQ ID NO: 465 - protein, 16 aa, Artificial sequence
  CDR
HIYWDDDKRYNPSLKS

SEQ ID NO: 466 - protein, 13 aa, Artificial sequence
  CDR
APPPNWDEYYFDY

SEQ ID NO: 467 - protein, 9 aa, Artificial sequence
  CDR
GFSLSTSGM

SEQ ID NO: 468 - protein, 5 aa, Artificial sequence
  CDR
YWDDD

SEQ ID NO: 469 - protein, 13 aa, Artificial sequence
  CDR
APPPNWDEYYFDY

SEQ ID NO: 470 - protein, 6 aa, Artificial sequence
  CDR
GNIHNS

SEQ ID NO: 471 - protein, 2 aa, Artificial sequence
  CDR
NA

SEQ ID NO: 472 - protein, 8 aa, Artificial sequence
  CDR
QHFWSTPP

SEQ ID NO: 473 - protein, 11 aa, Artificial sequence
  CDR
RASGNIHNSLA

SEQ ID NO: 474 - protein, 7 aa, Artificial sequence
  CDR
NAKTLAD

SEQ ID NO: 475 - protein, 8 aa, Artificial sequence
  CDR
QHFWSTPP

SEQ ID NO: 476 - protein, 11 aa, Artificial sequence
  CDR
RASGNIHNSLA

SEQ ID NO: 477 - protein, 7 aa, Artificial sequence
  CDR
NAKTLAD

SEQ ID NO: 478 - protein, 8 aa, Artificial sequence
  CDR
QHFWSTPP

SEQ ID NO: 479 - protein, 8 aa, Artificial sequence
  CDR
GFSLSRYT

SEQ ID NO: 480 - protein, 7 aa, Artificial sequence
  CDR
IWGGGST

SEQ ID NO: 481 - protein, 13 aa, Artificial sequence
  CDR
ARPHDFDAGGFAY

SEQ ID NO: 482 - protein, 5 aa, Artificial sequence
  CDR
RYTVH

SEQ ID NO: 483 - protein, 16 aa, Artificial sequence
  CDR
MIWGGGSTDYNSALKS

SEQ ID NO: 484 - protein, 11 aa, Artificial sequence
  CDR
PHDFDAGGFAY

SEQ ID NO: 485 - protein, 7 aa, Artificial sequence
  CDR
GFSLSRY

SEQ ID NO: 486 - protein, 5 aa, Artificial sequence
  CDR
WGGGS

SEQ ID NO: 487 - protein, 11 aa, Artificial sequence
  CDR
PHDFDAGGFAY

SEQ ID NO: 488 - protein, 5 aa, Artificial sequence
  CDR
SRVNY

SEQ ID NO: 489 - protein, 2 aa, Artificial sequence
  CDR
DT

SEQ ID NO: 490 - protein, 9 aa, Artificial sequence
  CDR
QQWTSYPLT

SEQ ID NO: 491 - protein, 10 aa, Artificial sequence
  CDR
SASSRVNYMY

SEQ ID NO: 492 - protein, 7 aa, Artificial sequence
  CDR
DTSNLAS

SEQ ID NO: 493 - protein, 9 aa, Artificial sequence
  CDR
QQWTSYPLT

SEQ ID NO: 494 - protein, 10 aa, Artificial sequence
  CDR
SASSRVNYMY

SEQ ID NO: 495 - protein, 7 aa, Artificial sequence
  CDR
DTSNLAS

SEQ ID NO: 496 - protein, 9 aa, Artificial sequence
  CDR
QQWTSYPLT

SEQ ID NO: 497 - protein, 8 aa, Artificial sequence
  CDR
GYTFRDYW

SEQ ID NO: 498 - protein, 8 aa, Artificial sequence
  CDR
ILPGSGNA

SEQ ID NO: 499 - protein, 11 aa, Artificial sequence
  CDR
ARVYSNWYFDA

SEQ ID NO: 500 - protein, 5 aa, Artificial sequence
  CDR
DYWLE

SEQ ID NO: 501 - protein, 17 aa, Artificial sequence
  CDR
EILPGSGNAYYNEKFKG

SEQ ID NO: 502 - protein, 9 aa, Artificial sequence
  CDR
VYSNWYFDA

SEQ ID NO: 503 - protein, 7 aa, Artificial sequence
  CDR
GYTFRDY

SEQ ID NO: 504 - protein, 6 aa, Artificial sequence
  CDR
LPGSGN

SEQ ID NO: 505 - protein, 9 aa, Artificial sequence
  CDR
VYSNWYFDA

SEQ ID NO: 506 - protein, 9 aa, Artificial sequence
  CDR
TGAVTTSNY

SEQ ID NO: 507 - protein, 2 aa, Artificial sequence
  CDR
GT

SEQ ID NO: 508 - protein, 9 aa, Artificial sequence
  CDR
ALWFSNHWV

SEQ ID NO: 509 - protein, 14 aa, Artificial sequence
  CDR
RSSTGAVTTSNYAN

SEQ ID NO: 510 - protein, 7 aa, Artificial sequence
  CDR
GTNNQAP

SEQ ID NO: 511 - protein, 9 aa, Artificial sequence
  CDR
ALWFSNHWV

SEQ ID NO: 512 - protein, 14 aa, Artificial sequence
  CDR
RSSTGAVTTSNYAN

SEQ ID NO: 513 - protein, 7 aa, Artificial sequence
  CDR
GTNNQAP

SEQ ID NO: 514 - protein, 9 aa, Artificial sequence
  CDR
ALWFSNHWV

SEQ ID NO: 515 - protein, 8 aa, Artificial sequence
  CDR
GFTFSDYG

SEQ ID NO: 516 - protein, 8 aa, Artificial sequence
  CDR
ISRGSHTI

SEQ ID NO: 517 - protein, 11 aa, Artificial sequence
  CDR
VRMAGYYAMDY

SEQ ID NO: 518 - protein, 5 aa, Artificial sequence
  CDR
DYGMN

SEQ ID NO: 519 - protein, 17 aa, Artificial sequence
  CDR
YISRGSHTIYYADTVKG

SEQ ID NO: 520 - protein, 9 aa, Artificial sequence
  CDR
MAGYYAMDY

SEQ ID NO: 521 - protein, 7 aa, Artificial sequence
  CDR
GFTFSDY

SEQ ID NO: 522 - protein, 6 aa, Artificial sequence
  CDR
SRGSHT

SEQ ID NO: 523 - protein, 9 aa, Artificial sequence
  CDR
MAGYYAMDY

SEQ ID NO: 524 - protein, 7 aa, Artificial sequence
  CDR
SQHSSYI

SEQ ID NO: 525 - protein, 6 aa, Artificial sequence
  CDR
LKKDGS

SEQ ID NO: 526 - protein, 13 aa, Artificial sequence
  CDR
GVGDTIKEQFVYV

SEQ ID NO: 527 - protein, 12 aa, Artificial sequence
  CDR
TLSSQHSSYIIE

SEQ ID NO: 528 - protein, 11 aa, Artificial sequence
  CDR
LKKDGSHSTGD

SEQ ID NO: 529 - protein, 13 aa, Artificial sequence
  CDR
GVGDTIKEQFVYV

SEQ ID NO: 530 - protein, 12 aa, Artificial sequence
  CDR
TLSSQHSSYIIE

SEQ ID NO: 531 - protein, 11 aa, Artificial sequence
  CDR
LKKDGSHSTGD

SEQ ID NO: 532 - protein, 13 aa, Artificial sequence
  CDR
GVGDTIKEQFVYV

SEQ ID NO: 533 - protein, 8 aa, Artificial sequence
  CDR
GFTFSDYG

SEQ ID NO: 534 - protein, 8 aa, Artificial sequence
  CDR
ISRGSHTI

SEQ ID NO: 535 - protein, 11 aa, Artificial sequence
  CDR
VRMAGYYAMDY

SEQ ID NO: 536 - protein, 5 aa, Artificial sequence
  CDR
DYGMN

SEQ ID NO: 537 - protein, 17 aa, Artificial sequence
  CDR
YISRGSHTIYYADTVKG

SEQ ID NO: 538 - protein, 9 aa, Artificial sequence
  CDR
MAGYYAMDY

SEQ ID NO: 539 - protein, 7 aa, Artificial sequence
  CDR
GFTFSDY

SEQ ID NO: 540 - protein, 6 aa, Artificial sequence
  CDR
SRGSHT

SEQ ID NO: 541 - protein, 9 aa, Artificial sequence
  CDR
MAGYYAMDY

SEQ ID NO: 542 - protein, 7 aa, Artificial sequence
  CDR
SQHSSYI

SEQ ID NO: 543 - protein, 6 aa, Artificial sequence
  CDR
VKKDGS

SEQ ID NO: 544 - protein, 13 aa, Artificial sequence
  CDR
GVGDTIKEQFVYV

SEQ ID NO: 545 - protein, 12 aa, Artificial sequence
  CDR
TLSSQHSSYIIE

SEQ ID NO: 546 - protein, 11 aa, Artificial sequence
  CDR
VKKDGSHSTGD

SEQ ID NO: 547 - protein, 13 aa, Artificial sequence
  CDR
GVGDTIKEQFVYV

SEQ ID NO: 548 - protein, 12 aa, Artificial sequence
  CDR
TLSSQHSSYIIE

SEQ ID NO: 549 - protein, 11 aa, Artificial sequence
  CDR
VKKDGSHSTGD

SEQ ID NO: 550 - protein, 13 aa, Artificial sequence
  CDR
GVGDTIKEQFVYV

SEQ ID NO: 551 - protein, 8 aa, Artificial sequence
  CDR
GFTFSDYY

SEQ ID NO: 552 - protein, 8 aa, Artificial sequence
  CDR
ISNTGYST

SEQ ID NO: 553 - protein, 11 aa, Artificial sequence
  CDR
ARHGILYAMDY

SEQ ID NO: 554 - protein, 5 aa, Artificial sequence
  CDR
DYYMY

SEQ ID NO: 555 - protein, 17 aa, Artificial sequence
  CDR
YISNTGYSTFYPDSVKG

SEQ ID NO: 556 - protein, 9 aa, Artificial sequence
  CDR
HGILYAMDY

SEQ ID NO: 557 - protein, 7 aa, Artificial sequence
  CDR
GFTFSDY

SEQ ID NO: 558 - protein, 6 aa, Artificial sequence
  CDR
SNTGYS

SEQ ID NO: 559 - protein, 9 aa, Artificial sequence
  CDR
HGILYAMDY

SEQ ID NO: 560 - protein, 7 aa, Artificial sequence
  CDR
SQYSTYT

SEQ ID NO: 561 - protein, 6 aa, Artificial sequence
  CDR
LKHDGS

SEQ ID NO: 562 - protein, 13 aa, Artificial sequence
  CDR
GVGDTIKEQFMYV

SEQ ID NO: 563 - protein, 12 aa, Artificial sequence
  CDR
TLSSQYSTYTIG

SEQ ID NO: 564 - protein, 11 aa, Artificial sequence
  CDR
LKHDGSRHTGD

SEQ ID NO: 565 - protein, 13 aa, Artificial sequence
  CDR
GVGDTIKEQFMYV

SEQ ID NO: 566 - protein, 12 aa, Artificial sequence
  CDR
TLSSQYSTYTIG

SEQ ID NO: 567 - protein, 11 aa, Artificial sequence
  CDR
LKHDGSRHTGD

SEQ ID NO: 568 - protein, 13 aa, Artificial sequence
  CDR
GVGDTIKEQFMYV

SEQ ID NO: 569 - protein, 8 aa, Artificial sequence
  CDR
GFTFSDYV

SEQ ID NO: 570 - protein, 8 aa, Artificial sequence
  CDR
ISDAGSYT

SEQ ID NO: 571 - protein, 11 aa, Artificial sequence
  CDR
AIIYFGNYGGY

SEQ ID NO: 572 - protein, 5 aa, Artificial sequence
  CDR
DYVMS

SEQ ID NO: 573 - protein, 17 aa, Artificial sequence
  CDR
TISDAGSYTFYPDNLKG

SEQ ID NO: 574 - protein, 9 aa, Artificial sequence
  CDR
IYFGNYGGY

SEQ ID NO: 575 - protein, 7 aa, Artificial sequence
  CDR
GFTFSDY

SEQ ID NO: 576 - protein, 6 aa, Artificial sequence
  CDR
SDAGSY

SEQ ID NO: 577 - protein, 9 aa, Artificial sequence
  CDR
IYFGNYGGY

SEQ ID NO: 578 - protein, 9 aa, Artificial sequence
  CDR
TGAVTTSNY

SEQ ID NO: 579 - protein, 2 aa, Artificial sequence
  CDR
GT

SEQ ID NO: 580 - protein, 9 aa, Artificial sequence
  CDR
ALWYSTHWV

SEQ ID NO: 581 - protein, 14 aa, Artificial sequence
  CDR
RSNTGAVTTSNYAN

SEQ ID NO: 582 - protein, 7 aa, Artificial sequence
  CDR
GTSNRAP

SEQ ID NO: 583 - protein, 9 aa, Artificial sequence
  CDR
ALWYSTHWV

SEQ ID NO: 584 - protein, 14 aa, Artificial sequence
  CDR
RSNTGAVTTSNYAN

SEQ ID NO: 585 - protein, 7 aa, Artificial sequence
  CDR
GTSNRAP

SEQ ID NO: 586 - protein, 9 aa, Artificial sequence
  CDR
ALWYSTHWV

SEQ ID NO: 587 - protein, 8 aa, Artificial sequence
  CDR
GFTFSNYA

SEQ ID NO: 588 - protein, 8 aa, Artificial sequence
  CDR
ISSIGSFT

SEQ ID NO: 589 - protein, 9 aa, Artificial sequence
  CDR
ARLGVYFDS

SEQ ID NO: 590 - protein, 5 aa, Artificial sequence
  CDR
NYAMS

SEQ ID NO: 591 - protein, 17 aa, Artificial sequence
  CDR
TISSIGSFTYYSDSVKG

SEQ ID NO: 592 - protein, 7 aa, Artificial sequence
  CDR
LGVYFDS

SEQ ID NO: 593 - protein, 7 aa, Artificial sequence
  CDR
GFTFSNY

SEQ ID NO: 594 - protein, 6 aa, Artificial sequence
  CDR
SSIGSF

SEQ ID NO: 595 - protein, 7 aa, Artificial sequence
  CDR
LGVYFDS

SEQ ID NO: 596 - protein, 7 aa, Artificial sequence
  CDR
SQHSTYT

SEQ ID NO: 597 - protein, 6 aa, Artificial sequence
  CDR
IKKDGS

SEQ ID NO: 598 - protein, 13 aa, Artificial sequence
  CDR
GVSDMIKDQFVYV

SEQ ID NO: 599 - protein, 12 aa, Artificial sequence
  CDR
TLSSQHSTYTIE

SEQ ID NO: 600 - protein, 11 aa, Artificial sequence
  CDR
IKKDGSHYTGD

SEQ ID NO: 601 - protein, 13 aa, Artificial sequence
  CDR
GVSDMIKDQFVYV

SEQ ID NO: 602 - protein, 12 aa, Artificial sequence
  CDR
TLSSQHSTYTIE

SEQ ID NO: 603 - protein, 11 aa, Artificial sequence
  CDR
IKKDGSHYTGD

SEQ ID NO: 604 - protein, 13 aa, Artificial sequence
  CDR
GVSDMIKDQFVYV

SEQ ID NO: 605 - protein, 8 aa, Artificial sequence
  CDR
GFSLTGYG

SEQ ID NO: 606 - protein, 7 aa, Artificial sequence
  CDR
IWGGGGT

SEQ ID NO: 607 - protein, 11 aa, Artificial sequence
  CDR
ARYSKYGHFDV

SEQ ID NO: 608 - protein, 5 aa, Artificial sequence
  CDR
GYGVQ

SEQ ID NO: 609 - protein, 16 aa, Artificial sequence
  CDR
VIWGGGGTDYNAAFIS

SEQ ID NO: 610 - protein, 9 aa, Artificial sequence
  CDR
YSKYGHFDV

SEQ ID NO: 611 - protein, 7 aa, Artificial sequence
  CDR
GFSLTGY

SEQ ID NO: 612 - protein, 5 aa, Artificial sequence
  CDR
WGGGG

SEQ ID NO: 613 - protein, 9 aa, Artificial sequence
  CDR
YSKYGHFDV

SEQ ID NO: 614 - protein, 9 aa, Artificial sequence
  CDR
TGAVTTSNY

SEQ ID NO: 615 - protein, 2 aa, Artificial sequence
  CDR
GT

SEQ ID NO: 616 - protein, 10 aa, Artificial sequence
  CDR
ALWYSNHFWV

SEQ ID NO: 617 - protein, 14 aa, Artificial sequence
  CDR
RSSTGAVTTSNYAN

SEQ ID NO: 618 - protein, 7 aa, Artificial sequence
  CDR
GTYNRVP

SEQ ID NO: 619 - protein, 10 aa, Artificial sequence
  CDR
ALWYSNHFWV

SEQ ID NO: 620 - protein, 14 aa, Artificial sequence
  CDR
RSSTGAVTTSNYAN

SEQ ID NO: 621 - protein, 7 aa, Artificial sequence
  CDR
GTYNRVP

SEQ ID NO: 622 - protein, 10 aa, Artificial sequence
  CDR
ALWYSNHFWV

SEQ ID NO: 623 - protein, 10 aa, Artificial sequence
  CDR
GFSLSTSGMG

SEQ ID NO: 624 - protein, 7 aa, Artificial sequence
  CDR
IWWDDDE

SEQ ID NO: 625 - protein, 16 aa, Artificial sequence
  CDR
ARISIPYGYYDWFFDV

SEQ ID NO: 626 - protein, 7 aa, Artificial sequence
  CDR
TSGMGVG

SEQ ID NO: 627 - protein, 16 aa, Artificial sequence
  CDR
HIWWDDDEYYNPALKS

SEQ ID NO: 628 - protein, 14 aa, Artificial sequence
  CDR
ISIPYGYYDWFFDV

SEQ ID NO: 629 - protein, 9 aa, Artificial sequence
  CDR
GFSLSTSGM

SEQ ID NO: 630 - protein, 5 aa, Artificial sequence
  CDR
WWDDD

SEQ ID NO: 631 - protein, 14 aa, Artificial sequence
  CDR
ISIPYGYYDWFFDV

SEQ ID NO: 632 - protein, 9 aa, Artificial sequence
  CDR
TGAVTTRNY

SEQ ID NO: 633 - protein, 2 aa, Artificial sequence
  CDR
GT

SEQ ID NO: 634 - protein, 9 aa, Artificial sequence
  CDR
ALWYSNHFI

SEQ ID NO: 635 - protein, 14 aa, Artificial sequence
  CDR
RSSTGAVTTRNYAN

SEQ ID NO: 636 - protein, 7 aa, Artificial sequence
  CDR
GTNNRAP

SEQ ID NO: 637 - protein, 9 aa, Artificial sequence
  CDR
ALWYSNHFI

SEQ ID NO: 638 - protein, 14 aa, Artificial sequence
  CDR
RSSTGAVTTRNYAN

SEQ ID NO: 639 - protein, 7 aa, Artificial sequence
  CDR
GTNNRAP

SEQ ID NO: 640 - protein, 9 aa, Artificial sequence
  CDR
ALWYSNHFI

SEQ ID NO: 641 - protein, 10 aa, Artificial sequence
  CDR
GFSLSTFPMG

SEQ ID NO: 642 - protein, 7 aa, Artificial sequence
  CDR
IWWDDDE

SEQ ID NO: 643 - protein, 12 aa, Artificial sequence
  CDR
TRANWDRWYFDV

SEQ ID NO: 644 - protein, 7 aa, Artificial sequence
  CDR
TFPMGVG

SEQ ID NO: 645 - protein, 16 aa, Artificial sequence
  CDR
HIWWDDDEYYNPVLKS

SEQ ID NO: 646 - protein, 10 aa, Artificial sequence
  CDR
ANWDRWYFDV

SEQ ID NO: 647 - protein, 9 aa, Artificial sequence
  CDR
GFSLSTFPM

SEQ ID NO: 648 - protein, 5 aa, Artificial sequence
  CDR
WWDDD

SEQ ID NO: 649 - protein, 10 aa, Artificial sequence
  CDR
ANWDRWYFDV

SEQ ID NO: 650 - protein, 7 aa, Artificial sequence
  CDR
RQHSAYT

SEQ ID NO: 651 - protein, 6 aa, Artificial sequence
  CDR
VKKDGS

SEQ ID NO: 652 - protein, 11 aa, Artificial sequence
  CDR
GVGDTIKEHFV

SEQ ID NO: 653 - protein, 12 aa, Artificial sequence
  CDR
TLSRQHSAYTIE

SEQ ID NO: 654 - protein, 11 aa, Artificial sequence
  CDR
VKKDGSHSTGD

SEQ ID NO: 655 - protein, 11 aa, Artificial sequence
  CDR
GVGDTIKEHFV

SEQ ID NO: 656 - protein, 12 aa, Artificial sequence
  CDR
TLSRQHSAYTIE

SEQ ID NO: 657 - protein, 11 aa, Artificial sequence
  CDR
VKKDGSHSTGD

SEQ ID NO: 658 - protein, 11 aa, Artificial sequence
  CDR
GVGDTIKEHFV

SEQ ID NO: 659 - protein, 10 aa, Artificial sequence
  CDR
GFSLSTFGMG

SEQ ID NO: 660 - protein, 7 aa, Artificial sequence
  CDR
IWWDDDK

SEQ ID NO: 661 - protein, 11 aa, Artificial sequence
  CDR
ARDNWVSSLDY

SEQ ID NO: 662 - protein, 7 aa, Artificial sequence
  CDR
TFGMGVG

SEQ ID NO: 663 - protein, 16 aa, Artificial sequence
  CDR
HIWWDDDKHYNPALKS

SEQ ID NO: 664 - protein, 9 aa, Artificial sequence
  CDR
DNWVSSLDY

SEQ ID NO: 665 - protein, 9 aa, Artificial sequence
  CDR
GFSLSTFGM

SEQ ID NO: 666 - protein, 5 aa, Artificial sequence
  CDR
WWDDD

SEQ ID NO: 667 - protein, 9 aa, Artificial sequence
  CDR
DNWVSSLDY

SEQ ID NO: 668 - protein, 7 aa, Artificial sequence
  CDR
SQHSTYT

SEQ ID NO: 669 - protein, 6 aa, Artificial sequence
  CDR
LKKDGS

SEQ ID NO: 670 - protein, 13 aa, Artificial sequence
  CDR
GVSDTIKEQFVYV

SEQ ID NO: 671 - protein, 12 aa, Artificial sequence
  CDR
TLSSQHSTYTIE

SEQ ID NO: 672 - protein, 11 aa, Artificial sequence
  CDR
LKKDGSHSTGD

SEQ ID NO: 673 - protein, 13 aa, Artificial sequence
  CDR
GVSDTIKEQFVYV

SEQ ID NO: 674 - protein, 12 aa, Artificial sequence
  CDR
TLSSQHSTYTIE

SEQ ID NO: 675 - protein, 11 aa, Artificial sequence
  CDR
LKKDGSHSTGD

SEQ ID NO: 676 - protein, 13 aa, Artificial sequence
  CDR
GVSDTIKEQFVYV

SEQ ID NO: 677 - protein, 10 aa, Artificial sequence
  CDR
GFSLSTFPLG

SEQ ID NO: 678 - protein, 7 aa, Artificial sequence
  CDR
VWWNDDD

SEQ ID NO: 679 - protein, 12 aa, Artificial sequence
  CDR
ARLNWDRWYFDV

SEQ ID NO: 680 - protein, 7 aa, Artificial sequence
  CDR
TFPLGVG

SEQ ID NO: 681 - protein, 16 aa, Artificial sequence
  CDR
HVWWNDDDYYNPALKS

SEQ ID NO: 682 - protein, 10 aa, Artificial sequence
  CDR
LNWDRWYFDV

SEQ ID NO: 683 - protein, 9 aa, Artificial sequence
  CDR
GFSLSTFPL

SEQ ID NO: 684 - protein, 5 aa, Artificial sequence
  CDR
WWNDD

SEQ ID NO: 685 - protein, 10 aa, Artificial sequence
  CDR
LNWDRWYFDV

SEQ ID NO: 686 - protein, 7 aa, Artificial sequence
  CDR
SQHSAYT

SEQ ID NO: 687 - protein, 6 aa, Artificial sequence
  CDR
LKKDGS

SEQ ID NO: 688 - protein, 11 aa, Artificial sequence
  CDR
GVGDTVKEQFV

SEQ ID NO: 689 - protein, 12 aa, Artificial sequence
  CDR
TLSSQHSAYTVE

SEQ ID NO: 690 - protein, 11 aa, Artificial sequence
  CDR
LKKDGSHSTGD

SEQ ID NO: 691 - protein, 11 aa, Artificial sequence
  CDR
GVGDTVKEQFV

SEQ ID NO: 692 - protein, 12 aa, Artificial sequence
  CDR
TLSSQHSAYTVE

SEQ ID NO: 693 - protein, 11 aa, Artificial sequence
  CDR
LKKDGSHSTGD

SEQ ID NO: 694 - protein, 11 aa, Artificial sequence
  CDR
GVGDTVKEQFV

SEQ ID NO: 695 - protein, 10 aa, Artificial sequence
  CDR
GFSLSTFDRG

SEQ ID NO: 696 - protein, 7 aa, Artificial sequence
  CDR
IWWNDKK

SEQ ID NO: 697 - protein, 12 aa, Artificial sequence
  CDR
ARIVWDKYYFDY

SEQ ID NO: 698 - protein, 7 aa, Artificial sequence
  CDR
TFDRGVG

SEQ ID NO: 699 - protein, 16 aa, Artificial sequence
  CDR
HIWWNDKKFYNPVLKS

SEQ ID NO: 700 - protein, 10 aa, Artificial sequence
  CDR
IVWDKYYFDY

SEQ ID NO: 701 - protein, 9 aa, Artificial sequence
  CDR
GFSLSTFDR

SEQ ID NO: 702 - protein, 5 aa, Artificial sequence
  CDR
WWNDK

SEQ ID NO: 703 - protein, 10 aa, Artificial sequence
  CDR
IVWDKYYFDY

SEQ ID NO: 704 - protein, 7 aa, Artificial sequence
  CDR
SQHSAYT

SEQ ID NO: 705 - protein, 6 aa, Artificial sequence
  CDR
LKKDGS

SEQ ID NO: 706 - protein, 11 aa, Artificial sequence
  CDR
GVGDTIKEQYV

SEQ ID NO: 707 - protein, 12 aa, Artificial sequence
  CDR
TLSSQHSAYTIE

SEQ ID NO: 708 - protein, 11 aa, Artificial sequence
  CDR
LKKDGSHSTGD

SEQ ID NO: 709 - protein, 11 aa, Artificial sequence
  CDR
GVGDTIKEQYV

SEQ ID NO: 710 - protein, 12 aa, Artificial sequence
  CDR
TLSSQHSAYTIE

SEQ ID NO: 711 - protein, 11 aa, Artificial sequence
  CDR
LKKDGSHSTGD

SEQ ID NO: 712 - protein, 11 aa, Artificial sequence
  CDR
GVGDTIKEQYV

(19) United States
(12) Patent Application Publication
(10) Pub. No.: US 2024/0084003 A1
(43) Pub. Date: Mar. 14, 2024

(54) ANTI-HUMAN CD22 MONOCLONAL ANTIBODIES AND USE THEREOF

(71) Applicant: Simcere Zaiming Pharmaceutical Co., Ltd., Hainan (CN)

(72) Inventors: Qiong Wang, Shanghai (CN); Cuiqing Yang, Shanghai (CN); Zhuoxiao Cao, Shanghai (CN); Renhong Tang, Shanghai (CN); Jinsheng Ren, Jiangsu (CN)

(21) Appl. No.: 18/272,539

(22) PCT Filed: Jan. 17, 2022
(86) PCT No.: PCT/CN2022/072234

§ 371 Date: Jul. 14, 2023

(30) Foreign Application Priority Data
Jan. 18, 2021 (CN) 202110060035.4

(51) Int. Cl. C07K 16/28; A61K 39/00; A61P 35/00; C12N 15/63

(52) U.S. Cl. CPC C07K 16/2803; A61K 39/4631; A61P 35/00; C12N 15/63; A61K 2239/13; A61K 2239/29; A61K 2239/48; C07K 2317/14; C07K 2317/24; C07K 2317/31; C07K 2317/565; C07K 2317/567; C07K 2317/92

(57) ABSTRACT

CD22 antibodies, a preparation method therefor, and an application thereof. The CD22 antibodies have a high affinity to CD22 protein Therefore, the CD22 antibodies can be used in the preparation of drugs for the treatment of diseases such as tumors and autoimmune diseases.

The present application claims the right of priority of Chinese patent application no. 202110060035.4 titled “ANTI-HUMAN CD22 MONOCLONAL ANTIBODIES AND USE THEREOF” and filed with the China Patent Office on Monday, Jan. 18, 2021, which is incorporated in the present disclosure by reference in its entirety.

### TECHNICAL FIELD

The present invention relates to the fields of bioengineering and biomedicine, and mainly relates to a monoclonal antibody targeting human CD22 or an antigen-binding fragment thereof, and an encoding nucleic acid, an expression vector and an expression cell, a preparation method, a pharmaceutical composition therefor, and their use for treating diseases, such as use for treating tumors and autoimmune diseases.

### BACKGROUND ART

CD22 is a type I transmembrane glycoprotein, which belongs to the sialic acid-binding immunoglobulinlike lectins (Siglec) family. As a B lineage differentiation antigen, it is specifically expressed in B cells. The expression of CD22 begins from the pre-B cell (pre-B cell) stage, and stops after B cells differentiate into plasma cells. The broad-spectrum expression of CD22 in B cell development makes it an attractive molecule for targeting B cells.

The extracellular region of CD22 consists of 7 Ig-like domains and 12 predicted N-linked glycosylation sites, and its N-terminal (i.e., distal end of membrane) domain 1 is V Type Ig-like domain, which, as a ligand binding site, can recognize α2,6-coupled sialic acid. The intracellular region of CD22 has immunoreceptor tyrosine-based inhibitory motifs (ITIMs). When the tyrosine on the ITIMs is phosphorylated by the Src family protein kinase, binding sites for molecules containing SH2 (Src homology2) domain would be generated, then SHP-1 (Src homology region 2 domain-containing phosphatase-1) was recruited to inhibit the BCR (B-cell receptor) signaling pathway of normal B cells.

α2,6-coupled sialoglycoprotein exists in hematopoietic cells, some endothelial cells, T cells and B cells, and CD22 protein itself also produces α2,6-coupled sialic acid, so CD22 can form cis-interaction with itself and other sialoglycoproteins on the surface of B cells, and trans-interaction with sialoglycoproteins on the surface of other types of cells. In resting B cells, the cis-interaction between CD22 molecules makes the ligand-binding site of CD22 masked, but once the ligand is presented by an adjacent cell, the masked ligand-binding site of CD22 is exposed and interacts with the ligand of the adjacent cells to form trans-interaction. The cis-interaction between CD22 molecules forms homo-oligomers on the B cell surface and the homo-oligomers can form a dynamic nanocluster and generate an antigen binding signal threshold that must be reached before B cell activation, thereby regulating B cell signaling pathway.

CD22 is expressed in 60% to 90% of B cell malignancies and is not expressed in hematopoietic stem cells. In an early clinical study on acute lymphoblastic leukemia (ALL), CD22 is expressed in 60% to 85% of ALL. In another study, the positive rate of CD22 in B-lineage ALL patients reaches 93%. CD22 is expressed in more than 85% of patients with diffuse large B-cell lymphomas (DLBCLs). There are many clinical trials investigating the effectiveness of drugs that target CD22. Epratuzumab is a CD22 monoclonal antibody that has certain effects in adults and children with B-ALL. CD22 antibody-conjugated drugs have a certain therapeutic effect on B-ALL.

Monoclonal antibodies, due to their advantages such as targeting ability, specificity, selectivity, high affinity, etc., are becoming a new type of therapeutic drug. However, early clinical trials revealed that the use of non-human monoclonal antibodies in humans often resulted in severe immune responses due to human anti-mouse antibody (HAMA) and human anti-rat antibody (HARA) responses, and the antibodies were rapidly cleared. Less immunogenic antibodies were subsequently developed, including chimeric, humanized, and fully human antibodies. According to the degree of humanization, therapeutic monoclonal antibody drugs can be divided into 4 types: a murine antibody (without human amino acid sequence), a chimeric antibody (with 60%-70% humanized amino acid sequence), a CDR-grafted antibody (with 90%-95% humanized amino acid sequence) and a fully human antibody (with 100% human amino acid sequence). As the degree of humanization increases, a non-murine monoclonal antibody can reduce human anti-mouse antibody reactions (HAMA and HARA reactions) during human treatment, gradually eliminate the immunogenicity of a heterologous antibody, and maintain high affinity to an antigen and improve pharmacokinetics of the antibody at the same time, and these antibody drugs have been widely used in clinical targeted therapy.

### SUMMARY OF THE INVENTION

The present invention provides an antibody or an antigen-binding fragment that specifically binds to human CD22, a nucleic acid encoding the antibody and the antigen-binding fragment, a pharmaceutical composition and a kit comprising the antibody and the antigen-binding fragment, and their use in the preparation of drugs for treating tumors, etc.

In some embodiments, the antibody or the antigen-binding fragment that specifically binds to human CD22 comprises a combination of CDRs comprising: CDR1, CDR2, and CDR3; the CDR1, CDR2 and CDR3 have any sequence combination selected from the following, or a sequence combination with 1, 2, 3 or more amino acid insertions, deletions and/or substitutions compared to the sequence combination:

| | SEQ ID NO. | | |
|---|---|---|---|
| No. | CDR1-VH | CDR2-VH | CDR3-VH |
| VH1 | SEQ ID NO. 83 | SEQ ID NO. 84 | SEQ ID NO. 85 |
| VH2 | SEQ ID NO. 86 | SEQ ID NO. 87 | SEQ ID NO. 88 |
| VH3 | SEQ ID NO. 89 | SEQ ID NO. 90 | SEQ ID NO. 91 |
| VH4 | SEQ ID NO. 101 | SEQ ID NO. 102 | SEQ ID NO. 103 |
| VH5 | SEQ ID NO. 104 | SBQ ID NO. 105 | SEQ ID NO. 106 |
| VH6 | SEQ ID NO. 107 | SEQ ID NO. 108 | SEQ ID NO. 109 |
| VH7 | SEQ ID NO. 119 | SEQ ID NO. 120 | SEQ ID NO. 121 |
| VH8 | SEQ ID NO. 122 | SEQ ID NO. 123 | SEQ ID NO. 124 |
| VH9 | SEQ ID NO. 125 | SEQ ID NO. 126 | SEQ ID NO. 127 |
| VH10 | SEQ ID NO. 137 | SEQ ID NO. 138 | SEQ ID NO. 139 |
| VH11 | SEQ ID NO. 140 | SEQ ID NO. 141 | SEQ ID NO. 142 |
| VH12 | SEQ ID NO. 143 | SEQ ID NO. 144 | SEQ ID NO. 145 |
| VH13 | SEQ ID NO. 155 | SEQ ID NO. 156 | SEQ ID NO. 157 |
| VH14 | SEQ ID NO. 158 | SBQ ID NO. 159 | SEQ ID NO. 160 |
| VH15 | SEQ ID NO. 161 | SEQ ID NO. 162 | SEQ ID NO. 163 |
| VH16 | SEQ ID NO. 173 | SEQ ID NO. 174 | SEQ ID NO. 175 |
| VH17 | SEQ ID NO. 176 | SEQ ID NO. 177 | SEQ ID NO. 178 |
| VH18 | SEQ ID NO. 179 | SEQ ID NO. 180 | SEQ ID NO. 181 |
| VH19 | SEQ ID NO. 191 | SEQ ID NO. 192 | SEQ ID NO. 193 |
| VH20 | SEQ ID NO. 194 | SEQ ID NO. 195 | SEQ ID NO. 196 |
| VH21 | SEQ ID NO. 197 | SEQ ID NO. 198 | SEQ ID NO. 199 |
| VH22 | SEQ ID NO. 209 | SEQ ID NO. 210 | SEQ ID NO. 211 |
| VH23 | SEQ ID NO. 212 | SEQ ID NO. 213 | SEQ ID NO. 214 |
| VH24 | SEQ ID NO. 215 | SEQ ID NO. 216 | SEQ ID NO. 217 |
| VH25 | SEQ ID NO. 227 | SEQ ID NO. 228 | SEQ ID NO. 229 |
| VH26 | SEQ ID NO. 230 | SEQ ID NO. 231 | SEQ ID NO. 232 |
| VH27 | SEQ ID NO. 233 | SEQ ID NO. 234 | SEQ ID NO. 235 |
| VH28 | SEQ ID NO. 245 | SEQ ID NO. 246 | SEQ ID NO. 247 |
| VH29 | SEQ ID NO. 248 | SEQ ID NO. 249 | SEQ ID NO. 250 |
| VH30 | SEQ ID NO. 251 | SEQ ID NO. 252 | SEQ ID NO. 253 |
| VH31 | SEQ ID NO. 263 | SEQ ID NO. 264 | SEQ ID NO. 265 |
| VH32 | SEQ ID NO. 266 | SEQ ID NO. 267 | SEQ ID NO. 268 |
| VH33 | SEQ ID NO. 269 | SEQ ID NO. 270 | SEQ ID NO. 271 |
| VH34 | SEQ ID NO. 281 | SEQ ID NO. 282 | SEQ ID NO. 283 |
| VH35 | SEQ ID NO. 284 | SEQ ID NO. 285 | SEQ ID NO. 286 |
| VH36 | SEQ ID NO. 287 | SEQ ID NO. 288 | SEQ ID NO. 289 |
| VH37 | SEQ ID NO. 299 | SEQ ID NO. 300 | SEQ ID NO. 301 |
| VH38 | SEQ ID NO. 302 | SEQ ID NO. 303 | SEQ ID NO. 304 |
| VH39 | SEQ ID NO. 305 | SEQ ID NO. 306 | SEQ ID NO. 307 |
| VH40 | SEQ ID NO. 317 | SEQ ID NO. 318 | SEQ ID NO. 319 |
| VH41 | SEQ ID NO. 320 | SEQ ID NO. 321 | SEQ ID NO. 322 |
| VH42 | SEQ ID NO. 323 | SEQ ID NO. 324 | SEQ ID NO. 325 |
| VH43 | SEQ ID NO. 335 | SEQ ID NO. 336 | SEQ ID NO. 337 |
| VH44 | SEQ ID NO. 338 | SEQ ID NO. 339 | SEQ ID NO. 340 |
| VH45 | SEQ ID NO. 341 | SEQ ID NO. 342 | SEQ ID NO. 343 |
| VH46 | SEQ ID NO. 353 | SEQ ID NO. 354 | SEQ ID NO. 355 |
| VH47 | SEQ ID NO. 356 | SEQ ID NO. 357 | SEQ ID NO. 358 |
| VH48 | SEQ ID NO. 359 | SEQ ID NO. 360 | SEQ ID NO. 361 |
| VH49 | SEQ ID NO. 371 | SEQ ID NO. 372 | SEQ ID NO. 373 |
| VH50 | SEQ ID NO. 374 | SEQ ID NO. 375 | SEQ ID NO. 376 |
| VH51 | SEQ ID NO. 377 | SEQ ID NO. 378 | SEQ ID NO. 379 |
| VH52 | SEQ ID NO. 389 | SEQ ID NO. 390 | SEQ ID NO. 391 |
| VH53 | SEQ ID NO. 392 | SEQ ID NO. 393 | SEQ ID NO. 394 |
| VH54 | SEQ ID NO. 395 | SEQ ID NO. 396 | SEQ ID NO. 397 |
| VH55 | SEQ ID NO. 407 | SEQ ID NO. 408 | SEQ ID NO. 409 |
| VH56 | SEQ ID NO. 410 | SEQ ID NO. 411 | SEQ ID NO. 412 |
| VH57 | SEQ ID NO. 413 | SEQ ID NO. 414 | SEQ ID NO. 415 |
| VH58 | SEQ ID NO. 425 | SEQ ID NO. 426 | SEQ ID NO. 427 |
| VH59 | SEQ ID NO. 428 | SEQ ID NO. 429 | SEQ ID NO. 430 |
| VH60 | SEQ ID NO. 431 | SEQ ID NO. 432 | SEQ ID NO. 433 |
| VH61 | SEQ ID NO. 443 | SEQ ID NO. 444 | SEQ ID NO. 445 |
| VH62 | SEQ ID NO. 446 | SEQ ID NO. 447 | SEQ ID NO. 448 |
| VH63 | SEQ ID NO. 449 | SEQ ID NO. 450 | SEQ ID NO. 451 |
| VH64 | SEQ ID NO. 461 | SEQ ID NO. 462 | SEQ ID NO. 463 |
| VH65 | SEQ ID NO. 464 | SEQ ID NO. 465 | SEQ ID NO. 466 |
| VH66 | SEQ ID NO. 467 | SEQ ID NO. 468 | SEQ ID NO. 469 |
| VH67 | SEQ ID NO. 479 | SEQ ID NO. 480 | SEQ ID NO. 481 |
| VH68 | SEQ ID NO. 482 | SEQ ID NO. 483 | SEQ ID NO. 484 |
| VH69 | SEQ ID NO. 485 | SEQ ID NO. 486 | SEQ ID NO. 487 |
| VH70 | SEQ ID NO. 497 | SEQ ID NO. 498 | SEQ ID NO. 499 |
| VH71 | SEQ ID NO. 500 | SEQ ID NO. 501 | SEQ ID NO. 502 |
| VH72 | SEQ ID NO. 503 | SEQ ID NO. 504 | SEQ ID NO. 505 |
| VH73 | SEQ ID NO. 515 | SEQ ID NO. 516 | SEQ ID NO. 517 |
| VH74 | SEQ ID NO. 518 | SEQ ID NO. 519 | SEQ ID NO. 520 |
| VH75 | SEQ ID NO. 521 | SEQ ID NO. 522 | SEQ ID NO. 523 |
| VH76 | SEQ ID NO. 533 | SEQ ID NO. 534 | SEQ ID NO. 535 |
| VH77 | SEQ ID NO. 536 | SEQ ID NO. 537 | SEQ ID NO. 538 |
| VH78 | SEQ ID NO. 539 | SEQ ID NO. 540 | SEQ ID NO. 541 |
| VH79 | SEQ ID NO. 551 | SEQ ID NO. 552 | SEQ ID NO. 553 |
| VH80 | SEQ ID NO. 554 | SEQ ID NO. 555 | SEQ ID NO. 556 |
| VH81 | SEQ ID NO. 557 | SEQ ID NO. 558 | SEQ ID NO. 559 |
| VH82 | SEQ ID NO. 569 | SEQ ID NO. 570 | SEQ ID NO. 571 |
| VH83 | SEQ ID NO. 572 | SEQ ID NO. 573 | SEQ ID NO. 574 |
| VH84 | SEQ ID NO. 575 | SEQ ID NO. 576 | SEQ ID NO. 577 |
| VH85 | SEQ ID NO. 587 | SEQ ID NO. 588 | SEQ ID NO. 589 |
| VH86 | SEQ ID NO. 590 | SEQ ID NO. 591 | SEQ ID NO. 592 |
| VH87 | SEQ ID NO. 593 | SEQ ID NO. 594 | SEQ ID NO. 595 |
| VH88 | SEQ ID NO. 605 | SEQ ID NO. 606 | SEQ ID NO. 607 |
| VH89 | SEQ ID NO. 608 | SEQ ID NO. 609 | SEQ ID NO. 610 |
| VH90 | SEQ ID NO. 611 | SEQ ID NO. 612 | SEQ ID NO. 613 |
| VH91 | SEQ ID NO. 623 | SEQ ID NO. 624 | SEQ ID NO. 625 |
| VH92 | SEQ ID NO. 626 | SEQ ID NO. 627 | SEQ ID NO. 628 |
| VH93 | SEQ ID NO. 629 | SEQ ID NO. 630 | SEQ ID NO. 631 |
| VH94 | SEQ ID NO. 641 | SEQ ID NO. 642 | SEQ ID NO. 643 |
| VH95 | SEQ ID NO. 644 | SEQ ID NO. 645 | SEQ ID NO. 646 |
| VH96 | SEQ ID NO. 647 | SEQ ID NO. 648 | SEQ ID NO. 649 |
| VH97 | SEQ ID NO. 659 | SEQ ID NO. 660 | SEQ ID NO. 661 |
| VH98 | SEQ ID NO. 662 | SEQ ID NO. 663 | SEQ ID NO. 664 |
| VH99 | SEQ ID NO. 665 | SEQ ID NO. 666 | SEQ ID NO. 667 |
| VH100 | SEQ ID NO. 677 | SEQ ID NO. 678 | SEQ ID NO. 679 |
| VH101 | SEQ ID NO. 680 | SEQ ID NO. 681 | SEQ ID NO. 682 |
| VH102 | SEQ ID NO. 683 | SEQ ID NO. 684 | SEQ ID NO. 685 |
| VH103 | SEQ ID NO. 695 | SEQ ID NO. 696 | SEQ ID NO. 697 |
| VH104 | SEQ ID NO. 698 | SEQ ID NO. 699 | SEQ ID NO. 700 |
| VH105 | SEQ ID NO. 701 | SEQ ID NO. 702 | SEQ ID NO. 703 |

and,
(2) the combination of light chain CDRs comprises: CDR1-VL, CDR2-VL, and CDR3-VL, the CDR1-VL, CDR2-VL and CDR3-VL have any sequence combination selected from the following, or a sequence combination with 1, 2, 3 or more amino acid insertions, deletions and/or substitutions compared to the sequence combination:

| | SEQ ID NO. | | |
|---|---|---|---|
| No. | CDR1-VL | CDR2-VL | CDR3-VL |
| VL1 | SEQ ID NO. 92 | SEQ ID NO. 93 | SEQ ID NO. 94 |
| VL2 | SEQ ID NO. 95 | SEQ ID NO. 96 | SEQ ID NO. 97 |
| VL3 | SEQ ID NO. 98 | SEO ID NO. 99 | SEQ ID NO. 100 |
| VL4 | SEQ ID NO. 110 | SEQ ID NO. 111 | SEQ ID NO. 112 |
| VL5 | SEQ ID NO. 113 | SEQ ID NO. 114 | SEQ ID NO. 115 |
| VL6 | SEQ ID NO. 116 | SEQ ID NO. 117 | SEQ ID NO. 118 |
| VL7 | SEQ ID NO. 128 | SEQ ID NO. 129 | SEQ ID NO. 130 |
| VL8 | SEQ ID NO. 131 | SEQ ID NO. 132 | SEQ ID NO. 133 |
| VL9 | SEQ ID NO. 134 | SEQ ID NO. 135 | SEQ ID NO. 136 |
| VL10 | SEQ ID NO. 146 | SEQ ID NO. 147 | SEQ ID NO. 148 |
| VL11 | SEQ ID NO. 149 | SEQ ID NO. 150 | SEQ ID NO. 151 |
| VL12 | SEQ ID NO. 152 | SEQ ID NO. 153 | SEQ ID NO. 154 |
| VL13 | SEQ ID NO. 164 | SEQ ID NO. 165 | SEQ ID NO. 166 |
| VL14 | SEQ ID NO. 167 | SEQ ID NO. 168 | SEQ ID NO. 169 |
| VL15 | SEQ ID NO. 170 | SEQ ID NO. 171 | SEQ ID NO. 172 |
| VL16 | SEQ ID NO. 182 | SEQ ID NO. 183 | SEQ ID NO. 184 |
| VL17 | SEQ ID NO. 185 | SEQ ID NO. 186 | SEQ ID NO. 187 |
| VL18 | SEQ ID NO. 188 | SEQ ID NO. 189 | SEQ ID NO. 190 |
| VL19 | SEQ ID NO. 200 | SEQ ID NO. 201 | SEQ ID NO. 202 |
| VL20 | SEQ ID NO. 203 | SEQ ID NO. 204 | SEQ ID NO. 205 |
| VL21 | SEQ ID NO. 206 | SEQ ID NO. 207 | SEQ ID NO. 208 |
| VL22 | SEQ ID NO. 218 | SEQ ID NO. 219 | SEQ ID NO. 220 |
| VL23 | SEQ ID NO. 221 | SEQ ID NO. 222 | SEQ ID NO. 223 |
| VL24 | SEQ ID NO. 224 | SEQ ID NO. 225 | SEQ ID NO. 226 |
| VL25 | SEQ ID NO. 236 | SEQ ID NO. 237 | SEQ ID NO. 238 |
| VL26 | SEQ ID NO. 239 | SEQ ID NO. 240 | SEQ ID NO. 241 |
| VL27 | SEQ ID NO. 242 | SEQ ID NO. 243 | SEQ ID NO. 244 |
| VL28 | SEQ ID NO. 254 | SEQ ID NO. 255 | SEQ ID NO. 256 |
| VL29 | SEQ ID NO. 257 | SEQ ID NO. 258 | SEQ ID NO. 259 |
| VL30 | SEQ ID NO. 260 | SEQ ID NO. 261 | SEQ ID NO. 262 |
| VL31 | SEQ ID NO. 272 | SEQ ID NO. 273 | SEQ ID NO. 274 |
| VL32 | SEQ ID NO. 275 | SEQ ID NO. 276 | SEQ ID NO. 277 |
| VL33 | SEQ ID NO. 278 | SEQ ID NO. 279 | SEQ ID NO. 280 |
| VL34 | SEQ ID NO. 290 | SEQ ID NO. 291 | SEQ ID NO. 292 |
| VL35 | SEQ ID NO. 293 | SEQ ID NO. 294 | SEQ ID NO. 295 |
| VL36 | SEQ ID NO. 296 | SEQ ID NO. 297 | SEQ ID NO. 298 |
| VL37 | SEQ ID NO. 308 | SEQ ID NO. 309 | SEQ ID NO. 310 |
| VL38 | SEQ ID NO. 311 | SEQ ID NO. 312 | SEQ ID NO. 313 |
| VL39 | SEQ ID NO. 314 | SEQ ID NO. 315 | SEQ ID NO. 316 |
| VL40 | SEQ ID NO. 326 | SEQ ID NO. 327 | SEQ ID NO. 328 |
| VL41 | SEQ ID NO. 329 | SEQ ID NO. 330 | SEQ ID NO. 331 |
| VL42 | SEQ ID NO. 332 | SEQ ID NO. 333 | SEQ ID NO. 334 |
| VL43 | SEQ ID NO. 344 | SEQ ID NO. 345 | SEQ ID NO. 346 |
| VL44 | SEQ ID NO. 347 | SEQ ID NO. 348 | SEQ ID NO. 349 |
| VL45 | SEQ ID NO. 350 | SEQ ID NO. 351 | SEQ ID NO. 352 |
| VL46 | SEQ ID NO. 362 | SEQ ID NO. 363 | SEQ ID NO. 364 |
| VL47 | SEQ ID NO. 365 | SEQ ID NO. 366 | SEQ ID NO. 367 |
| VL48 | SEQ ID NO. 368 | SEQ ID NO. 369 | SEQ ID NO. 370 |
| VL49 | SEQ ID NO. 380 | SEQ ID NO. 381 | SEQ ID NO. 382 |
| VL50 | SEQ ID NO. 383 | SEQ ID NO. 384 | SEQ ID NO. 385 |
| VL51 | SEQ ID NO. 386 | SEQ ID NO. 387 | SEQ ID NO. 388 |
| VL52 | SEQ ID NO. 398 | SEQ ID NO. 399 | SEQ ID NO. 400 |
| VL53 | SEQ ID NO. 401 | SEQ ID NO. 402 | SEQ ID NO. 403 |
| VL54 | SEQ ID NO. 404 | SEQ ID NO. 405 | SEQ ID NO. 406 |
| VL55 | SEQ ID NO. 416 | SEQ ID NO. 417 | SEQ ID NO. 418 |
| VL56 | SEQ ID NO. 419 | SEQ ID NO. 420 | SEQ ID NO. 421 |
| VL57 | SEQ ID NO. 422 | SEQ ID NO. 423 | SEQ ID NO. 424 |
| VL58 | SEQ ID NO. 434 | SEQ ID NO. 435 | SEQ ID NO. 436 |
| VL59 | SEQ ID NO. 437 | SEQ ID NO. 438 | SEQ ID NO. 439 |
| VL60 | SEQ ID NO. 440 | SEQ ID NO. 441 | SEQ ID NO. 442 |
| VL61 | SEQ ID NO. 452 | SEQ ID NO. 453 | SEQ ID NO. 454 |
| VL62 | SEQ ID NO. 455 | SEQ ID NO. 456 | SEQ ID NO. 457 |
| VL63 | SEQ ID NO. 458 | SEQ ID NO. 459 | SEQ ID NO. 460 |
| VL64 | SEQ ID NO. 470 | SEQ ID NO. 471 | SEQ ID NO. 472 |
| VL65 | SEQ ID NO. 473 | SEQ ID NO. 474 | SEQ ID NO. 475 |
| VL66 | SEQ ID NO. 476 | SEQ ID NO. 477 | SEQ ID NO. 478 |
| VL67 | SEQ ID NO. 488 | SEQ ID NO. 489 | SEQ ID NO. 490 |
| VL68 | SEQ ID NO. 491 | SEQ ID NO. 492 | SEQ ID NO. 493 |
| VL69 | SEQ ID NO. 494 | SEQ ID NO. 495 | SEQ ID NO. 496 |
| VL70 | SEQ ID NO. 506 | SEQ ID NO. 507 | SEQ ID NO. 508 |
| VL71 | SEQ ID NO. 509 | SEQ ID NO. 510 | SEQ ID NO. 511 |
| VL72 | SEQ ID NO. 512 | SEQ ID NO. 513 | SEQ ID NO. 514 |
| VL73 | SEQ ID NO. 524 | SEQ ID NO. 525 | SEQ ID NO. 526 |
| VL74 | SEQ ID NO. 527 | SEQ ID NO. 528 | SEQ ID NO. 529 |
| VL75 | SEQ ID NO. 530 | SEQ ID NO. 531 | SEQ ID NO. 532 |
| VL76 | SEQ ID NO. 542 | SEQ ID NO. 543 | SEQ ID NO. 544 |
| VL77 | SEQ ID NO. 545 | SEQ ID NO. 546 | SEQ ID NO. 547 |
| VL78 | SEQ ID NO. 548 | SEQ ID NO. 549 | SEQ ID NO. 550 |
| VL79 | SEQ ID NO. 560 | SEQ ID NO. 561 | SEQ ID NO. 562 |
| VL80 | SEQ ID NO. 563 | SEQ ID NO. 564 | SEQ ID NO. 565 |
| VL81 | SEQ ID NO. 566 | SEQ ID NO. 567 | SEQ ID NO. 568 |
| VL82 | SEQ ID NO. 578 | SEQ ID NO. 579 | SEQ ID NO. 580 |
| VL83 | SEQ ID NO. 581 | SEQ ID NO. 582 | SEQ ID NO. 583 |
| VL84 | SEQ ID NO. 584 | SEQ ID NO. 585 | SEQ ID NO. 586 |
| VL85 | SEQ ID NO. 596 | SEQ ID NO. 597 | SEQ ID NO. 598 |
| VL86 | SEQ ID NO. 599 | SEQ ID NO. 600 | SEQ ID NO. 601 |
| VL87 | SEQ ID NO. 602 | SEQ ID NO. 603 | SEQ ID NO. 604 |
| VL88 | SEQ ID NO. 614 | SEQ ID NO. 615 | SEQ ID NO. 616 |
| VL89 | SEQ ID NO. 617 | SEQ ID NO. 618 | SEQ ID NO. 619 |
| VL90 | SEQ ID NO. 620 | SEQ ID NO. 621 | SEQ ID NO. 622 |
| VL91 | SEQ ID NO. 632 | SEQ ID NO. 633 | SEQ ID NO. 634 |
| VL92 | SEQ ID NO. 635 | SEQ ID NO. 636 | SEQ ID NO. 637 |
| VL93 | SEQ ID NO. 638 | SEQ ID NO. 639 | SEQ ID NO. 640 |
| VL94 | SEQ ID NO. 650 | SEQ ID NO. 651 | SEQ ID NO. 652 |
| VL95 | SEQ ID NO. 653 | SEQ ID NO. 654 | SEQ ID NO. 655 |
| VL96 | SEQ ID NO. 656 | SEQ ID NO. 657 | SEQ ID NO. 658 |
| VL97 | SEQ ID NO. 668 | SEQ ID NO. 669 | SEQ ID NO. 670 |
| VL98 | SEQ ID NO. 671 | SEQ ID NO. 672 | SEQ ID NO. 673 |
| VL99 | SEQ ID NO. 674 | SEQ ID NO. 675 | SEQ ID NO. 676 |
| VL100 | SEQ ID NO. 686 | SEQ ID NO. 687 | SEQ ID NO. 688 |
| VL101 | SEQ ID NO. 689 | SEQ ID NO. 690 | SEQ ID NO. 691 |
| VL102 | SEQ ID NO. 692 | SEQ ID NO. 693 | SEQ ID NO. 694 |
| VL103 | SEQ ID NO. 704 | SEQ ID NO. 705 | SEQ ID NO. 706 |
| VL104 | SEQ ID NO. 707 | SEQ ID NO. 708 | SEQ ID NO. 709 |
| VL105 | SEQ ID NO. 710 | SEQ ID NO. 711 | SEQ ID NO. 712 |

each CDR1-VH, CDR2-VH, CDR3-VH, CDR1-VL, CDR2-VL and CDR3-VL is coded according to the prevailing analysis methods of KABAT, Chothia or IMGT.

In particular, for example, the antibody or the antigen-binding fragment thereof of the invention comprises a combination of a heavy chain CDR and a light chain CDR selected from: VH1+VL1, VH2+VL2, VH3+VL3, VH4+VL4, VH5+VL5, VH6+VL6, VH7+VL7, VH8+VL8, VH9+VL9, VH10+VL10, VH11+VL11, VH12+VL12, VH13+VL13, VH14+VL14, VH15+VL15, VH16+VL16, VH17+VL17, VH18+VL18, VH19+VL19, VH20+VL20, VH21+VL21, VH22+VL22, VH23+VL23, VH24+VL24, VH25+VL25, VH26+VL26, VH27+VL27, VH28+VL28, VH29+VL29,

VH30+VL30, VH31+VL31, VH32+VL32, VH33+VL33, VH34+VL34, VH35+VL35, VH36+VL36, VH37+VL37, VH38+VL38, VH39+VL39, VH40+VL40, VH41+VL41, VH42+VL42, VH43+VL43, VH44+VL44, VH45+VL45, VH46+VL46, VH47+VL47, VH48+VL48, VH49+VL49, VH50+VL50, VH51+VL51, VH52+VL52, VH53+VL53, VH54+VL54, VH55+VL55, VH56+VL56, VH57+VL57, VH58+VL58, VH59+VL59, VH60+VL60, VH61+VL61, VH62+VL62, VH63+VL63, VH64+VL64, VH65+VL65, VH66+VL66, VH67+VL67, VH68+VL68, VH69+VL69, VH70+VL70, VH71+VL71, VH72+VL72, VH73+VL73, VH74+VL74, VH75+VL75, VH76+VL76, VH77+VL77, VH78+VL78, VH79+VL79, VH80+VL80, VH81+VL81, VH82+VL82, VH83+VL83, VH84+VL84, VH85+VL85, VH86+VL86, VH87+VL87, VH88+VL88, VH89+VL89, VH90+VL90, VH91+VL91, VH92+VL92, VH93+VL93, VH94+VU4, VH95+VL95, VH96+VL96, VH97+VL97, VH98+VL98, VH99+VL99, VH100+VL100, VH101+VL101, VH102+VL102, VH103+VL103, VH104+VL104, or VH105+VL105, and a combination of CDRs with insertions, deletions and/or substitutions of 1, 2, 3 or more amino acids compared to the sequence of the heavy chain CDR and the light chain CDR in these combinations.

In a specific embodiment, the invention provides an antibody or an antigen-binding fragment thereof comprising:
(1) a heavy chain variable region having a sequence as shown in SEQ ID NO: 13,15,17,19,21,23,25,27,29,31,33,35,37,39,41,43,45,47,49,51,53,55,57, 59, 61, 63, 65, 67, 69, 71, 73, 75, 77, 79, or 81; a light chain variable region having a sequence as shown in SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, or 82;
(2) an amino acid sequence having at least 90% identity, preferably at least 91%, 92%, 93%, 94%, 95%, 96%, 97%, 98% or 99% identity with the sequence shown in (1) above; or,
(3) a framework region of the antibody or the antigen-binding fragment having at least 90% identity, preferably at least 91%, 92%, 93%, 94%, 95%, 96%, 97%, 98%, 99% identity with the framework region of the amino acid sequence as shown in (1) above.

In a preferred embodiment, the dissociation constant (KD) of the antibody or the antigen-binding fragment of the present invention binding to human CD22 is no more than 10⁻⁶ M, and the dissociation constant (KD) of the antibody or the antigen-binding fragment of the present invention binding to rhesus monkey CD22 (KD) is no more than 10⁻⁸ M.

Or, optionally, the antibody or the antigen-binding fragment binds or does not bind to monkey CD22;

Optionally, the antibody or the antigen-binding fragment binds to or does not bind to murine CD22.

In a preferred embodiment, the antibody or the antigen-binding fragment of the invention is chimeric or humanized or fully human; preferably, the antibody or the antigen-binding fragment is selected from a monoclonal antibody, a polyclonal antibody, a natural antibody, an engineered antibody, a monospecific antibody, a multispecific antibody (for example, a bispecific antibody), a monovalent antibody, a multivalent antibody, a full-length antibody, an antibody fragment, a naked antibody, a conjugated antibody, a humanized antibody, a fully human antibody, Fab, Fab′, F(ab′)2, Fd, Fv, scFv, a diabody or a single domain antibody.

In a preferred embodiment, the antibody or the antigen-binding fragment thereof of the present invention comprises a sequence of the constant region of any one of human or murine antibody IgG1, IgG2, IgG3, IgG4, IgA, IgM, IgE or IgD; preferably, comprises a sequence of the constant region of human or murine antibody IgG1, IgG2, IgG3 or IgG4; or a sequence of the constant region of human or murine antibody IgG1, IgG2, IgG3 or IgG4 carrying a mutation.

In a preferred embodiment, the antigen-binding fragment of the present invention is selected from one or more of F(ab)2, Fab', Fab, Fv, scFv bispecific antibody, a nanobody and an antibody minimum recognition unit.

In a preferred embodiment, the antibody or the antigen-binding fragment thereof of the invention is further coupled with a therapeutic agent or a tracer; preferably, the therapeutic agent is selected from a radioisotope, a chemotherapeutic agent or an immunomodulator, and the tracer is selected from a radiological contrast agent, a paramagnetic ion, a metal, a fluorescent label, a chemiluminescence label, a ultrasound contrast agent or a photosensitizer.

In a preferred embodiment, the present invention also provides a multispecific antigen-binding molecule; preferably, the multispecific antigen-binding molecule comprises a first antigen-binding module and a second antigen-binding module, the first antigen-binding module comprises the antibody or the antigen-binding fragment described in any one of the above, the second antigen-binding module specifically binds to other antigens than CD22 or binds to a different CD22 epitope than the first antigen-binding module;
preferably, the other antigens are selected from CD3, CD16, CD16A, CD4, CDS, CD8, CD14, CD15, CD19, CD20, CD21, CD23, CD25, CD33, CD37, CD38, CD40, CD40L, CD46, CD52, CD54 , CD66(a-d), CD74, CD80, CD126, CD138, B7, MUC, Ia, HLA-DR, tenascin, VEGF, P1GF, ED-B fibronectin, oncogene products, IL-2, IL-6, TRAIL-R1 or TRAIL-R2;
preferably, the multispecific antibody is a bispecific antibody, a trispecific antibody or a tetraspecific antibody.

In a preferred embodiment, the present invention provides a chimeric antigen receptor (CAR); preferably, the chimeric antigen receptor at least comprises an extracellular antigen-binding domain, a transmembrane domain and an intracellular signaling domain, and the extracellular antigen-binding domain comprises any of the CD22 antibody or the antigen-binding fragment described above.

In a preferred embodiment, the present invention provides an immune effector cell; preferably, the immune effector cell comprises the chimeric antigen receptor described above or a nucleic acid fragment encoding the chimeric antigen receptor described above;
preferably, the immune effector cell is selected from a T cell, a NK cell (a natural killer cell), a NKT cell (a natural killer T cell), a monocyte, a macrophage, a dendritic cell or a mast cell; the T cell may be selected from an inflammatory T cell, a cytotoxic T cell, a regulatory T cell (Treg) or a helper T cell;
preferably, the immune effector cell is an allogeneic immune effector cell or an autologous immune cell.

In a preferred embodiment, the present invention provides an isolated nucleic acid molecule encoding the nanobody, the antigen-binding fragment, or any combination thereof according to any one of the above aspects of the present invention, the multispecific antigen-binding molecule described above or the chimeric antigen receptor described above.

In some embodiments, the present invention provides an expression vector comprising the isolated nucleic acid molecule of the present invention described above.

In some embodiments, the present invention provides a host cell comprising the isolated nucleic acid molecule or the expression vector of the present invention described above.

In a preferred embodiment, the host cell is a eukaryotic cell or a prokaryotic cell; more preferably, the host cell is derived from a mammalian cell, a yeast cell, an insect cell, Escherichia coli and/or Bacillus subtilis; more preferably, the host cell is selected from HEK293E or Chinese hamster ovary (CHO) cell.

In some embodiments, the present invention provides a method for preparing an antibody or an antigen-binding fragment or a multispecific antigen-binding molecule, the method comprises culturing or culturing, under appropriate conditions, the host cell of the present invention described above, and isolating the antibody or the antigen-binding fragment or the multispecific antigen-binding molecule.

In some embodiments, the present invention provides a method for preparing an immune effector cell, wherein the CAR nucleic acid fragment described above is introduced into the immune effector cell, preferably, the method further comprises enabling the immune effector cell to express the CAR described above.

In some embodiments, the present invention provides a pharmaceutical composition comprising the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, or a product (e.g., an antibody and an antigen-binding fragment) prepared by the method of the invention described above, and a pharmaceutically acceptable carrier.

In a preferred embodiment, the pharmaceutical composition further comprises a pharmaceutically acceptable carrier, diluent or adjuvant; more preferably, the pharmaceutical composition further comprises an additional antineoplastic agent.

In some embodiments, the present invention provides a method for preventing and/or treating a B cell disease, the method comprises administering the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above to a patient in need thereof. The B cell disease is preferably a tumor or an autoimmune disease;

preferably, the tumor is selected from lymphoma or leukemia, the lymphoma or leukemia is selected from B-cell lymphoma, non-Hodgkin's lymphoma, mantle cell lymphoma, follicular lymphoma, marginal zone lymphoma, primary mediastinal B-cell lymphoma, diffuse large B-cell lymphoma, precursor B-cell acute lymphocytic leukemia (pre-B ALL), acute lymphocytic leukemia (ALL), chronic lymphocytic leukemia, multiple myeloma;

preferably, the autoimmune disease is selected from systemic lupus erythematosus (SLE), antiphospholipid antibody syndrome, multiple sclerosis, ulcerative colitis, Crohn's disease, rheumatoid arthritis, Sjogren's syndrome, Guillain-Barre syndrome, myasthenia gravis, large vessel vasculitis, medium vessel vasculitis, polyarteritis nodosa, pemphigus, scleroderma, pulmonary hemorrhage-nephritic syndrome, glomerular nephritis, primary biliary cirrhosis, Graves' disease, membranous nephropathy, autoimmune hepatitis, sprue, Addison's disease, polymyositis/dermatomyositis, monoclonal gamma globulin disease, factor VIII deficiency, cryoglobulinemia, peripheral neuropathy, IgM polyneuropathy, chronic neuropathy, and chronic lymphocytic thyroiditis.

In some embodiments, the present invention provides the use of the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above in the preparation of a drug for preventing and/or treating a B cell disease, and the B cell disease is preferably a tumor or an autoimmune disease;
preferably, the tumor is selected from lymphoma or leukemia, the lymphoma or leukemia is selected from B-cell lymphoma, non-Hodgkin's lymphoma, mantle cell lymphoma, follicular lymphoma, marginal zone lymphoma, primary mediastinal B-cell lymphoma, diffuse large B-cell lymphoma, precursor B-cell acute lymphocytic leukemia (pre-B ALL), acute lymphocytic leukemia (ALL), chronic lymphocytic leukemia, multiple myeloma;
preferably, the autoimmune disease is selected from systemic lupus erythematosus (SLE), antiphospholipid antibody syndrome, multiple sclerosis, ulcerative colitis, Crohn's disease, rheumatoid arthritis, Sjogren's syndrome, Guillain-Barre syndrome, myasthenia gravis, large vessel vasculitis, medium vessel vasculitis, polyarteritis nodosa, pemphigus, scleroderma, pulmonary hemorrhage-nephritic syndrome, glomerular nephritis, primary biliary cirrhosis, Graves' disease, membranous nephropathy, autoimmune hepatitis, sprue, Addison's disease, polymyositis/dermatomyositis, monoclonal gamma globulin disease, factor VIII deficiency, cryoglobulinemia, peripheral neuropathy, IgM polyneuropathy, chronic neuropathy, and chronic lymphocytic thyroiditis.

In some embodiments, the present invention provides the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above for preventing and/or treating a B cell disease; The B cell disease is preferably a tumor or an autoimmune disease;
preferably, the tumor is selected from lymphoma or leukemia, the lymphoma or leukemia is selected from B-cell lymphoma, non-Hodgkin's lymphoma, mantle cell lymphoma, follicular lymphoma, marginal zone lymphoma, primary mediastinal B-cell lymphoma, diffuse large B-cell lymphoma, precursor B-cell acute lymphocytic leukemia (pre-B ALL), acute lymphocytic leukemia (ALL), chronic lymphocytic leukemia, multiple myeloma;
preferably, the autoimmune disease is selected from systemic lupus erythematosus (SLE), antiphospholipid antibody syndrome, multiple sclerosis, ulcerative colitis, Crohn's disease, rheumatoid arthritis, Sjogren's syndrome, Guillain-Barre syndrome, myasthenia gravis, large vessel vasculitis, medium vessel vasculitis, polyarteritis nodosa, pemphigus, scleroderma, pulmonary hemorrhage-nephritic syndrome, glomerular nephritis, primary biliary cirrhosis, Graves' disease, membranous nephropathy, autoimmune hepatitis, sprue, Addison's disease, polymyositis/dermatomyositis, monoclonal gamma globulin disease, factor VIII deficiency, cryoglobulinemia, peripheral neuropathy, IgM polyneuropathy, chronic neuropathy, and chronic lymphocytic thyroiditis.

In some embodiments, the present invention provides a kit comprising the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, or a product (e.g., an antibody and an antigen-binding fragment) prepared by the method of the invention described above, or the pharmaceutical composition of the invention described above, and instructions for use.

### Definition and Description of Terminology

As used herein, the term “antibody” (Ab) refers to an immunoglobulin molecule that specifically binds to or is immunoreactive with an antigen of interest and includes polyclonal, monoclonal, genetically engineered, and other modified forms of an antibody (including but not limited to a chimeric antibody, a humanized antibody, a fully human antibody, a heteroconjugate antibody (e.g. a bispecific, trispecific and tetraspecific antibody, a diabody, a triabody and a tetrabody, an antibody conjugate)) and an antigen-binding fragment of an antibody (including, for example, Fab′, F(ab′)2, Fab, Fv, rIgG and scFv fragment). Furthermore, unless otherwise indicated, the term “monoclonal antibody” (mAb) is intended to include both an intact antibody molecule and an incomplete antibody fragment (such as Fab and F(ab′)2 fragment, which lacks the Fc fragment of the intact antibody (cleared more quickly from circulation in animals), thus lacking Fc-mediated effector function) capable of specifically binding to a target protein (see Wahl et al., J. Nucl. Med. 24: 316, 1983; the content of which is incorporated herein by reference).

An “antibody” herein may be derived from any animal, including but not limited to humans and non-human animals selected from primates, mammals, rodents and vertebrates, such as camelids, llamas, guanaco, alpaca, sheep, rabbits, mice, rats or cartilaginous fishes (such as sharks).

The term “natural antibody” herein refers to an antibody produced and paired by the immune system of a multicellular organism. The term “engineered antibody” herein refers to a non-natural antibody obtained through genetic engineering, antibody engineering, etc. For example, “engineered antibody” includes a humanized antibody, a small molecule antibody (such as scFv, etc.), a bispecific antibody, etc.

The term “monospecific” herein refers to having one or more binding sites, wherein each binding site binds the same epitope of the same antigen.

The term “multispecific” herein refers to having at least two antigen binding sites, each of which binds a different epitope of the same antigen or a different epitope of a different antigen. Thus, terms such as “bispecific”, “trispecific”, “tetraspecific” and the like refer to the number of different epitopes to which an antibody/an antigen-binding molecule can bind.

The term “valence” herein refers to the presence of a defined number of binding sites in an antibody/an antigen-binding molecule. Thus, the terms “monovalent”, “bivalent”, “tetravalent” and “hexavalent” refer to the presence of one binding site, two binding sites, four binding site and six binding sites in an antibody/an antigen-binding molecule, respectively.

“Full-length antibody”, “complete antibody” and “intact antibody” are used interchangeably herein to mean that they have a structure substantially similar to that of a natural antibody.

As used herein, the term “antigen-binding fragment” refers to one or more antibody fragments that retain the ability to specifically bind to a target antigen. The antigen-binding function of an antibody can be performed by a fragment of a full-length antibody. The antibody fragment can be Fab, F(ab′)2, scFv, SMIP, a diabody, a triabody, an affibody, a nanobody, an aptamer or a domain antibody. Examples of a binding fragment encompassed by the term “antigen-binding fragment” of an antibody include, but are not limited to: (i) an Fab fragment, which is a monovalent fragment consisting of VL, VH, CL and CH1 domains; (ii) an F(ab)2 fragment, which is a bivalent fragment comprising two Fab fragments connected by a disulfide bond in the hinge region; (iii) an Fd fragment consisting of VH and CH1 domains; (iv) an Fv fragment consisting of the VL and VH domains of a single arm of an antibody; (V) dAb comprising VH and VL domains; (vi) dAb fragment consisting of a VH domain (Ward et al., Nature 341: 544-546, 1989); (vii) dAb consisting of VH or VL domains; (viii) an isolated complementarity determining region (CDR); and (ix) a combination of two or more isolated CDRs, the CDRs may optionally be linked by a synthetic linker.

Furthermore, although the two domains VL and VH of the Fv fragment are encoded by separate genes, these two domains can be joined using a recombinant method through a linker that enables forming a single protein chain (referred to as a single chain Fv (scFv); see e.g., Bird et al., Science 242: 423-426, 1988 and Huston et al., Proc. Natl. Acad. Sci. USA 85: 5879-5883, 1988) in which the VL and VH regions pair to form a monovalent molecule. These antibody fragments can be obtained using conventional techniques known to those skilled in the art, and these fragments are screened for use in the same manner as an intact antibody. The antigen-binding fragment can be produced by recombinant DNA techniques, enzymatic or chemical cleavage of an intact immunoglobulin, or in some embodiments by chemical peptide synthesis procedures known in the art.

As used herein, the term “CD22” refers to Siglec-2, a molecule belonging to the SIGLEC lectin family, which is present on the surface of mature B cells and to a lesser extent on certain immature B cells. The term “CD22” includes CD22 proteins of any human and non-human animal species, and specifically includes human CD22 as well as CD22 of non-human mammals.

As used herein, the term “bispecific antibody” refers to an antibody, typically a human or humanized antibody, that has monoclonal binding specificities for at least two different antigens. In the present invention, one of the binding specificities can be detected against an antigen epitope of CD22, and the other can be detected against another antigen epitope of CD22 or any other antigen except CD22, such as a cell surface protein, a receptor, a receptor subunit, a tissue-specific antigen, a virus-derived protein, a virus-encoded envelope protein, a bacterium-derived protein, or a bacterial surface protein.

As used herein, the term “chimeric” antibody refers to an antibody that has a variable sequence derived from an immunoglobulin of one organism (such as a rat or mouse) and a constant region derived from an immunoglobulin of a different organism, such as human. Methods for producing the chimeric antibody are known in the art. See, e.g., Morrison, 1985, Science 229(4719): 1202-7; Oi et al., 1986, Bio Techniques 4: 214-221; Gillies et al., 1985 J Immunol Methods 125: 191-202; The above documents are incorporated herein by reference.

As used herein, the term “heavy chain antibody” refers to an antibody that lacks the light chains of an conventional antibody. The term specifically includes, but is not limited to, a homodimeric antibody comprising a VH antigen binding domain and CH2 and CH3 constant domains in the absence of a CH1 domain.

As used herein, the term “nanobody” refers to a natural heavy chain antibody without light chains in camel and cloning its variable region can obtain a single domain antibody, also known as VHH (Variable domain of heavy chain of heavy chain antibody), which only consists of a heavy chain variable region, and is the smallest functional antigen-binding fragment. For a further description of VHH and nanobody, reference is made to the review article by Muyldermans (2001, Reviews in Molecular Biotechnology 74:277-302), and to the following patent applications mentioned as general background art: WO 94/04678, WO 95/04079 and WO 96/34103 of the Free University of Brussels; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of Vlaams Instituut voor Biotechnologie (VIB); WO 03/050531 of

Algonomics N.V. and Ablynx N.V.; WO 01/90190 of the National Research Council of Canada; WO 03/025020 (=EP 1433793) of the Institute of Antibodies; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825 of Ablynx N.V., and further published patent applications of Ablynx N.V.

Reference is also made to the additional prior art mentioned in these applications, in particular the list of references mentioned on pages 41-43 of International Application WO 06/040153, which list and references are incorporated herein by reference. As described in these references, nanobody (in particular VHH sequences and partially humanized nanobody) may inter alia be characterized by the presence of one or more “signature residues” in one or more framework sequences. Further descriptions of nanobody can be found in, for example, WO 08/101985 and WO 08/142164, including humanization and/or camelization of nanobody, as well as other modifications, parts or fragments, derivatives or “nanobody fusion”, multivalent constructs (including some non-limiting examples of linker sequences) and various modifications that increase the half-life of a nanobody and a formulation thereof. For a further general description of nanobody , reference is made to the prior art cited herein, for example WO 08/020079 (page 16).

As used herein, the term “complementarity determining region” (CDR) refers to hypervariable regions found in both light and heavy chain variable domains. The more conserved portions in variable domains are called the framework regions (FR). As understood in the art, the amino acid positions representing the hypervariable regions of an antibody can vary according to the context and various definitions known in the art. Some positions within variable domains can be considered heterozygous hypervariable positions because these positions can be considered to be within the hypervariable regions under one set of criteria (such as IMGT or KABAT) but outside the hypervariable regions under a different set of criteria (such as KABAT or IMGT).

One or more of these positions may also be found in extended hypervariable regions. The invention includes an antibody comprising modifications in these hybrid hypervariable positions. The variable domains of the native heavy and light chains respectively comprise four framework regions that largely adopt a sheet configuration and connected by three CDRs (CDR1, CDR2, and CDR3) that form loops connecting the sheets, and in some cases form part of the sheet structure. The CDRs in each chain are held tightly together by the FR regions in the order of FR1-CDR1-FR2-CDR2-FR3-CDR3-FR4, and together with CDRs from other antibody chains contribute to the formation of the antibody's antigen-binding site (see Kabat et al., Sequences of Proteins of Immunological Interest, National Institute of Health, Bethesda, Md. 1987; which is incorporated herein by reference). For example, herein, CDR1-VH, CDR2-VH, and CDR3-VH refer to the first CDR, the second CDR, and the third CDR of the heavy chain variable region (VH), respectively, and these three CDRs constitute the CDR combination (VHCDR combination) of the heavy chain (or its variable region); CDR1-VL, CDR2-VL, and CDR3-VL refer to the first CDR, the second CDR, and the third CDR of the light chain variable region (VL), respectively, and these three CDRs constitute the CDR combination (VLCDR combination) of the light chain (or its variable region).

As used herein, the term “monoclonal antibody” refers to an antibody derived from a single clone (including any eukaryotic, prokaryotic, or phage clone), without limitation by the method by which the antibody is produced.

As used herein, the term “VH” refers to the variable region of an immunoglobulin heavy chain of an antibody, including the heavy chain of an Fv, scFv or Fab. The term “VL” refers to the variable region of an immunoglobulin light chain, including the light chain of an Fv, scFv, dsFv or Fab.

The term “heavy chain constant region” herein refers to the carboxy-terminal portion of the heavy chain of an antibody, which is not directly involved in the binding of the antibody to an antigen, but exhibits effector functions, such as interaction with Fc receptors, and has a more conserved amino acid sequence relative to the antibody's variable domain. A “heavy chain constant region” comprises at least one of the following: a CH1 domain, a hinge region, a CH2 domain, a CH3 domain, or a variant or fragment thereof “Heavy chain constant region” includes “full-length heavy chain constant region” and “heavy chain constant region fragment”, the former has a structure substantially similar to that of a natural antibody constant region, while the latter only includes “a part of the full-length heavy chain constant region”. For example, a typical “full-length antibody heavy chain constant region” consists of CH1 domain-hinge region-CH2 domain-CH3 domain; when the antibody is IgE, it also includes a CH4 domain; when the antibody is a heavy chain antibody, it does not include the CH1 domain. For example, a typical “heavy chain constant region fragment” can be selected from CH1, Fc or CH3 domains.

The term “light chain constant region” herein refers to the carboxy-terminal portion of the light chain of an antibody, which is not directly involved in the binding of the antibody to an antigen. The light chain constant region may be selected from a constant η domain or a constant λ domain.

The term “Fc” herein refers to the carboxy-terminal portion of an antibody obtained by papain hydrolysis of the intact antibody, which typically includes the CH3 and CH2 domains of the antibody. Fc region includes, for example, a native sequence Fc region, a recombinant Fc region and a variant Fc region. Although the boundary of the Fc region of an immunoglobulin heavy chain can vary slightly, the Fc region of a human IgG heavy chain is generally defined as extending from the amino acid residue at position Cys226 or from Pro230 to the carboxyl terminus. The C-terminal lysine of the Fc region (residue 447 according to the EU numbering system) may be removed, for example, during the production or purification of the antibody, or by recombinant engineering of the nucleic acid encoding the heavy chain of the antibody, thus the Fc region may comprise or may not comprise Lys447.

The term “humanized antibody” herein refers to a genetically engineered non-human antibody whose amino acid sequence has been modified to increase sequence homology with a human antibody. Generally speaking, all or part of the CDR regions of a humanized antibody are derived from a non-human antibody (donor antibody), and all or part of the non-CDR regions (for example, variable region FR and/or constant region) are derived from human Immunoglobulin (recipient antibody). Humanized antibody usually retains or partially retain the expected properties of the donor antibody, including but not limited to, antigen specificity, affinity, reactivity, ability to enhance immune cell activity, ability to enhance immune response, etc.

The term “fully human antibody” herein refers to an antibody having variable regions in which both the FRs and CDRs are derived from human germline immunoglobulin sequences. Furthermore, if the antibody comprises a constant region, the constant region also is derived from human germline immunoglobulin sequences. Fully human antibody herein may include amino acid residues not encoded by human germline immunoglobulin sequences (for example, mutations introduced by random or site-specific mutagenesis in vitro or by somatic mutation in vivo). However, a “fully human antibody” herein is not intended to include an antibody in which CDR sequences derived from the germline of another mammalian species (for example, mouse) have been grafted onto human framework sequences.

The term “naked antibody” herein refers to an antibody that is not linked, fused or conjugated to another agent or molecule (for example, label or drug), peptide or polypeptide. In specific embodiments, the naked antibody expressed by a mammalian host cell can be glycosylated by the host cell's glycosylation machinery (for example, glycosylase). In certain embodiments, the naked antibody is not glycosylated when expressed by a host cell that does not have its own glycosylation machinery (for example, glycosylase). In certain embodiments, the naked antibody is an intact antibody, while in other embodiments, the naked antibody is the antigen-binding fragment of an intact antibody, such as Fab antibody.

The term “conjugated antibody” herein refers to an antibody that can be associated with a pharmaceutically acceptable carrier or diluent and can be a monoclonal antibody, a chimeric antibody, a humanized antibody, or a human antibody.

The term “diabody” herein refers to bivalent bispecific antibody that can bind to different epitopes on the same or different antigens.

As used herein, the term “percent (%) sequence identity” refers to the percentage of amino acid (or nucleotide) residues of the candidate sequence that are identical to those of the reference sequence after aligning the sequences and introducing gaps (if necessary) in order to achieve maximum percentage sequence identity (for example, for optimal alignment, gaps can be introduced in one or both of the candidate sequence and the reference sequence, and non-homologous sequences can be ignored for comparison purpose). For purpose of determining percent sequence identity, alignment can be achieved in a variety of ways well known to those skilled in the art, for example, using publicly available computer software such as BLAST, ALIGN or Megalign (DNASTAIi) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence shows sequence identity from 50% to 100% in the full length of the candidate sequence or in the selected part of the continuous amino acid (or nucleotide) residues of the candidate sequence. The length of the candidate sequence aligned for comparison purpose is at least 30% (e.g., 30%, 40%, 50%, 60%, 70%, 80%, 90% or 100%) of the length of the reference sequence. When a position in the candidate sequence is occupied by the same amino acid (or nucleotide) residue as the corresponding position in the reference sequence, then the molecules are identical at that position.

The term “conservative amino acid” herein generally refers to amino acids that belong to the same class or have similar characteristics (for example, charge, side chain size, hydrophobicity, hydrophilicity, backbone conformation, and rigidity). For example, the amino acids in each of the following groups belong to each other's conservative amino acid residues, and the substitution of amino acid residues in the group belongs to the conservative amino acid substitution:
(1) acidic amino acids: Asp (D) and Glu (E);
(2) basic amino acids: Lys (K), Arg (R) and His (H):
(3) hydrophilic uncharged amino acids: Ser (S), Thr (T), Asn (N) and Gin (Q);
(4) Aliphatic uncharged amino acids: Gly (G), Ala (A), Val (V), Leu (L) and Ile (I);
(5) nonpolar uncharged amino acids: Cys (C), Met (M) and Pro (P);
(6) aromatic amino acids: Phe (F), Tyr (Y) and Trp (W).

The term “Kabat numbering system” herein generally refers to the immunoglobulin alignment and numbering system proposed by Elvin A. Kabat (see, for example, Kabat et al., Sequences of Proteins of Immunological Interest, 5th Ed. Public Health Service, National Institutes of Health, Bethesda, Md., 1991).

The term “Chothia numbering system” herein generally refers to the immunoglobulin numbering system proposed by Chothia et al., which is a classical rule for identifying the boundaries of CDR regions based on the location of structural loop regions (see, for example, Chothia & Lesk (1987) J. Mol . Biol. 196: 901-917; Chothia et al. (1989) Nature 342: 878-883).

The term “IMGT numbering system” herein generally refers to the immunoglobulin numbering system proposed by Chothia et al., which is a classical rule for identifying the boundaries of CDR regions based on the location of structural loop regions (see, for example, Chothia & Lesk (1987) J. Mol . Biol. 196: 901-917; Chothia et al. (1989) Nature 342: 878-883).

As used herein, the term “specific binding” refers to a binding reaction that determines the presence of an antigen in a heterogeneous population of proteins and other biomolecules, the proteins and other biomolecules are for example specifically recognized by an antibody or an antigen-binding fragment thereof. An antibody or an antigen-binding fragment thereof that specifically binds to an antigen would bind to the antigen with a KD of less than 100 nM. For example, an antibody or an antigen-binding fragment thereof that specifically binds to an antigen would bind to the antigen with a KD of up to 100 nM (for example, between 1 pM and 100 nM). An antibody or an antigen-binding fragment thereof that does not exhibit specific binding to a particular antigen or an epitope thereof would exhibit a KD for the particular antigen or the epitope thereof of greater than 100 nM (for example, greater than 500 nM, 1 μM, 100 μM, 500 μM, or 1 mM). Various immunoassays are available to select for an antibody that reacts specifically with a specific protein or carbohydrate. For example, solid-phase ELISA immunoassay is routinely used to select for an antibody that reacts specifically with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), which describes immunoassay methods and conditions that can be used to determine specific immunoreactivity.

As used herein, the term “antibody conjugate” refers to a coupled entity/conjugate formed by chemically bonding an antibody molecule to another molecule either directly or through a linker. Examples include antibody-drug conjugate (ADC), in which the drug molecule is said another molecule.

The term “chimeric antigen receptor (CAR)” herein refers to a recombinant protein comprising at least (1) an extracellular antigen-binding domain, such as a variable heavy or light chain of an antibody, and (2) a transmembrane domain used to make the anchored CAR enter immune effector cells, and (3) an intracellular signaling domain. In certain embodiments, the extracellular antigen binding domain of the CAR comprises a scFv. The scFv can be derived from the variable heavy and light regions of a fusion antibody. Alternatively or additionally, the scFv may be derived from Fab's (rather than an antibody, for example obtained from a Fab library). In certain embodiments, the scFv is fused to the transmembrane domain and then to the intracellular signaling domain.

Herein the term “nucleic acid” includes any compound and/or substance comprising a polymer of nucleotides. Each nucleotide consists of a base, specifically a purine or pyrimidine base (i.e., cytosine (C), guanine (G), adenine (A), thymine (T) or uracil (U)), a sugar (i.e., deoxyribose or ribose) and a phosphate group. Typically, the nucleic acid molecule is described by a sequence of bases, whereby the bases represent the primary structure (linear structure) of the nucleic acid molecule. The sequence of bases is usually expressed as 5′ to 3′. In this context, the term nucleic acid molecule encompasses deoxyribonucleic acid (DNA), including for example complementary DNA (cDNA) and genomic DNA, ribonucleic acid (RNA), especially messenger RNA (mRNA), synthetic forms of DNA or RNA, and a polymer containing a mixture of two or more of these molecules. The nucleic acid molecule can be linear or circular. Furthermore, the term nucleic acid molecule includes both sense strand and antisense strand, as well as single-stranded form and double-stranded form. Furthermore, the nucleic acid molecules described herein may contain naturally occurring or non-naturally occurring nucleotides. Examples of non-naturally occurring nucleotides include modified nucleotide bases with derivatized sugar or phosphate backbone linkages or chemically modified residues. The nucleic acid molecule also encompasses DNA and RNA molecules which are suitable as vectors for direct expression of the antibody of the invention in vitro and/or in vivo, for example in a host or patient. Such DNA (for example cDNA) or RNA (for example mRNA) vectors may be unmodified or modified. For example, mRNA can be chemically modified to enhance the stability of the RNA vector and/or the expression of the encoded molecule, so that the mRNA can be injected into a subject to generate an antibody in vivo (see, for example, Stadler et al., Nature Medicine 2017, Published online Jun. 12, 2017, doi: 10.1038/nm.4356 or EP 2 101 823 B1).

As used herein, the term “vector” includes a nucleic acid vector, such as a DNA vector (such as a plasmid), a RNA vector, a virus or other suitable replicons (such as viral vectors). A variety of vectors have been developed for the delivery of polynucleotides encoding foreign proteins into prokaryotic or eukaryotic cells. The expression vector of the invention contains polynucleotide sequences together with additional sequence elements, for example, for expressing proteins and/or integrating these polynucleotide sequences into the genome of mammalian cells. Certain vectors that can be used to express the antibody and the antibody fragment of the invention include plasmids that contain regulatory sequences (such as promoter and enhancer regions) that direct transcription of the gene. Other useful vectors for expressing the antibody and the antibody fragment contain polynucleotide sequences that enhance the rate of translation of these genes or improve the stability or nuclear export of mRNA transcripted from the genes. These sequence elements include, for example, 5′ and 3′ untranslated regions, internal ribosomal entry site (IRES), and polyadenylation signal site in order to direct the efficient transcription of the genes carried on the expression vector. The expression vector of the present invention may also contain a polynucleotide encoding a marker for selection of cells containing such a vector. Examples of suitable markers include genes encoding resistance to antibiotics such as ampicillin, chloramphenicol, kanamycin or nourseothyrcin.

The term “host cell” herein refers to a cell into which a foreign nucleic acid is introduced, including the progenys of such a cell. The host cell includes “transformant” and “transformed cell” which include the primary transformed cell and progeny derived therefrom, regardless of the number of passages. The progeny may not be identical to the parental cell in nucleic acid content, and may contain a mutation.

The mutant progeny with the same function or biological activity as those screened or selected in the initially transformed cells are included herein.

As used herein, the term “pharmaceutical composition” refers to a preparation that is present in a form which allows the active ingredients contained therein to be biologically effective and does not contain additional ingredients that would be unacceptably toxic to the subject to which the pharmaceutical composition is administered.

As used herein, the terms “subject”, “object” and “patient” refer to an organism receiving treatment for a particular disease or condition, such as a cancer or an infectious disease, as described herein. Examples of subjects and patients include mammals, such as humans, primates, pigs, goats, rabbits, hamsters, cats, dogs, guinea pigs, members of the bovid family (cattle, bison, buffalo, elk, yak, etc.), sheep, and horses receiving treatment for a disease or a condition (for example, a cell proliferative disorder, such as a cancer or an infectious disease).

As used herein, the term “treatment” refers to surgical or therapeutic treatment, the purpose of which is to prevent, slow down (reduce) an undesired physiological change or pathology in the subject being treated, such as the progress of a cell proliferative disorder (such as a cancer or an infectious disease). Beneficial or desired clinical results include, but are not limited to, alleviation of symptoms, diminishment of extent of disease, stabilized (i.e., not worsening) state of disease, delay or slowing of disease progression, amelioration or palliation of the disease state and remission (whether partial response or complete response), whether detectable or undetectable. Subjects in need of treatment include subjects who already have a condition or a disease, subjects who are prone to a condition or a disease or subjects who intend to prevent a condition or a disease. When referring to the terms slow down, alleviation, diminishment, palliation, remission, etc., the meaning of eliminate, disappear, not occur, etc. is also included.

The term “effective amount” herein refers to an amount of a therapeutic agent effective to prevent or relieve a disease or a condition or the progression of the disease when administered alone or in combination with another therapeutic agent to a cell, tissue or subject. “Effective amount” also refers to an amount of a compound sufficient to relieve symptoms, for example, treat, cure, prevent or relieve the associated medical conditions, or to increase the rate of treatment, cure, prevent or relieve such conditions. When the active ingredient is administered to an individual alone, a therapeutically effective dose refers to the ingredient alone. When a combination is used, a therapeutically effective dose refers to the combined amounts of the active ingredients that produce a therapeutic effect, whether administered in combination, sequentially or simultaneously.

The term “appropriate condition” herein refers to a condition suitable for culturing various host cells, including eukaryotic cells and prokaryotic cells.

The term “cancer” herein refers to or describes the physiological condition in mammals that is typically characterized by unregulated cell growth. Both benign and malignant cancers are included in this definition.

The term “tumor” herein refers to all neoplastic cell growth and proliferation, whether malignant or benign, and to all pre-cancerous and cancerous cells and tissues. The terms “cancer” and “tumor” are not mutually exclusive when referred to herein.

The term “anti-tumor agent” herein refers to an anti-tumor drug, which is a class of drugs for the treatment of tumor diseases, for example, a chemotherapy drug, a biological agent and the like.

The term “EC50” herein refers to the half-maximal effective concentration, which includes the concentration of an antibody that induces a response halfway between baseline and maximum after a specified exposure time. EC50 essentially represents the concentration of an antibody at which 50% of its maximal effect is observed and which can be measured by methods known in the art.

### BRIEF DESCRIPTION OF THE DRAWINGS

Unless otherwise defined herein, scientific and technical terms related to the present invention shall have the meanings understood by those of ordinary skill in the art.

FIG. 1 shows the detection results of SDS-PAGE reducing and non-reducing gels of CD22-ECD-His, CD22 domain1-4-His and CD22 domain5-7-His protein samples. Lane 1 is the protein band of hCD22-ECD-His under non-reducing conditions, Lane 2 is the protein band of hCD22 domain5-7-His under non-reducing conditions, Lane 3 is the protein band of hCD22 domain5-7-His under reducing conditions, Lane 4 is the protein band of hCD22-ECD-His under reducing conditions, Lane 5 is the protein band of hCD22 domain1-4-His under non-reducing conditions, Lane 6 is the protein band of hCD22 domain1-4-His under reducing conditions, and Lane M is the protein marker band.

FIG. 2A is the FACS result of detecting CD22 expression quantity in Raji cells by HA22 antibody; FIG. 2B shows the FACS result of detecting CD22 expression quantity in Raji cells by m971 antibody.

FIG. 3A shows the FACS screen and detection results of CHO-K1-human CD22 2C4 cells transfected with human CD22 protein; FIG. 3B shows the FACS screen and detection results of CHO-K1-human CD22 1G5 cells transfected with human CD22 protein; FIG. 3C shows the FACS screen and detection results of CHO-K1-human CD22 1D9 cells transfected with human CD22 protein.

FIG. 4 shows the FACS result of detecting HEK293T cells transfected with monkey CD22 protein by hL22 antibody.

FIG. 5 shows the binding reaction of chimeric antibody with human CD22-ECD-His protein detected by ELISA. The anti-CD22 positive control antibody is: HA22 and m971, the negative control is hIgG1.

FIG. 6A shows the binding reaction of the chimeric antibody of the present invention with Raji detected by FACS; FIG. 6B shows the binding reaction of the chimeric antibody of the present invention with CHO-K1-human CD22 detected by FACS; The anti-CD22 positive control antibody is: HA22 and m971, the negative control is hIgG1; FIG. 6C shows the binding reaction of the chimeric antibody of the present invention at 1 nM and 10 nM with Raji cells and MOLT4 cells detected by FACS; FIG. 6D shows the binding reaction of the chimeric antibody of the present invention at 1 nM and 10 nM with CHO-K1 cells and CHO-K1-human CD22 cells detected by FACS.

FIG. 7 shows the binding reaction of the chimeric antibody of the present invention with murine CD22-ECD-HiS protein detected by ELISA; The positive control is 983; the negative control is hIgG1.

FIG. 8 shows the binding reaction of the chimeric antibody of the present invention with monkey CD22-ECD-His protein detected by ELISA; The positive control is HA22; the negative control is hIgG1.

FIG. 9A shows the binding reaction of the chimeric antibody of the present invention with HEK293T-monkey CD22 detected by FACS; The anti-CD22 positive control antibody is: HA22, the negative control is hIgG1; FIG. 9B shows the binding reaction of the chimeric antibody of the present invention at 1 nM and 10 nM with HEK293T cells and HEK293T-monkey-CD22 detected by FACS.

FIG. 10A-10B show a scatter plot of peripheral blood mononuclear cells of cynomolgus monkeys stained with both CD20 antibody and 1 nM of the chimeric antibody of the present invention detected by FACS, CD20 is a B cell marker, and the ratio shown in the figure is the ratio of chimeric antibody positive cells to CD20 positive cells, and the anti-CD22 positive control antibody is: HA22 and hL22, the negative control is hIgG1.

FIG. 11A shows the binding reaction of the chimeric antibody of the present invention with human CD22 domain1-4-His protein detected by ELISA; FIG. 11B shows the binding reaction of the chimeric antibody of the present invention with human CD22 domain5-7-His protein detected by ELISA. The anti-CD22 domain1-4 positive control antibody is HA22, the anti-CD22 domain5-7 positive control antibody is m971, and the negative control is hIgG1.

### DETAILED DESCRIPTION OF EMBODIMENTS

The present invention will be further described below in conjunction with specific examples, and the advantages and characteristics of the present invention will become clearer along with the description. If specific conditions are not specified in the examples, conventional conditions or conditions recommended by a manufacturer are followed. The reagents or instruments used therein for which manufacturers are not specified are all conventional products that are commercially available.

The examples of the present invention are merely exemplary, and do not limit the scope of the present invention in any way. Those skilled in the art should understand that the details and forms of the technical solutions of the present invention can be modified or replaced without departing from the spirit and scope of the present invention, but these modifications and replacements all fall within the protection scope of the present invention.

### EXAMPLE 1

### 1.1 Preparation of Human CD22-His Tag Protein

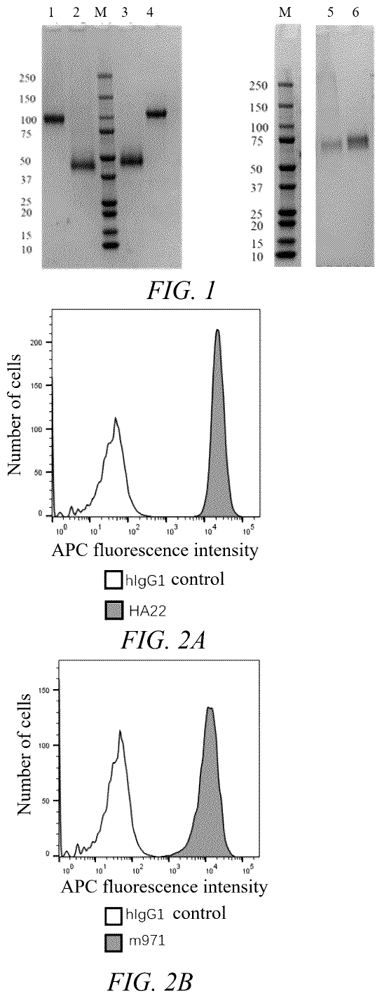

The CD22 protein has 7 IgG-like domains outside the cell, in which domain 1 is located at the farthest end from the membrane and domain 7 is located at the nearest end from the membrane. The nucleotide sequences encoding the amino acid sequence of human CD22 protein (NCBI: NP_001762.2, SEQ ID NO: 1), the extracellular region (ECD, extra-cellular domain) amino acid sequence Asp 20-Arg 687 (SEQ ID NO: 2), the domain 1-4 Asp 20-Val 425 amino acid sequence (SEQ ID NO: 3) and the domain 5-7 Asp 414-Arg 687 amino acid sequence (SEQ ID NO: 4) were cloned into the pTT5 vector by GENERAL Biosystems (Anhui) Corporation Limited, respectively, and plasmids were prepared according to the established standard molecular biology methods. The corresponding amino acid sequence information was shown in Table 1 below. For the specific method, see Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotechnology Co., Ltd.) were transiently transfected (PEI, Polysciences, catalog number: 24765-1) and FreeStyle™ 293 (Thermofisher scientific, catalog number: 12338018) was used for scale-up culture at 37° C. After 6 days, the cell culture fluid was collected, centrifuged to remove cell components, and the culture supernatant containing the extracellular region of human CD22 protein was obtained. The culture supernatant was loaded onto a nickel ion affinity chromatography column HisTrap™Excel (GE Healthcare, catalog number: GE17-3712-06), and the change in the ultraviolet absorbance (A280 nm) was monitored with an ultraviolet (UV) detector. After sample loading, the nickel ion affinity chromatography column was washed with 20 mM PB, 0.5M NaCl (pH 7.4) until the ultraviolet absorbance returned to the baseline, and then gradient elutions (2%, 4%, 8%, 16%, 50%, 100%) were performed with Buffer A: 20 mM PB, 0.5M NaCl (pH 7.4) and Buffer B: 20 mM PB, 0.5M NaCl, and 500 mM imidazole. His-tagged human CD22 protein eluted from the nickel ion affinity chromatography column was collected and dialyzed against PBS phosphate buffer (PH 7.4) overnight in a refrigerator at 4° C. The dialyzed protein was aseptically filtered by 0.22 micron filter membrane and then subpackaged for storage at −80° C. to obtain purified human CD22 protein. The bands of interest of samples detected by SDS-PAGE reducing gel and non-reducing gel were shown in FIG. 1.

TABLE 1
Amino acid sequences of human CD22 protein and extracellular region
of sequence

| Designation | | |
|---|---|---|
| of sequence | Sequence | Amino acid sequence |
| Human CD22 | SEQ ID NO: 1 | [symbol] |
| protein | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| Haman CD22 | SEQ ID NO: 2 | [symbol] |
| extracellular | | [symbol] |
| region | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| Human CD22 | SEQ ID NO: 3 | [symbol] |
| protein | | [symbol] |
| domain1-4 | | [symbol] |
| | | [symbol] |
| | | [symbol] |
| | | [symbol] |
| | | [symbol] |
| | | [symbol] |
| | | [symbol] |
| | | [symbol] |
| Human CD22 | SEQ ID NO: 4 | [symbol] |
| protein | | [symbol] |
| | | [symbol] |
| | | [symbol] |
| | | [symbol] |
| | | [symbol] |
| | | [symbol] |

[symbol]

indicates data missing or illegible when filed

### 1.2 Preparation of Human CD22 Control Antibody

HA22 and m971 clones were antibodies that recognize human CD22, wherein the antigen-binding epitope of the HA22 clone was located in domain 2-3, and the antigen-binding epitope of the m971 clone was located in domain5-7. The heavy chain variable region sequence and the light chain variable region sequence of the HA22 clone were obtained according to patent U.S. Pat. No. 9,580,461 B (which was incorporated herein by reference), and the heavy chain variable region sequence and the light chain variable region sequence of the m971 clone were obtained according to patent U.S. Pat. No. 8,591,889 B (which was incorporated herein by reference). Taizhou Biointron Biotechnology Co., Ltd. cloned the light chain variable region sequences of the HA22 and m971 into the expression vector pcDNA3.4-B1HH1 containing a signal peptide and the light chain constant region of human antibody IgG1 (private vector of Taizhou Biointron Biotechnology Co., Ltd.), respectively, and cloned the heavy chain variable region sequences into the expression vector pcDNA3.4-B1HLK containing a signal peptide and the heavy chain constant region of human antibody IgG1 (private vector of Taizhou Biointron Biotechnology Co., Ltd.), respectively, and thus sequences of m971-hIgG1 and HA22-hIgG1 were obtained. Unless otherwise specified, HA22 and m971 referred to m971-hIgG1 and HA22-hIgG1 respectively hereinafter. Plasmids were prepared according to the established standard molecular biology methods. For the specific method, see Sambrook, J., Fritsch, E. F., and Maniatis, T. (1989). Molecular Cloning: A Laboratory Manual, Second Edition (Plainview, New York: Cold Spring Harbor Laboratory Press). The expression vector was transiently transfected into HEK293E cells (purchased from Suzhou Yiyan Biotechnology Co., Ltd.) according to the instructions of PEI (purchased from Polysciences, catalog number: 24765-1), and the transfected cells were continuously cultured at 37° C. for 5 days using FreeStyle TM 293 (Thermo Fisher Scientific, catalog number: 12338018), and the cell components were removed by centrifugation to obtain the culture supernatant containing the antibody. The culture supernatant was loaded onto the protein A chromatography column (the protein A filler AT Protein A Diamond and the chromatography column BXK 16/26 were both purchased from Bestchrom (Shanghai) Biosciences Ltd., and the catalog numbers were: AA0273 and B-1620, respectively), the column was washed with PBS phosphate buffer (pH 7.4), then washed with 20 mM PB, 1M NaCl (pH 7.2), and finally eluted with citric acid buffer (pH 3.4). The antibody with Fc label eluted from the protein A chromatography column was collected, neutralized with 1/10 volume of 1M Tris (PH 8.0), and dialyzed with PBS at 4° C. overnight, and the dialyzed protein was aseptically filtered by 0.22 micron filter membrane and then subpackaged for storage at −80° C.

TABLE 2
Sequence information of heavy and light chains of anti-human CD22
antibodies HA22-hIgG1 and m971-hIgG1

| Designation | Sequence | |
|---|---|---|
| of sequence | number | Amino acid sequence |
| HA22-hIgH1 | SEQ ID | [symbol] |
| heavy chain | NO: 5 | [symbol] |
| | | [symbol] |
| | | [symbol] |
| | | [symbol] |
| | | [symbol] |
| | | [symbol] |
| | | [symbol] |
| | | [symbol] |
| | | [symbol] |
| HA22-hIgG1 | SEQ ID | [symbol] |
| light chain | NO: 6 | [symbol] |
| | | [symbol] |
| | | [symbol] |
| | | [symbol] |
| m971-hIgG1 | SEQ ID | [symbol] |
| heavy chain | NO: 7 | [symbol] |
| | | [symbol] |
| | | [symbol] |
| | | [symbol] |
| | | [symbol] |
| | | [symbol] |
| | | [symbol] |
| | | [symbol] |
| | | [symbol] |
| m971-hIgG1 | SEQ ID | [symbol] |
| light chain | NO: 8 | [symbol] |
| | | [symbol] |
| | | [symbol] |
| | | [symbol] |

[symbol]

indicates data missing or illegible when filed

### EXAMPLE 2

### Construction and Identification of Cell Lines Overexpressing Human CD22 and Monkey CD22

### 2.1 Identification of Cell Line Expressing CD22 Endogenously

Raji cells (purchased from China Center for Type Culture Collection, Wuhan University, catalog number: TCHu 44) were scale-up cultured in T-25 cell culture flasks to the logarithmic growth phase, the supernatant of the medium was discarded by centrifugation, and the cell pellet was washed 2 times with PBS. The HA22 and m971 antibody were used as primary antibodies, APC-labeled secondary antibody (purchased from Biolegend, catalog number: 409306) was used and FACS (FACS Canto™, purchased from BD company) was used for detection and result analysis. The analysis results were shown in Table 3 and FIGS. 2A-2B, the Raji cells can bind to HA22 and m971.

TABLE 3
FACS detection results of the endogenous cell line Raji cells

Cell mean fluorescence
density

| | Designation of | IgG subtype | CD22 |
|---|---|---|---|
| No. | antibody | control | antibody |
| 1 | HA22 | 44 | 22952 |
| 2 | m971 | 44 | 12686 |

### 2.2 Preparation of CHO-K1 Monoclonal Cell Line Stably Transfected With Human CD22

The nucleotide sequence encoding the full-length amino acid sequence of human CD22 (NCBI: NP_001762.2, SEQ ID NO: 1) was cloned into the pcDNA3.1 vector and a plasmid was prepared by GENERAL Biosystems (Anhui) Corporation Limited. Plasmid transfection (Lipofectamine® 3000 Transfection Kit, purchased from Invitrogen, catalog number: L3000-015) was performed on CHO-K1 cell line (purchased from Shanghai Institutes for Biological Sciences, Chinese Academy of Sciences, catalog number: SCSP-507), and then the transfected cells were selectively incubated for 2 weeks in DMEM/F12 medium containing 10 μg/ml of puromycin and 10%(w/w) of fetal bovine serum. The FITC-labeled anti-CD22 antibody (Thermofisher scientific, catalog number: 11-0229-42) was used to sort the positive monoclonal cells into a 96-well plate on flow cytometer FACS AriaII (BD Biosciences) and the plate was placed in a cell incubator at 37° C. and 5% (v/v) CO₂ for cell culture. Some wells containing monoclonal cells were selected for amplification after approximately 2 weeks. The amplified clones were screened by flow cytometry. The monoclonal cell line with better growth and higher fluorescence intensity was selected for further scale-up culture and then freezed in liquid nitrogen.

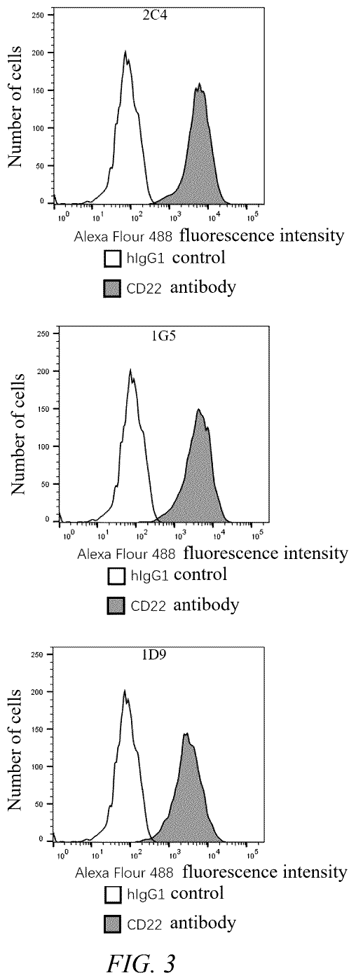

The specific selection results were shown in Table 4 and FIG. 3A-FIG. 3C, and the IgG subtype control was human IgG1 control. Table 4 showed that a series of CHO-K1 monoclonal cell lines positive for CD22 expression was prepared. In FIG. 3A-FIG. 3C, the abscissa was the fluorescence intensity of cells, and the ordinate was the number of cells. The results in FIG. 3A-FIG. 3C indicated that CHO-K1-human CD22 2C4, CHO-K1-human CD22 1G5 and CHO-K1-human CD22 1D9 were cell lines with high expression of CD22. CHO-K1-human 2C4 cell line was finally selected for the following examples.

TABLE 4
FACS detection results of CHO-K1 cell line
stably transfected with human CD22 protein

Cell mean fluorescence
intensity

| | Clone number of stably | IgG subtype | CD22 |
|---|---|---|---|
| No. | transfected cell line | control | antibody |
| 1 | CHO-K1-A CD22 2C4 | 66 | 4621 |
| 2 | CHO-K1-A CD22 1G5 | 66 | 3154 |
| 3 | CHO-K1-A CD22 1D9 | 66 | 2488 |

### 2.3 Preparation of HEK293T Cell Line Stably Transfected With Monkey CD22

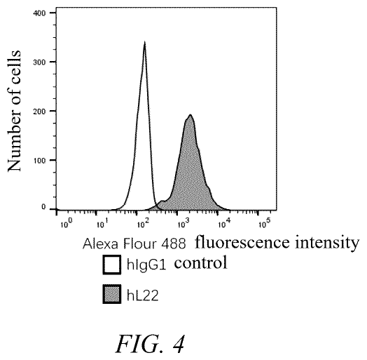

The nucleotide sequence encoding the full-length amino acid sequence of the monkey CD22 (NCBI: XP_014979161.2, SEQ ID NO: 9) was cloned into the pcDNA3.1 vector (purchased from Thermofisher scientific, catalog number: V79020) and a plasmid was prepared. After plasmid transfection of the HEK293T cell line with FuGENE® HD (Promega, catalog number: #E2311), the transfected cells were selectively cultured in DMEM medium containing 10 μg/ml puromycin and 10%(w/w) fetal bovine serum for 2 weeks, subcloned in 96-well culture plates by limited dilution method, and cultured in a 37° C., 5% (v/v) CO₂ incubator. After about 2 weeks, some wells contained polyclones were selected and amplification was performed into 6-well plates. The amplified clones were screened by CD22 antibody hL22 with monkey cross activity (purchased from Enzo Life Sciences; catalog number: ENZ-ABS619-0200) by flow cytometry analysis, the cell line with better growth and higher fluorescence intensity was selected for further scale-up culture and then freezed and stored in liquid nitrogen. FIG. 4 is the result of flow cytometry analysis of the HEK293T cell line detected by hL22 antibody. The result shows a single positive cell peak overexpressing monkey CD22 after puromycin screening, which could be used to detect the cross-activity of antibodies.

Monkey CD22 full-length amino acid sequence
(SEQ ID NO: 9)
MHLLGPWLLLLVLEYLAFSDSSKWNIEHPGTIYAWEGACVWVPCTYRVLDGALETFILFHNPEYNQNMSKFEGTRL
YESTKDGKLPSGQKRVQRLGNKINNNCTLSHIPVHVNDSGQLGLRMVSKTEKWMERHILNVSERPFPPRIQLPPKLQESQ
EVTLTCLLNFSCYGYQIQLQWLLEGVPMRQAAVTLTSLSTKSVFTRSELKFSPQWSHHGKIVTCELHDVDGKVLSEDMVQ
LNVKHTPKLTIEVTPNETTVRKGDSVTMTCKVNSSNPEYTTVSWLKDGIPLKEQNTLMLTLHKVTKSQSGRYCCRVSNDV
GPATSEKVFLQVQYAPESSRVQISQSPAVEGSEVNFLCISPANPLPTNYTWYHNGKEVQGRTEKQFQIQKILPWHAGTYS
CEAENILGIGERGPGTELDVQYPPKKVTMVIENPTPIREGDTVTLSCNYSSSNPIVNHYEWRPRGAWEEPSLGVLKIQNI
GWNNTAVACAACNNWCSWASPVTLNVLYAPRGVRVRKTKPLSETHSGNSVSLQCDESSSHPKEVQFFWEKNGSLLGKESQ
LNFDSISPEDAGSYSCWVNNSIGQTASKAWTLEVLYAPRRLRVSMSQGNQVMEGKTATLICESDANPPVYSYAWFDWNVQ
SLPYSGRMLRLEPVKVQHSGAYWCQGTNRVGKGHSPLITLTVYYSPQTIGRRVAVGLGSCLAILILAMCGFKVQRRWKRT
QSQQGLQENSSGQSFFVRNKKVRRTPLSEGPHSLGCYNPMMEDGISYATLRFPETNTPRTGDAETSELQRPPPDCDDTVT
YSVLQKRQVGDYENVTPDFPEDEGTHYSELTQFGFGERPQAQENVDYVTVKH

### EXAMPLE 3

### Preparation of Anti-Human CD22 Hybridoma Monoclonal Antibody

### 3.1 Animal Immunity

An anti-human CD22 monoclonal antibody was produced by immunizing mice. BALB/c AnNCrl mice and SJL/JorllcoCrl mice (purchased from Shanghai SLAC Co., Ltd.), female, aged 6-8 weeks, were used in the experiment. Breeding environment: SPF rating. After the mice were purchased, they were raised in a laboratory environment for 1 week, with a 12/12-hour light/dark cycle adjustment, and a temperature of 20-25° C.; humidity 40%-60%. The acclimatized mice were immunized according to the following scheme. The immune antigen was human CD22 (Asp20-Arg687)-His protein (purchased from ACRO Biosystems, catalog number: CD2-H52H8). For the first immunization, the immunogen was emulsified with TiterMax (purchased from Sigma, catalog number: T2684) and injected subcutaneously and intraperitoneally at 0.1 ml respectively, that is, each mouse was injected with 50 micrograms of immunogen protein A. For booster immunization, the immunogen was injected subcutaneously and intraperitoneally at 0.1 ml with Imject Alum Adjuvant (purchased from Thermofisher scientific, catalog number: 77161), that is, each mouse was injected with 25 micrograms of the immunogen. The frequency of immunization was once a week, blood was collected on the 4th, 18th, 46th, and 70th day, and the antibody titer in the mouse serum was detected by ELISA and FACS methods. The results are shown in Table 5-8. After immunization, the sera of mice immunized with human CD22-his protein binded to the immunogen to varying degrees, showing antigen-antibody reactions, and the highest dilution was about 6 million. The blank control is 1% (w/w) BSA, the batch refers to the mouse serum on the seventh day after the seventh booster immunization, and the data in the table are OD450 nm and MFI values.

TABLE 5
Serum antibody titer of Balb/c mice after immunization detected by ELISA

| | OD450 nm | | | | | | | | | | | |
|---|---|---|---|---|---|---|---|---|---|---|---|---|
| | Serum dilution | | | | | | | | | | | |
| | | | | | | | | | | | | Blank |
| Batch | 1:100 | 1:300 | 1:900 | 1:2700 | 1:8100 | 1:24300 | 1:72900 | 1:218700 | 1:656100 | 1:1968300 | 1:5904900 | control |
| 261 (TB3) | 2.55 | 2.60 | 2.57 | 2.54 | 2.25 | 2.10 | 1.69 | 1.23 | 0.65 | 0.33 | 0.20 | 0.09 |
| 262 (TB3) | 2.33 | 2.26 | 2.22 | 2.27 | 2.07 | 1.94 | 1.51 | 0.95 | 0.45 | 0.23 | 0.13 | 0.08 |
| 263 (TB3) | 2.40 | 2.24 | 2.15 | 2.13 | 2.01 | 1.82 | 1.41 | 0.85 | 0.41 | 0.20 | 0.12 | 0.08 |
| 264 (TB3) | 2.40 | 2.31 | 2.30 | 2.27 | 2.07 | 1.82 | 1.30 | 0.71 | 0.31 | 0.16 | 0.10 | 0.07 |
| 265 (TB3) | 2.35 | 2.31 | 2.32 | 2.16 | 2.08 | 1.84 | 1.41 | 0.85 | 0.41 | 0.19 | 0.11 | 0.08 |

TABLE 6
Serum antibody titer of SJL mice after immunization detected by ELISA

| | OD450 nm | | | | | | | | | | | |
|---|---|---|---|---|---|---|---|---|---|---|---|---|
| | Serum dilution | | | | | | | | | | | |
| | | | | | | | | | | | | Blank |
| Batch | 1:100 | 1:300 | 1:900 | 1:2700 | 1:8100 | 1:24300 | 1:72900 | 1:218700 | 1:656100 | 1:1968300 | 1:5904900 | control |
| 266 (TE3) | 2.40 | 2.36 | 2.35 | 2.24 | 2.05 | 1.79 | 1.34 | 0.75 | 0.37 | 0.19 | 0.12 | 0.08 |
| 267 (TB3) | 2.31 | 2.16 | 2.02 | 1.82 | 1.51 | 1.06 | 0.53 | 0.26 | 0.13 | 0.09 | 0.07 | 0.09 |
| 268 (TB3) | 2.29 | 2.10 | 1.96 | 1.64 | 1.29 | 0.78 | 0.35 | 0.19 | 0.12 | 0.10 | 0.10 | 0.11 |
| 269 (TB3) | 2.61 | 2.49 | 2.36 | 2.29 | 2.07 | 1.88 | 1.45 | 0.88 | 0.43 | 0.21 | 0.13 | 0.09 |
| 270 (TB3) | 1.83 | 1.23 | 0.63 | 0.28 | 0.14 | 0.08 | 0.08 | 0.06 | 0.09 | 0.09 | 0.07 | 0.08 |

TABLE 7
Serum antibody titer of Balb/c mice after immunization detected by FACS

| | MFI | | | | | | | | | | | |
|---|---|---|---|---|---|---|---|---|---|---|---|---|
| | Serum dilution | | | | | | | | | | | |
| | | | | | | | | | | | | Blank |
| Batch | 1:100 | 1:300 | 1:900 | 1:2700 | 1:8100 | 1:24300 | 1:72900 | 1:218700 | 1:656100 | 1:1968300 | 1:5904900 | control |
| 261 (TB3) | 64458 | 52926 | 45069 | 39113 | 32293 | 20440 | 9577 | 4202 | 1860 | 821 | 459 | 131 |
| 262 (TB3) | 63113 | 54421 | 48096 | 41627 | 30655 | 17728 | 7976 | 3348 | 1476 | 679 | 399 | 112 |
| 263 (TB3) | 68470 | 54517 | 46623 | 39177 | 29120 | 16910 | 7792 | 3301 | 1374 | 641 | 340 | 89 |
| 264 (TB3) | 62190 | 48925 | 40670 | 32314 | 19921 | 9761 | 3979 | 1703 | 698 | 347 | 292 | 150 |
| 265 (TB3) | 63768 | 52435 | 45919 | 37727 | 26351 | 14061 | 6023 | 2590 | 1122 | 504 | 284 | 110 |

TABLE 8
Serum antibody titer of SJL mice after immunization detected by FACS

| | MFI | | | | | | | | | | | |
|---|---|---|---|---|---|---|---|---|---|---|---|---|
| | Serum dilution | | | | | | | | | | | |
| | | | | | | | | | | | | Blank |
| Batch | 1:100 | 1:300 | 1:900 | 1:2700 | 1:8100 | 1:24300 | 1:72900 | 1:218700 | 1:656100 | 1:1968300 | 1:5904900 | control |
| 266 (TB3) | 67436 | 53379 | 45330 | 37604 | 26313 | 13406 | 5887 | 2427 | 1095 | 514 | 310 | 195 |
| 267 (TB3) | 37379 | 27314 | 14870 | 6493 | 2661 | 1102 | 496 | 245 | 157 | 209 | 98 | 116 |
| 268 (TB3) | 34130 | 26195 | 14039 | 5895 | 2365 | 971 | 449 | 270 | 140 | 136 | 134 | 156 |
| 269 (TB3) | 9657 | 4490 | 1894 | 797 | 398 | 258 | 127 | 133 | 160 | 97 | 165 | 139 |
| 270 (TB3) | 12544 | 5962 | 2715 | 1054 | 494 | 261 | 182 | 102 | 129 | 113 | 142 | 117 |

After the 7th to 8th immunization, the mice with high antibody titer in serum which tended to plateau were selected for splenocyte fusion. Immunization was boosted 3 days before splenocyte fusion, an antigen solution prepared with normal saline was injected subcutaneously and intraperitoneally (IP) at 50 μg/mouse.

### 3.2 Splenocyte Fusion and Hybridoma Screening

ACK Lysing Buffer (purchased from Gibco, catalog number: A1049201) was added to lyse the red blood cells doped in the splenocytes to obtain a splenocyte suspension. The cells were washed with DMEM (purchased from Gibco, catalog number: 11995081) basal medium 3 times by centrifugation at 1000 rpm, and then mixed with mouse myeloma cells SP2/0 (purchased from ATCC, catalog number: CRL-1581) at a ratio of 2:1 in terms of living cells. BTX ECM2001+ high-efficiency electrofusion method (see METHODS IN ENZYMOLOGY, VOL. 220) was used for cell fusion. The fused cells were diluted into DMEM medium containing 20% fetal bovine serum (ExCell Bio, catalog number: FSD500), 1× HAT (purchased from Sigma, catalog number: H0262) (the percentages were mass percentages), then added into a 96-well cell culture plate at 2×10⁴/200 microliters per well, and put in 5% CO₂, 37° C. incubator (the percentage is a volume percentage). After 14 days, ELISA was used to screen the supernatant in the cell fusion plate, ELISA positive clones were amplified into a 24-well plate, and DMEM (purchased from Gibco, catalog number: 11995081) containing 10% (w/w) HT (purchased from Sigma, catalog number: H0137) and fetal bovine serum was used for scale-up culture at 37° C., 5% (v/v) CO₂. After culturing for 3 days, the culture medium of the scale-up culture in the 24-well plate was taken for centrifugation, and the supernatant was collected. the antibody subtype was analyzed for the supernatant, and ELISA and FACS were used to determine the binding activity to human CD22 protein and human CD22 positive cells (for the detection methods of binding activity, please refer to Example 5.1 and Example 5.2 respectively).

According to the screening results of the 24-well plate, the positive hybridoma cells in the ELISA and FACS experiments were selected as eligible positive clones, and were subcloned in DMEM medium containing 10% (w/w) FBS (purchased from Gibco, catalog number: 11995081) in a 96-well plate by limited dilution method, and cultured at 37° C. and 5% (v/v) CO₂. 10 days after subcloning, ELISA and FACS were used for preliminary screening, and a single positive clone was selected and amplified into a 24-well plate for further culture. According to the detection results of the samples in the 24-well plate, the optimal clone was selected and placed in DMEM medium containing 10% (w/w) FBS (purchased from Gibco, catalog number: 11995081) at 37° C. and 5% (v/v) CO₂ condition for scale-up culture. The obtained cells were frozen in liquid nitrogen to obtain the hybridoma cells of the present invention.

### EXAMPLE 4

### Determination of Amino Acid Sequence of Variable Regions of Light and Heavy Chains of Positive Hybridoma Clones

The hybridoma cells in the logarithmic growth phase were collected, and the cells were fully lysed with Trizol (Invitrogen, catalog number: 15596-018) and stored at −80° C. for testing. Suzhou GENEWIZ Biotechnology Co., Ltd. was entrusted to complete the determination of the amino acid sequences of the light and heavy chain variable regions of hybridoma positive clones. The sequencing results were analyzed by MOE software, and the phylogenetic tree was constructed according to the amino acid sequence of the protein encoded by the variable region. After eliminating the sequences that were close to each other on the phylogenetic tree according to the sequence similarity, 35 clones were obtained. Among them, there are 23 (F1.236.15, F1.214.8, F1.273.12, F1.231.15, F1.11.7, F1.77.9, F1.105.11, F1.267.9, F1.7.6, F1.224.1, F1.250.16, F1.120.15, F1.216.2, F1.280.1, F1.200.11, F1.192.1, F1.245.2, F1.60.9, F1.172.13, F1.17.1, F1.161.7, F1.257.3 and F1.62.10) for F1 series, and 12 (F2.70.2, F2.104.10, F2.180.16, F2.121.9, F2.173.9, F2.343.16, F2.205.9, F2.99.1, F2.127.11, F2.55.1, F2.42.9 and F2.151.13) for F2 series.

Taizhou Biointron Biotechnology Co., Ltd. cloned the heavy chain variable region sequences of the 35 clones into the expression vector pcDNA3.4-B1HH1 containing a signal peptide and human antibody IgG1 heavy chain constant region (SEQ ID NO: 10) (private vector of Taizhou Biointron Biotechnology Co., Ltd.), cloned the light chain variable region sequences of the clones of the F1 series into the expression vector pcDNA3.4-B1HLK containing a signal peptide and human antibody IgG1 Kappa light chain constant region (SEQ ID NO: 11) (private vector of Taizhou Biointron Biotechnology Co., Ltd.), cloned the light chain variable region sequences of the clones of the F2 series into the expression vector pcDNA3.4-BIHLS containing a signal peptide and human antibody IgG1 Lambda light chain constant region (SEQ ID NO: 12) (private vector of Taizhou Biointron Biotechnology Co., Ltd.), and the expression vector of human-murine chimeric antibody was obtained and the antibody was prepared according to the method of Example 1.2. The CDRs of the antibody sequences were analyzed by KABAT, Chothia or IMGT software respectively, and the corresponding sequence information is shown in the following Tables 9-10. Among them, Table 9 shows the antibody sequences represented by amino acids of the heavy and light chain variable regions of the 35 chimeric antibody molecules, and Table 10 shows the analysis results of IMGT, Kabat and Chothia of the CDRs of the 35 chimeric antibody molecules.

TABLE 9
Specific amino acid sequence information of the heavy chain variable
region and light chain variable region of the anti-CD22 antibody

| Antibody | Sequence | |
|---|---|---|
| number | number | Heavy chain variable region sequence (VH) |
| [symbol] | SEQ ID NO. 13 | [symbol] |
| | | [symbol] |
| | | [symbol] |
| [symbol] | SEQ ID NO. 15 | [symbol] |
| | | [symbol] |
| | | [symbol] |
| [symbol] | SEQ ID NO. 17 | [symbol] |
| | | [symbol] |
| | | [symbol] |
| [symbol] | SEQ ID NO. 19 | [symbol] |
| | | [symbol] |
| | | [symbol] |
| [symbol] | SEQ ID NO. 21 | [symbol] |
| | | [symbol] |
| | | [symbol] |
| [symbol] | SEQ ID NO. 23 | [symbol] |
| | | [symbol] |
| | | [symbol] |
| [symbol] | SEQ ID NO. 25 | [symbol] |
| | | [symbol] |
| | | [symbol] |
| [symbol] | SEQ ID NO. 27 | [symbol] |
| | | [symbol] |
| | | [symbol] |
| [symbol] | SEQ ID NO. 29 | [symbol] |
| | | [symbol] |
| | | [symbol] |
| [symbol] | SEQ ID NO. 31 | [symbol] |
| | | [symbol] |
| | | [symbol] |
| [symbol] | SEQ ID NO. 33 | [symbol] |
| | | [symbol] |
| | | [symbol] |
| [symbol] | SEQ ID NO. 35 | [symbol] |
| | | [symbol] |
| | | [symbol] |
| [symbol] | SEQ ID NO. 37 | [symbol] |
| | | [symbol] |
| | | YCVRGDYYNSRYWYFDVWGTGTTVTVSS |
| F1.120.15 | SEQ ID NO. 39 | QVQLQQSGPELVKPGASVKISCKASGNAFSNSWMNWVKQRPGKGLE |
| | | WIGRVYSEDGDTQYNGKFRDKATLTADRSTSTAYMQLSSLTSEDSAV |
| | | YFCARWLIYYGTYGAMDYWGQGTSVTVSS |
| F1.62.10 | SEQ ID NO. 41 | QVQLQQSGPELVKPGASVKISCKASGYEFSSIWMNWVKQRPGEGLE |
| | | WIGRIFPGDGEINYNEKFKGKATLTADRSSTTAYMQLSSLTSEDSAVY |
| | | FCAGWRIYYGTYGAMDYWGQGTSVTVSS |
| F1.216.2 | SEQ ID NO. 43 | QVQLQQSGPEVVRPGASVKISCKASGYAFSSSWMNWMKQRPGKALE |
| | | WIGRIYPGDGDTNYSGEFKVKATLTADKSSSTAYMQLSSLTSEDSAV |
| | | YFCAREGSYYSNPWYFDYWGQGTTLTVSS |
| F1.280.1 | SEQ ID NO. 45 | QIQLVQSGPELKKPGETVKISCKASGYTFTTYGMSWVKQTPGKGLTW |
| | | MGWINTDSGVPTYADDFKRRFAFSLEASATTAYLQINNLKNEDTATY |
| | | FCARGGPDYWGQGTTLTVSS |
| F1.200.11 | SEQ ID NO. 47 | QNQLVQSGPELKKPGETVKISCKASGYTFTTAGMQWVQKMPGKGFK |
| | | WIGWINTLSGEPKYAEDFKGRFAFSLETSATTAYLQISNLKNEDTATY |
| | | FCARAYYSNLYWYFDVWGTGTTVTVSS |
| F1.192.1 | SEQ ID NO. 49 | AVQLQQSGVELVRPGASVKLSCTGSDFNIKEDYIHWVKQRPEQGLEW |
| | | IGWIDPENGDTEYASKFQGKATITADTSSNTAYLQLSSLTSEDTAVYY |
| | | CATYGSLDYWGQGTTLTVSS |
| F1.245.2 | SEQ ID NO. 51 | EVQLQQSGAELVKPGASVKLSCTGSGFNIKDYYIHWVKQRTEQGLEW |
| | | MGRIDPEDGETEYAPKFQDEATITSDTSSNTAYLQLSSLTYEDTAVYY |
| | | CAREAAGYFYRGSSYGYFDVWGTGTTVTVSS |
| F1.60.9 | SEQ ID NO. 53 | QVQLKESGPGLVAPSQSLSITCTVSGLSLNNYGVSWVRQPPGKGLEW |
| | | LGVIWGDGSTNYHSALISRLSISKDNSKSQVFLKLNSLHTDDTATYYC |
| | | AINWGDYWGQGTTLIVSS |
| F1.172.13 | SEQ ID NO. 55 | QVTLKESGPGILQSSQTLSLTCSFSGFSLSTSGMGVSWIRQPSGKGLEW |
| | | LAHIYWDDDKRYNPSLKSRLTISKDTSRNQVFLKITSVDTADTATYYC |
| | | ARAPPPNWDEYYFDYWGQGTTLTVSS |
| F1.17.1 | SEQ ID NO. 57 | QVQLKESGPGLVAPSQSLSITCTVSGFSLSRYTVHWVRQPPGKGLEWL |
| | | GMIWGGGSTDYNSALKSRLSISKDNSKSQVFLKMNSLQTDDTAMYY |
| | | CARPHDFDAGGFAYWGQGTLVTVSA |
| F2.151.13 | SEQ ID NO. 59 | QVHLQQPGTELMKPGASVKLSCKATGYTFRDYWLEWVKQRPGHGL |
| | | EWIGEILPGSGNAYYNEKFKGKATFTADTSSNTAYMQLSSLTTEDSAI |
| | | YYCARVYSNWYFDAWGKGTTVTVSS |
| F2.70.2 | SEQ ID NO. 61 | EVQLVESGGGLVKPGGSLKLSCAASGFTFSDYGMNWFRQAPEKGLE |
| | | WVAYISRGSHTIYYADTVKGRFTISRDNAKNTLFLQMTSLRSNDTAM |
| | | YYCVRMAGYYAMDYWGQGTSVTVSS |
| F2.104.10 | SEQ ID NO. 63 | EVQLVESGGGFVKPGGSLKLSCAASGFTFSDYGMNWFRQAPEKGLE |
| | | WVAYISRGSHTIYYADTVKGRFTISRDNAENTLFLQMTSLRSNDTAM |
| | | YYCVRMAGYYAMDYWGQGTSLTVSS |
| F2.180.16 | SEQ ID NO. 65 | EVKLVESGGGLVLPGGSLKISCAASGFTFSDYYMYWIRQTPDKRLEW |
| | | VAYISNTGYSTFYPDSVKGRFTVSRDNAKNTLYLQMSRLQSEDTAMY |
| | | YCARHGILYAMDYWGQGTSVTVSS |
| F2.121.9 | SEQ ID NO. 67 | EVQLVESGGGLVKPGGSLKLSCAASGFTFSDYVMSWIRQTPEKRLEW |
| | | VATISDAGSYTFYPDNLKGRFTISRDNAKNNLYLQMNHLTSEDTAFY |
| | | YCAITYFGNYGGYWGQGTLVTVSV |
| F2.173.9 | SEQ ID NO. 69 | EVQLVESGGDLVKPGESLKLSCEASGFTFSNYAMSWVRQTPDRRLEW |
| | | VATISSIGSFTYYSDSVKGRFTISRDNVKSTLHLQMNNLKSGDTAIYFC |
| | | [symbol] |
| [symbol] | SEQ ID NO. 71 | [symbol] |
| | | [symbol] |
| | | [symbol] |
| [symbol] | SEQ ID NO. 73 | [symbol] |
| | | [symbol] |
| | | [symbol] |
| [symbol] | SEQ ID NO. 75 | [symbol] |
| | | [symbol] |
| | | [symbol] |
| [symbol] | SEQ ID NO. 77 | [symbol] |
| | | [symbol] |
| | | [symbol] |
| [symbol] | SEQ ID NO 79 | [symbol] |
| | | [symbol] |
| | | [symbol] |
| [symbol] | SEQ ID NO. 81 | [symbol] |
| | | [symbol] |
| | | [symbol] |
| Antibody | Sequence | |
| number | number | Light chain variable region sequence (VL) |
| [symbol] | SEQ ID NO. 14 | [symbol] |
| | | [symbol] |
| | | [symbol] |
| [symbol] | SEQ ID NO. 16 | [symbol] |
| | | [symbol] |
| | | [symbol] |
| [symbol] | SEQ ID NO. 18 | [symbol] |
| | | [symbol] |
| | | [symbol] |
| [symbol] | SEQ ID NO. 20 | [symbol] |
| | | [symbol] |
| | | [symbol] |
| [symbol] | SEQ ID NO. 22 | [symbol] |
| | | [symbol] |
| | | [symbol] |
| [symbol] | SEQ ID NO. 24 | [symbol] |
| | | [symbol] |
| | | [symbol] |
| [symbol] | SEQ ID NO. 26 | [symbol] |
| | | [symbol] |
| | | [symbol] |
| F1 257.3 | SEQ ID NO. 28 | [symbol] |
| | | [symbol] |
| | | [symbol] |
| F1.105.11 | SEQ ID NO. 30 | [symbol] |
| | | [symbol] |
| | | [symbol] |
| F1.267.9 | SEQ ID NO. 32 | DIVLTQSPASLAVSLGQRATISCRASESVDNYGFDFIHWYQQKPGQPP |
| | | KLLIYRASNLESGIPARFSGSGSRPDFTLTINPVETDDVATYYCQQSIKD |
| | | PWTFGGGTKLEIK |
| F1.7.6 | SEQ ID NO. 34 | DIVMSQSPSSLAVSVGEKVTMSCKSSQSLLYSNNQKNYLAWYQQKPG |
| | | QSPKKLIYWASTRESGVPDRLTGSGSGTDFTLTISSVTAEDLAVYYCQ |
| | | QFYSYPWTFGGGTKLEIK |
| F1.224.1 | SEQ ID NO. 36 | DIVLTQSPASLAVSLGQRATISCRASQSVSTPRYSFMHWYQQKPGQPP |
| | | KLLIKYASNLESGFPARFTGSGSGTDFTLNIHPVEEEDTAAYYCQQSW |
| | | AIPWTFGGGTKLEIK |
| F1.250.16 | SEQ ID NO. 38 | DIVMTQSQKFMSTTVGDRVSITCKAGQNVRTAVAWYQQKPGQSPKL |
| | | LIYSASTRYIGVPERFTGSGSGTDFTLTITNMQSEDLADYFCQQYSSFP |
| | | LTFGTGTKLELK |
| F1.120.15 | SEQ ID NO. 40 | DIQMTQTTSSLSASLGDRVTISCRASQDINNYLNWYHQKPDGTVKLLI |
| | | YFTSRPYSGVPSRFSGSGSGTDYSLTISNLEQEDIATYFCQQGNTLPWT |
| | | FGGGTKLEIK |
| F1.62.10 | SEQ ID NO. 42 | DIQMTQITSSLSASLGDRVTISCRASQDIGNYLNWYQQKPDGTVKLLI |
| | | YYTSRLHSGVPSRFSGSGSRTDYSLTISNLQQEDIATYFCQQGNTLPWT |
| | | FGGGTKLEIK |
| F1.216.2 | SEQ ID NO. 44 | DVLVTQTPLSLPVSLGDQASISCRSSQSIVQSNGDTYLEWYLQKPGQS |
| | | PKLLIYKVSNRFSGVPDRFSGSGSGTDFTLKISRVEAEDLGVYYCFQGS |
| | | HVPFTFGSGTKLEIK |
| F1.280.1 | SEQ ID NO. 46 | DILLTQSPATLSVTPGETVSLSCRASQSIYKNLHWYHQKSHRSPRLLIK |
| | | YASDSISGIPSRFTGSGSGTNFTLSINSVKPEDEGIYYCLQGYIVPLTFG |
| | | AGTKLELK |
| F1.200.11 | SEQ ID NO. 48 | DIVMTQSQKFMSTTVGDRVTITCKASQNVVTAVAWYQQKPGQSPKV |
| | | LIYSASNRYSGVPDRFTGSGSGTDFTLTISNMHSEDLANYFCHQYSSYP |
| | | FTFGSGTKLEIK |
| F1.192.1 | SEQ ID NO. 50 | DIKMTQSPSSMYAFLGERVTITCKASQGINSFLTWFQQKPGKSPKTLIY |
| | | RANRLVDGVPSRFSGSGSGQDYSLTISSLEYEDVGIYYCLQYDEFPRTF |
| | | GGGTKLEIK |
| F1.245.2 | SEQ ID NO. 52 | DFQMTQTTSFLSASLGDRVTISCSASQDISNYLNWYQQKPDGTVKLLI |
| | | YYTSSLHSGVPSRFSGSGSGTDYSLTISNLEPEDIATYYCQQYSMLPYT |
| | | FGGGTKLEME |
| F1.60.9 | SEQ ID NO. 54 | DIKMTQSPSSMNASLGERVTITCKASQDINSYLSWFQQKPGKSPKILIY |
| | | RANRLIDGVPSRFSGSGSGQDYSLTISSLEYEDMGIYYCLQYGVFPLTF |
| | | GAGTKLELK |
| F1.172.13 | SEQ ID NO. 56 | DIQMTQSPASLSASVGETVTITCRASGNIHNSLAWYQQKQGKSPQLLV |
| | | HNAKTLADGVPSRFSGSGSGTQYSLKINSLQPEDFGSYYCQHFWSTPP |
| | | FGGGTKLEIK |
| F1.17.1 | SEQ ID NO. 58 | QIVLTQSPAILSTSPGEKVTMTCSASSRVNYMYWYQQKPGSSPRLLIY |
| | | DTSNLASGVPVRESGSGSGTSYSLTISRMEAEDAATYYCQQWTSYPLT |
| | | FGAGTKLELK |
| F2.151.13 | SEQ ID NO. 60 | QAVVTQESALTTSPGETVTLTCRSSTGAVTTSNYANWVQEKPDHLFT |
| | | GLIGGTNNQAPGVPARFSGSLIGDKAALTISGAQTEDEAIYFCALWFS |
| | | NHWVFGGGTKLTVL |
| F2.70.2 | SEQ ID NO. 62 | QLVLTQSSSASFSLGASAKLTCTLSSQHSSYIIEWYQQQPLKPPKYVME |
| | | LKKDGSHSTGDGIPDRFSGSSSGADRYLSISNIQPEDEAIYICGVGDTIK |
| | | EQFVYVFGGGTKVTVL |
| F2.104.10 | SEQ ID NO. 64 | QLVLTQSSSASFSLGASAKLTCTLSSQHSSYIIEWYQQQPLKPPKYVME |
| | | VKKDGSHSTGDGIPDRFSGSSSGADRYLSISNIQSEDEAIYICGVGDTIK |
| | | EQFVYVFGGGTKVTVL |
| F2.180.16 | SEQ ID NO. 66 | QPVLTQSSSASFSLGASAKLTCTLSSQYSTYTIGWYQQQPLKPPKYVM |
| | | ELKHDGSRHTGDGIPDRFSGSSSGADRYLSISNIQPEDEATYICGVGDTI |
| | | KEQFMYVFGGGTKVTVL |
| F2.121.9 | SEQ ID NO. 68 | QAVVTQESALTTSPGGTVVLTCRSNTGAVTTSNYANWVQEKPDHLFT |
| | | GLIGGTSNRAPGVPVRFSGSLIGDKAALTITGAQTEDDAMYFCALWYS |
| | | THWVFGGGTKLTVL |
| F2.173.9 | SEQ ID NO. 70 | QLVLTQSSSASFSLGASAKLTCTLSSQHSTYTIEWYQQQPHKPPKFVM |
| | | EIKKDGSHYTGDGIPDRFSGSSSGADRYLSISNIQPEDETIYICGVSDMI |
| | | KDQFVYVFGGGTKVTVL |
| F2.343.16 | SEQ ID NO. 72 | QAVVTQESALTTSPGETVTLTCRSSTGAVTTSNYANWVQEKPDHLFT |
| | | GLIGGTYNRVPGVPARFSGSLIGDKAALTITGAQTEDEAIYFCALWYS |
| | | NHFWVFGGGTKLTVL |
| F2.205.9 | SEQ ID NO. 74 | QAVVTQESALTTSPGETVTLTCRSSTGAVTTRNYANWVQEKPDHLFT |
| | | GLIGGTNNRAPGVPARFSGSLIGDKAALTITGAQTEDEAIYFCALWYS |
| | | NHFIFGSGTKVTVL |
| F2.99.1 | SEQ ID NO. 76 | QLVLTQSSSASFSLGASAKLTCTLSRQHSAYTIEWYQQQPLKPPKYVM |
| | | EVKKDGSHSTGDGIPDRFSGSSSGADRYLSISNIQPEDEAIYICGVGDTI |
| | | KEHFVFGGGTKVTVL |
| F2.127.11 | SEQ ID NO. 78 | QPVITQSSSASFSLGASAELTCTLSSQHSTYTIEWYQQQPLKPPKYVMG |
| | | LKKDGSHSTGDGIPDRFSGSSSGADRYLSISNIQPEDEAIYICGVSDTIK |
| | | EQFVYVFGGGTKVTVL |
| F2.55.1 | SEQ ID NO. 80 | QLVLTQSSSASFSLGASAKLTCTLSSQHSAYTVEWYQQQPLKPPKYV |
| | | MELKKDGSHSTGDGIPDRFSGSSSGADRYLSISNIQPEDEAIYICGVGD |
| | | TVKEQFVFGGGTKVTVL |
| F2.42.9 | SEQ ID NO. 82 | QLVLTQSSSASFSLGASAKLTCTLSSQHSAYTIEWYQQQPLKPPKFVM |
| | | DLKKDGSHSTGDGIPARFSGSSSGADRYLTISNIQPEDEAIYICGVGDTI |
| | | KEQYVFGGGTKVTVL |

[symbol]

indicates data missing or illegible when filed

TABLE 10
Specific sequence information of CDRs of CD22 antibody analyzed by
IMGT, KABAT and Chothia software
IMGT analysis

| Antibody | Sequence | | Sequence | | Sequence | |
|---|---|---|---|---|---|---|
| number | number | CDR1-HC | number | CDR2-HC | number | CDR3-HC |
| [symbol] | SEQ ID | [symbol] | SEQ ID | [symbol] | SEQ ID | [symbol] |
| | NO: 83 | | NO. 84 | | NO. 85 | |
| [symbol] | SEQ ID | [symbol] | SEQ ID | [symbol] | SEQ ID | [symbol] |
| | NO. 101 | | NO. 102 | | NO. 103 | |
| [symbol] | SEQ ID | [symbol] | SEQ ID | [symbol] | SEQ ID | [symbol] |
| | NO. 119 | | NO. 120 | | NO. 121 | |
| [symbol] | SEQ ID | [symbol] | SEQ ID | [symbol] | SEQ ID | [symbol] |
| | NO. 137 | | NO. 138 | | NO. 139 | [symbol] |
| [symbol] | SEQ ID | [symbol] | SEQ ID | [symbol] | SEQ ID | [symbol] |
| | NO. 155 | | NO. 156 | | NO. 157 | |
| [symbol] | SEQ ID | [symbol] | SEQ ID | [symbol] | SEQ ID | [symbol] |
| | NO. 173 | | NO. 174 | | NO. 175 | |
| [symbol] | SEQ ID | [symbol] | SEQ ID | [symbol] | SEQ ID | [symbol] |
| | NO. 191 | | NO. 192 | | NO. 193 | |
| F1.257.3 | SEQ ID | GYTFTSYG | SEQ ID | IYPKLGTT | SEQ ID | ACPHYYATR |
| | NO. 209 | | NO. 210 | | NO. 211 | GGDY |
| F1.105.11 | SEQ ID | GYTFSDYF | SEQ ID | INSYSGGT | SEQ ID | ARWMDY |
| | NO. 227 | | NO. 228 | | NO. 229 | |
| F1.267.9 | SEQ ID | GYTFTDFY | SEQ ID | INPYNGGI | SEQ ID | ARRMEYHA |
| | NO. 245 | | NO. 246 | | NO. 247 | MDY |
| F1.7.6 | SEQ ID | GYDFTRYN | SEQ ID | IDPYNGDT | SEQ ID | EAIYYDMEG |
| | NO. 263 | | NO. 264 | | NO. 265 | YALDY |
| F1.224.1 | SEQ ID | GYTFTDFN | SEQ ID | VNPNNGGT | SEQ ID | VRLGTSDYG |
| | NO. 281 | | NO. 282 | | NO. 283 | EAWFIS |
| F1.250.16 | SEQ ID | GYTFTTYP | SEQ ID | IHPYNDDT | SEQ ID | VRGDYYNSR |
| | NO. 299 | | NO. 300 | | NO. 301 | YWYFDV |
| F1.120.15 | SEQ ID | GNAFSNSW | SEQ ID | VYSEDGDT | SEQ ID | ARWLIYYGT |
| | NO. 317 | | NO. 318 | | NO. 319 | YGAMDY |
| F1.62.10 | SEQ ID | GYEFSSIW | SEQ ID | IFPGDGEI | SEQ ID | AGWRIYYGT |
| | NO. 335 | | NO. 336 | | NO. 337 | YGAMDY |
| F1.216.2 | SEQ ID | GYAFSSSW | SEQ ID | IYPGDGDT | SEQ ID | AREGSYYSNP |
| | NO. 353 | | NO. 354 | | NO. 355 | WYFDY |
| F1.280.1 | SEQ ID | GYTFTTYG | SEQ ID | INTDSGVP | SEQ ID | ARGGPDY |
| | NO. 371 | | NO. 372 | | NO. 373 | |
| F1.200.11 | SEQ ID | GYTFTTAG | SEQ ID | INTLSGEP | SEQ ID | ARAYYSNLY |
| | NO. 389 | | NO. 390 | | NO. 391 | WYFDV |
| F1.192.1 | SEQ ID | DFNIKEDY | SEQ ID | IDPENGDT | SEQ ID | ATYGSLDY |
| | NO. 407 | | NO. 408 | | NO. 409 | |
| F1.245.2 | SEQ ID | GFNIKDYY | SEQ ID | IDPEDGET | SEQ ID | AREAAGYFY |
| | NO. 425 | | NO. 426 | | NO. 427 | RGSSYGYFD |
| | | | | | | V |
| F1.60.9 | SEQ ID | GLSLNNYG | SEQ ID | IWGDGST | SEQ ID | AINWGDY |
| | NO. 443 | | NO. 444 | | NO. 445 | |
| F1.172.13 | SEQ ID | GFSLSTSGM | SEQ ID | IYWDDDK | SEQ ID | ARAPPPNWD |
| | NO. 461 | G | NO. 462 | | NO. 463 | EYYFDY |
| F1.17.1 | SEQ ID | GFSLSRYT | SEQ ID | IWGGGST | SEQ ID | ARPHDFDAG |
| | NO. 479 | | NO. 480 | | NO. 481 | GFAY |
| F2.151.13 | SEQ ID | GYTFRDYW | SEQ ID | ILPGSGNA | SEQ ID | ARVYSNWYF |
| | NO. 497 | | NO. 498 | | NO. 499 | DA |
| F2.70.2 | SEQ ID | GFTFSDYG | SEQ ID | ISRGSHTI | SEQ ID | VRMAGYYA |
| | NO. 515 | | NO. 516 | | NO. 517 | MDY |
| F2.104.10 | SEQ ID | GFTFSDYG | SEQ ID | ISRGSHTI | SEQ ID | VRMAGYYA |
| | NO. 533 | | NO. 534 | | NO. 535 | MDY |
| F2.180.16 | SEQ ID | GFTFSDYY | SEQ ID | ISNTGYST | SEQ ID | ARHGILYAM |
| | NO. 551 | | NO. 552 | | NO. 553 | DY |
| F2.121.9 | SEQ ID | GFTFSDYV | SEQ ID | ISDAGSYT | SEQ ID | AIIYFGNYGG |
| | NO. 569 | | NO. 570 | | NO. 571 | Y |
| F2.173.9 | SEQ ID | GFTFSNYA | SEQ ID | ISSIGSFT | SEQ ID | ARLGVYFDS |
| | NO. 587 | | NO. 588 | | NO. 589 | |
| F2.343.16 | SEQ ID | GFSLTGYG | SEQ ID | IWGGGGT | SEQ ID | ARYSKYGHF |
| | NO. 605 | | NO. 606 | | NO. 607 | DV |
| F2.205.9 | SEQ ID | GFSLSTSGM | SEQ ID | IWWDDDE | SEQ ID | ARISIPYGYY |
| | NO. 623 | | NO. 624 | | NO. 623 | DWFFDV |
| [symbol] | SEQ ID | [symbol] | SEQ ID | [symbol] | SEQID | [symbol] |
| | NO. 641 | [symbol] | NO. 642 | | NO. 643 | [symbol] |
| [symbol] | SEQ ID | [symbol] | SEQ ID | [symbol] | SEQ ID | [symbol] |
| | NO. 659 | [symbol] | NO. 660 | | NO. 661 | [symbol] |
| [symbol] | SEQ ID | [symbol] | SEQ ID | [symbol] | SEQ ID | [symbol] |
| | NO. 677 | [symbol] | NO. 678 | | NO. 679 | [symbol] |
| [symbol] | SEQ ID | [symbol] | SEQ ID | [symbol] | SEQ ID | [symbol] |
| | NO. 695 | [symbol] | NO. 696 | | NO. 697 | [symbol] |
| Antibody | Sequence | | Sequence | | Sequence | |
| number | number | CDR1-LC | number | CDR2-LC | number | CDR3-LC |
| [symbol] | SEQ ID | [symbol] | SEQ ID | RA | SEQ ID | [symbol] |
| | NO. 92 | | NO. 93 | | NO. 94 | |
| [symbol] | SEQ ID | [symbol] | SEQ ID | RA | SEQ ID | [symbol] |
| | NO. 110 | | NO. 111 | | NO. 112 | |
| [symbol] | SEQ ID | [symbol] | SEQ ID | RA | SEQ ID | [symbol] |
| | NO. 128 | | NO. 129 | | NO. 130 | |
| [symbol] | SEQ ID | [symbol] | SEQ ID | TV | SEQ ID | [symbol] |
| | NO. 146 | [symbol] | NO. 147 | | NO. 148 | |
| [symbol] | SEQ ID | [symbol] | SEQ ID | RT | SEQ ID | [symbol] |
| | NO. 164 | | NO. 165 | | NO. 166 | |
| [symbol] | SEQ ID | [symbol] | SEQ ID | RA | SEQ ID | [symbol] |
| | NO. 182 | | NO. 183 | | NO. 184 | |
| [symbol] | SEQ ID | [symbol] | SEQ ID | [symbol] | SEQ ID | [symbol] |
| | NO. 200 | | NO. 201 | | NO. 202 | |
| [symbol] | SEQ ID | [symbol] | SEQ ID | YA | SEQ ID | [symbol] |
| | NO. 218 | | NO. 219 | | NO. 220 | |
| [symbol] | SEQ ID | [symbol] | SEQ ID | SG | SEQ ID | [symbol] |
| | NO. 236 | | NO. 237 | | NO. 238 | |
| [symbol] | SEQ ID | [symbol] | SEQ ID | RA | SEQ ID | [symbol] |
| | NO. 254 | [symbol] | NO. 255 | | NO. 256 | |
| [symbol] | SEQ ID | [symbol] | SEQ ID | WA | SEQ ID | [symbol] |
| | NO. 272 | [symbol] | NO. 273 | | NO. 274 | |
| [symbol] | SEQ ID | [symbol] | SEQ ID | YA | SEQ ID | [symbol] |
| | NO. 290 | | NO. 291 | | NO. 292 | |
| [symbol] | SEQ ID | [symbol] | SEQ ID | SA | SEQ ID | [symbol] |
| | NO. 308 | | NO. 309 | | NO. 310 | |
| [symbol] | SEQ ID | [symbol] | SEQ ID | FT | SEQ ID | [symbol] |
| | NO. 326 | | NO. 327 | | NO. 328 | |
| [symbol] | SEQ ID | [symbol] | SEQ ID | YT | SEQ ID | [symbol] |
| | NO. 344 | | NO. 345 | | NO. 346 | |
| [symbol] | SEQ ID | [symbol] | SEQ ID | KV | SEQ ID | [symbol] |
| | NO. 362 | [symbol] | NO. 363 | | NO. 364 | |
| [symbol] | SEQ ID | [symbol] | SEQ ID | YA | SEQ ID | [symbol] |
| | NO. 380 | | NO. 381 | | NO. 382 | |
| [symbol] | SEQ ID | [symbol] | SEQ ID | SA | SEQ ID | [symbol] |
| | NO. 398 | | NO. 399 | | NO. 400 | |
| [symbol] | SEQ ID | [symbol] | SEQ ID | RA | SEQ ID | [symbol] |
| | NO. 416 | | NO. 417 | | NO. 418 | |
| [symbol] | SEQ ID | [symbol] | SEQ ID | YT | SEQ ID | [symbol] |
| | NO. 434 | | NO. 435 | | NO. 436 | |
| [symbol] | SEQ ID | [symbol] | SEQ ID | RA | SEQ ID | [symbol] |
| | NO. 452 | | NO. 453 | | NO. 454 | |
| [symbol] | SEQ ID | [symbol] | SEQ ID | NA | SEQ ID | [symbol] |
| | NO. 470 | | NO. 471 | | NO. 472 | |
| [symbol] | SEQ ID | [symbol] | SEQ ID | DT | SEQ ID | [symbol] |
| | NO. 488 | | NO. 489 | | NO. 490 | |
| [symbol] | SEQ ID | [symbol] | SEQ ID | GT | SEQ ID | [symbol] |
| | NO. 506 | [symbol] | NO. 507 | | NO. 508 | |
| [symbol] | SEQ ID | [symbol] | SEQ ID | [symbol] | SEQ ID | [symbol] |
| | NO. 524 | | NO. 525 | | NO. 526 | [symbol] |
| [symbol] | SEQ ID | [symbol] | SEQ ID | [symbol] | SEQ ID | [symbol] |
| | NO. 542 | | NO. 543 | | NO. 544 | [symbol] |
| [symbol] | SEQ ID | [symbol] | SEQ ID | [symbol] | SEQ ID | [symbol] |
| | NO. 560 | | NO. 561 | | NO. 562 | |
| [symbol] | SEQ ID | [symbol] | SEQ ID | GT | SEQ ID | [symbol] |
| | NO. 578 | | NO. 579 | | NO. 580 | |
| [symbol] | SEQ ID | [symbol] | SEQ ID | [symbol] | SEO ID | [symbol] |
| | NO. 596 | | NO. 597 | | NO. 598 | [symbol] |
| [symbol] | SEQ ID | [symbol] | SEQ ID | GT | SEQ ID | [symbol] |
| | NO. 614 | | NO. 615 | | NO. 616 | |
| [symbol] | SEQ ID | [symbol] | SEQ ID | GT | SEQ ID | [symbol] |
| | NO. 632 | | NO. 633 | | NO. 634 | |
| [symbol] | SEQ ID | [symbol] | SEQ ID | [symbol] | SEQ ID | [symbol] |
| | NO. 650 | | NO. 651 | | NO. 652 | |
| [symbol] | SEQ ID | [symbol] | SEQ ID | [symbol] | SEQ ID | [symbol] |
| | NO. 668 | | NO. 669 | | NO. 670 | |
| [symbol] | SEQ ID | [symbol] | SEQ ID | [symbol] | SEQ ID | [symbol] |
| | NO. 686 | | NO. 687 | | NO. 688 | [symbol] |
| [symbol] | SEO ID | [symbol] | SEQ ID | [symbol] | SEQ ID | [symbol] |
| | NO. 704 | | NO. 705 | | NO. 706 | [symbol] |

KABAT analysis

| Antibody | Sequence | | Sequence | | Sequence | |
|---|---|---|---|---|---|---|
| number | number | CDR1-HC | number | CDR2-HC | number | CDR3-HC |
| [symbol] | SEQ ID | [symbol] | SEQ ID | [symbol] | SEQ ID | [symbol] |
| | NO. 86 | | NO. 87 | [symbol] | NO. 88 | |
| [symbol] | SEQ ID | [symbol] | SEQ ID | [symbol] | SEQ ID | [symbol] |
| | NO. 104 | | NO. 105 | [symbol] | NO. 106 | |
| [symbol] | SEQ ID | [symbol] | SEQ ID | [symbol] | SEQ ID | [symbol] |
| | NO. 122 | | NO. 123 | [symbol] | NO. 124 | |
| [symbol] | SEQ ID | [symbol] | SEQ ID | [symbol] | SEQ ID | [symbol] |
| | NO. 146 | | NO. 147 | | NO. 148 | |
| [symbol] | SEQ ID | [symbol] | SEQ ID | [symbol] | SEQ ID | [symbol] |
| | NO. 158 | | NO. 159 | [symbol] | NO. 160 | |
| [symbol] | SEQ ID | [symbol] | SEQ ID | [symbol] | SEQ ID | [symbol] |
| | NO. 176 | | NO. 177 | [symbol] | NO. 178 | |
| F1.161.7 | SEQ ID | SYWIS | SEQ ID | DIYPGTGSSNYN | SEQ ID | LKFEGIGY |
| | NO. 194 | | NO. 195 | EKFKS | NO. 196 | |
| F1.257.3 | SEQ ID | SYGIS | SEQ ID | EIYPKLGTTYYN | SEQ ID | PHYYATRGG |
| | NO. 212 | | NO. 213 | EKFKD | NO. 214 | DY |
| F1.105.11 | SEQ ID | DYFMN | SEQ ID | IINSYSGGTSYNQ | SEQ ID | WMDY |
| | NO. 230 | | NO. 231 | KFKG | NO. 232 | |
| F1.267.9 | SEQ ID | DFYMN | SEQ ID | VINPYNGGINYN | SEQ ID | RMEYHAMD |
| | NO. 248 | | NO. 249 | QKFKG | NO. 250 | Y |
| F1.7.6 | SEQ ID | RYNMY | SEQ ID | YIDPYNGDTRYN | SEQ ID | IYYDMEGYA |
| | NO. 266 | | NO. 267 | QKFKG | NO. 268 | LDY |
| F1.224.1 | SEQ ID | DFNMD | SEQ ID | DVNPNNGGTIYN | SEQ ID | LGTSDYGEA |
| | NO. 284 | | NO. 285 | QKFKG | NO. 286 | WFIS |
| F1.250.16 | SEQ ID | TYPIE | SEQ ID | NIHPYNDDTEYN | SEQ ID | GDYYNSRYW |
| | NO. 302 | | NO. 303 | EKFKG | NO. 304 | YFDV |
| F1.120.15 | SEQ ID | NSWMN | SEQ ID | RVYSEDGDTQY | SEQ ID | WLIYYGTYG |
| | NO. 320 | | NO. 321 | NGKFRD | NO. 322 | AMDY |
| F1.62.10 | SEQ ID | SIWMN | SEQ ID | RIFPGDGEINYNE | SEQ ID | WRIYYGTYG |
| | NO. 338 | | NO. 339 | KFKG | NO. 340 | AMDY |
| F1.216.2 | SEQ ID | SSWMN | SEQ ID | RIYPGDGDTNYS | SEQ ID | EGSYYSNPW |
| | NO. 356 | | NO. 357 | GEFKV | NO. 358 | YFDY |
| F1.280.1 | SEQ ID | TYGMS | SEQ ID | WINTDSGVPTYA | SEQ ID | GGPDY |
| | NO. 374 | | NO. 375 | DDFKR | NO. 376 | |
| F1.200.11 | SEQ ID | TAGMQ | SEQ ID | WINTLSGEPKYA | SEQ ID | AYYSNLYWY |
| | NO. 392 | | NO. 393 | EDFKG | NO. 394 | FDV |
| F1.192.1 | SEQ ID | EDYIH | SEQ ID | WIDPENGDTEYA | SEQ ID | YGSLDY |
| | NO. 410 | | NO. 411 | SKFQG | NO. 412 | |
| F1.245.2 | SEQ ID | DYYIH | SEQ ID | RIDPEDGETEYAP | SEQ ID | EAAGYFYRG |
| | NO. 428 | | NO. 429 | KFQD | NO. 430 | SSYGYFDV |
| F1.60.9 | SEQ ID | NYGVS | SEQ ID | VIWGDGSTNYHS | SEQ ID | NWGDY |
| | NO. 446 | | NO. 447 | ALIS | NO. 448 | |
| F1.172.13 | SEQ ID | TSGMGVS | SEQ ID | HIYWDDDKRYN | SEQ ID | APPPNWDEY |
| | NO. 464 | | NO. 465 | PSLKS | NO. 466 | YFDY |
| F1.17.1 | SEQ ID | RYTVH | SEQ ID | MIWGGGSTDYN | SEQ ID | PHDFDAGGF |
| | NO. 482 | | NO. 483 | SALKS | NO. 484 | AY |
| F2.151.13 | SEQ ID | DYWLE | SEQ ID | EILPGSGNAYYN | SEQ ID | VYSNWYFDA |
| | NO. 500 | | NO. 501 | EKFKG | NO. 502 | |
| F2.70.2 | SEQ ID | DYGMN | SEQ ID | YISRGSHTIYYAD | SEQ ID | MAGYYAMD |
| | NO. 518 | | NO. 519 | TVKG | NO. 520 | Y |
| F2.104.10 | SEQ ID | DYGMN | SEQ ID | YISRGSHTIYYAD | SEQ ID | MAGYYAMD |
| | NO. 536 | | NO. 537 | TVKG | NO. 538 | Y |
| F2.180.16 | SEQ ID | DYYMY | SEQ ID | YISNTGYSTFYPD | SEQ ID | HGILYAMDY |
| | NO. 554 | | NO. 555 | SVKG | NO. 556 | |
| F2.121.9 | SEQ ID | DYVMS | SEQ ID | TISDAGSYTFYPD | SEQ ID | IYFGNYGGY |
| | NO. 572 | | NO. 573 | NLKG | NO. 574 | |
| F2.173.9 | SEQ ID | NYAMS | SEQ ID | TISSIGSFTYYSDS | SEQ ID | LGVYFDS |
| | NO. 590 | | NO. 591 | VKG | NO. 592 | |
| F2.343.16 | SEQ ID | GYGVQ | SEQ ID | VIWGGGGTDYN | SEQ ID | YSKYGHFDV |
| | NO. 608 | | NO. 609 | AAFIS | NO. 610 | |
| [symbol] | SEQ ID | [symbol] | SEQ ID | [symbol] | SEQ ID | [symbol] |
| | NO. 626 | | NO. 627 | [symbol] | NO. 628 | [symbol] |
| [symbol] | SEQ ID | [symbol] | SEQ ID | [symbol] | SEQ ID | [symbol] |
| | NO. 644 | | NO. 645 | [symbol] | NO. 646 | [symbol] |
| [symbol] | SEQ ID | [symbol] | SEQ ID | [symbol] | SEQ ID | [symbol] |
| | NO. 662 | | NO. 663 | [symbol] | NO. 664 | |
| [symbol] | SEQ ID | [symbol] | SEQ ID | [symbol] | SEQ ID | [symbol] |
| | NO. 680 | | NO. 681 | | NO. 682 | [symbol] |
| [symbol] | SEQ ID | [symbol] | SEQ ID | [symbol] | SEQ ID | [symbol] |
| | NO. 698 | | NO. 699 | [symbol] | NO. 700 | [symbol] |
| Antibody | Sequence | | Sequence | | Sequence | |
| number | number | CDR1-LC | number | CDR2-LC | nuraber | CDR3-LC |
| [symbol] | SEQ ID | [symbol] | SEQ ID | [symbol] | SEQ ID | [symbol] |
| | NO. 95 | [symbol] | NO. 96 | | NO. 97 | |
| [symbol] | SEQ ID | [symbol] | SEQ ID | [symbol] | SEQ ID | [symbol] |
| | NO. 113 | [symbol] | NO. 114 | | NO. 115 | |
| [symbol] | SEQ ID | [symbol] | SEQ ID | [symbol] | SEQ ID | [symbol] |
| | NO. 131 | [symbol] | NO. 132 | | NO. 133 | |
| [symbol] | SEQ ID | [symbol] | SEQ ID | [symbol] | SEQ ID | [symbol] |
| | NO. 149 | [symbol] | NO. 150 | | NO. 151 | |
| [symbol] | SEQ ID | [symbol] | SEQ ID | [symbol] | SEQ ID | [symbol] |
| | NO. 167 | [symbol] | NO. 168 | | NO. 169 | |
| [symbol] | SEQ ID | [symbol] | SEQ ID | [symbol] | SEQ ID | [symbol] |
| | NO. 185 | [symbol] | NO. 186 | | NO. 187 | |
| [symbol] | SEQ ID | [symbol] | SEQ ID | [symbol] | SEQ ID | [symbol] |
| | NO. 203 | | NO. 204 | | NO. 205 | |
| [symbol] | SEQ ID | [symbol] | SEQ ID | [symbol] | SEQ ID | [symbol] |
| | NO. 221 | [symbol] | NO. 222 | | NO. 223 | |
| [symbol] | SEQ ID | [symbol] | SEQ ID | [symbol] | SEQ ID | [symbol] |
| | NO. 239 | [symbol] | NO. 240 | | NO: 241 | |
| [symbol] | SEQ ID | [symbol] | SEQ ID | [symbol] | SEQ ID | [symbol] |
| | NO. 257 | [symbol] | NO. 258 | | NO. 259 | |
| [symbol] | SEQ ID | [symbol] | SEQ ID | [symbol] | SEQ ID | [symbol] |
| | NO. 275 | [symbol] | NO. 276 | | NO. 277 | |
| [symbol] | SEQ ID | [symbol] | SEQ ID | [symbol] | SEQ ID | [symbol] |
| | NO. 293 | [symbol] | NO. 294 | | NO. 295 | |
| [symbol] | SEQ ID | [symbol] | SEQ ID | [symbol] | SEQ ID | [symbol] |
| | NO. 311 | [symbol] | NO. 312 | | NO. 313 | |
| [symbol] | SEQ ID | [symbol] | SEQ ID | [symbol] | SEQ ID | [symbol] |
| | NO. 329 | [symbol] | NO. 330 | | NO. 331 | |
| [symbol] | SEQ ID | [symbol] | SEQ ID | [symbol] | SEQ ID | [symbol] |
| | NO. 347 | [symbol] | NO. 348 | | NO. 349 | |
| [symbol] | SEQ ID | [symbol] | SEQ ID | [symbol] | SEQ ID | [symbol] |
| | NO. 365 | [symbol] | NO. 366 | | NO. 367 | |
| [symbol] | SEQ ID | [symbol] | SEQ ID | [symbol] | SEQ ID | [symbol] |
| | NO. 383 | [symbol] | NO. 384 | | NO. 385 | |
| [symbol] | SEQ ID | [symbol] | SEQ ID | [symbol] | SEQ ID | [symbol] |
| | NO. 401 | [symbol] | NO. 402 | | NO. 403 | |
| [symbol] | SEQ ID | [symbol] | SEQ ID | [symbol] | SEQ ID | [symbol] |
| | NO. 419 | | NO. 420 | | NO. 421 | |
| [symbol] | SEQ ID | [symbol] | SEQ ID | [symbol] | SEQ ID | [symbol] |
| | NO. 437 | | NO. 438 | | NO. 439 | |
| [symbol] | SEQ ID | [symbol] | SEQ ID | [symbol] | SEQ ID | [symbol] |
| | NO. 453 | [symbol] | NO. 456 | | NO. 457 | |
| [symbol] | SEQ ID | [symbol] | SEQ ID | [symbol] | SEQ ID | [symbol] |
| | NO. 473 | | NO. 474 | | NO. 475 | |
| [symbol] | SEQ ID | [symbol] | SEQ ID | [symbol] | SEQ ID | [symbol] |
| | NO. 491 | [symbol] | NO. 492 | | NO. 493 | |
| [symbol] | SEQ ID: | [symbol] | SEQ ID | [symbol] | SEQ ID | [symbol] |
| | NO. 509 | [symbol] | NO. 510 | | NO. 511 | |
| [symbol] | SEQ ID | [symbol] | SEQ ID | [symbol] | SEQ ID | [symbol] |
| | NO. 527 | | NO. 528 | | NO. 529 | [symbol] |
| [symbol] | SEQ ID | [symbol] | SEQ ID | [symbol] | SEQ ID | [symbol] |
| | NO. 545 | | NO. 546 | | NO. 547 | [symbol] |
| [symbol] | SEQ ID | [symbol] | SEQ ID | [symbol] | SEQ ID | [symbol] |
| | NO. 563 | [symbol] | NO. 564 | | NO. 565 | [symbol] |
| [symbol] | SEQ ID | [symbol] | SEQ ID | [symbol] | SEQ ID | [symbol] |
| | NO. 581 | [symbol] | NO. 582 | | NO. 583 | |
| [symbol] | SEQ ID | [symbol] | SEQ ID | [symbol] | SEQ ID | [symbol] |
| | NO. 599 | [symbol] | NO. 600 | | NO. 601 | [symbol] |
| [symbol] | SEQ ID | [symbol] | SEQ ID | [symbol] | SEQ ID | [symbol] |
| | NO. 617 | [symbol] | NO. 618 | | NO. 619 | [symbol] |
| [symbol] | SEQ ID | [symbol] | SEQ ID | [symbol] | SEQ ID | [symbol] |
| | NO. 635 | [symbol] | NO. 636 | | NO. 637 | |
| [symbol] | SEQ ID | [symbol] | SEQ ID | [symbol] | SEQ ID | [symbol] |
| | NO. 653 | [symbol] | NO. 654 | | NO. 655 | [symbol] |
| [symbol] | SEQ ID | [symbol] | SEQ ID | [symbol] | SEQ ID | [symbol] |
| | NO. 671 | [symbol] | NO. 672 | | NO. 673 | [symbol] |
| [symbol] | SEQ ID | [symbol] | SEQ ID | [symbol] | SEQ ID | [symbol] |
| | NO. 689 | | NO. 690 | | NO. 691 | [symbol] |
| [symbol] | SEQ ID | [symbol] | SEQ ID | [symbol] | SEQ ID | [symbol] |
| | NO. 707 | [symbol] | NO. 708 | | NO.709 | [symbol] |

Chothia analysis

| Antibody | Sequence | | Sequence | | Sequence | |
|---|---|---|---|---|---|---|
| number | number | CDR1-HC | number | CDR2-HC | number | CDR3-HC |
| [symbol] | SEQ ID | [symbol] | SEQ ID | [symbol] | SEQ ID | [symbol] |
| | NO. 89 | | NO. 90 | | NO. 91 | |
| [symbol] | SEQ ID | [symbol] | SEQ ID | [symbol] | SEQ ID | [symbol] |
| | NO. 107 | | NO. 108 | | NO. 109 | |
| [symbol] | SEQ ID | [symbol] | SEQ ID | [symbol] | SEQ ID | [symbol] |
| | NO. 125 | | NO. 126 | | NO. 127 | |
| [symbol] | SEQ ID | [symbol] | SEQ ID | [symbol] | SEQ ID | [symbol] |
| | NO. 143 | | NO. 144 | | NO. 145 | [symbol] |
| [symbol] | SEQ ID | [symbol] | SEQ ID | [symbol] | SEQ ID | [symbol] |
| | NO. 161 | | NO. 162 | | NO. 163 | |
| F1.77.9 | SEQ ID | GYTFTTY | SEQ ID | NPSSGY | SEQ ID | QLDY |
| | NO. 179 | | NO. 180 | | NO. 181 | |
| F1.161.7 | SEQ ID | GYTFTSY | SEQ ID | YPGTGS | SEQ ID | LKFEGIGY |
| | NO. 197 | | NO. 198 | | NO. 199 | |
| F1.257.3 | SEQ ID | GYTFTSY | SEQ ID | YPKLGT | SEQ ID | PHYYATRGG |
| | NO. 215 | | NO. 216 | | NO. 217 | DY |
| F1.105.11 | SEQ ID | GYTFSDY | SEQ ID | NSYSGG | SEQ ID | WMDY |
| | NO. 233 | | NO. 234 | | NO. 235 | |
| F1.267.9 | SEQ ID | GYTFTDF | SEQ ID | NPYNGG | SEQ ID | RMEYHAMD |
| | NO. 251 | | NO. 252 | | NO. 253 | Y |
| F1.7.6 | SEQ ID | GYDFTRY | SEQ ID | DPYNGD | SEQ ID | IYYDMEGYA |
| | NO. 269 | | NO. 270 | | NO. 271 | LDY |
| F1.224.1 | SEQ ID | GYTFTDF | SEQ ID | NPNNGG | SEQ ID | LGTSDYGEA |
| | NO. 287 | | NO. 288 | | NO. 289 | WFIS |
| F1.250.16 | SEQ ID | GYTFTTY | SEQ ID | HPYNDD | SEQ ID | GDYYNSRYW |
| | NO. 305 | | NO. 306 | | NO. 307 | YFDV |
| F1.120.15 | SEQ ID | GNAFSNS | SEQ ID | YSEDGD | SEQ ID | WLIYYGTYG |
| | NO. 323 | | NO. 324 | | NO. 325 | AMDY |
| F1.62.10 | SEQ ID | GYEFSSI | SEQ ID | FPGDGE | SEQ ID | WRIYYGTYG |
| | NO. 341 | | NO. 342 | | NO. 343 | AMDY |
| F1.216.2 | SEQ ID | GYAFSSS | SEQ ID | YPGDGD | SEQ ID | EGSYYSNPW |
| | NO. 359 | | NO. 360 | | NO. 361 | YFDY |
| F1.280.1 | SEQ ID | GYTFTTY | SEQ ID | NTDSGV | SEQ ID | GGPDY |
| | NO. 377 | | NO. 378 | | NO. 379 | |
| F1.200.11 | SEQ ID | GYTFTTA | SEQ ID | NTLSGE | SEQ ID | AYYSNLYWY |
| | NO. 395 | | NO. 396 | | NO. 397 | FDV |
| F1.192.1 | SEQ ID | DFNIKED | SEQ ID | DPENGD | SEQ ID | YGSLDY |
| | NO. 413 | | NO. 414 | | NO. 415 | |
| F1.245.2 | SEQ ID | GFNIKDY | SEQ ID | DPEDGE | SEQ ID | EAAGYFYRG |
| | NO. 431 | | NO. 432 | | NO. 433 | SSYGYFDV |
| F1.60.9 | SEQ ID | GLSLNNY | SEQ ID | WGDGS | SEQ ID | NWGDY |
| | NO. 449 | | NO. 450 | | NO. 451 | |
| F1.172.13 | SEQ ID | GFSLSTSGM | SEQ ID | YWDDD | SEQ ID | APPPNWDEY |
| | NO. 467 | | NO. 468 | | NO. 469 | YFDY |
| F1.17.1 | SEQ ID | GFSLSRY | SEQ ID | WGGGS | SEQ ID | PHDFDAGGF |
| | NO. 485 | | NO. 486 | | NO. 487 | AY |
| F2.151.13 | SEQ ID | GYTFRDY | SEQ ID | LPGSGN | SEQ ID | VYSNWYFDA |
| | NO. 503 | | NO. 504 | | NO. 505 | |
| F2.70.2 | SEQ ID | GFTFSDY | SEQ ID | SRGSHT | SEQ ID | MAGYYAMD |
| | NO. 521 | | NO. 522 | | NO. 523 | Y |
| F2.104.10 | SEQ ID | GFTFSDY | SEQ ID | SRGSHT | SEQ ID | MAGYYAMD |
| | NO. 539 | | NO. 540 | | NO. 541 | Y |
| F2.180.16 | SEQ ID | GFTFSDY | SEQ ID | SNTGYS | SEQ ID | HGILYAMDY |
| | NO. 557 | | NO. 558 | | NO. 559 | |
| F2.121.9 | SEQ ID | GFTESDY | SEQ ID | SDAGSY | SEQ ID | TYFGNYGGY |
| | NO. 575 | | NO. 576 | | NO. 577 | |
| F2.173.9 | SEQ ID | GFTFSNY | SEQ ID | SSIGSF | SEQ ID | LGVYFDS |
| | NO. 593 | | NO. 594 | | NO. 595 | |
| F2.343.16 | SEQ ID | GFSLTGY | SEQ ID | WGGGG | SEQ ID | YSKYGHFDV |
| | NO. 611 | | NO. 612 | | NO. 613 | |
| [symbol] | SEQ ID | [symbol] | SEQ ID | [symbol] | SEQ ID | [symbol] |
| | NO. 629 | | NO. 630 | | NO. 631 | [symbol] |
| [symbol] | SEQ ID | [symbol] | SEQ ID | [symbol] | SEQ ID | [symbol] |
| | NO. 647 | | NO. 648 | | NO. 649 | [symbol] |
| [symbol] | SEQ ID | [symbol] | SEQ ID | [symbol] | SEQ ID | [symbol] |
| | NO. 665 | | NO. 666 | | NO. 667 | |
| [symbol] | SEQ ID | [symbol] | SEQ ID | [symbol] | SEQ ID | [symbol] |
| | NO. 683 | | NO. 684 | | NO. 685 | [symbol] |
| [symbol] | SEQ ID | [symbol] | SEQ ID | [symbol] | SEQ ID | [symbol] |
| | NO: 701 | | NO. 702 | | NO. 703 | [symbol] |
| Antibody | Sequence | | Sequence | | Sequence | |
| number | number | CDR1-LC | number | CDR2-LC | number | CDR3-LC |
| [symbol] | SEQ ID | [symbol] | SEQ ID | [symbol] | SEQ ID | [symbol] |
| | NO. 98 | [symbol] | NO. 99 | | NO 100 | |
| [symbol] | SEQ ID | [symbol] | SEQ ID | [symbol] | SEQ ID | [symbol] |
| | NO. 116 | [symbol] | NO. 117 | | NO. 118 | |
| [symbol] | SEQ ID | [symbol] | SEQ ID | [symbol] | SEQ ID | [symbol] |
| | NO. 134 | | NO. 135 | | NO. 135 | |
| [symbol] | SEQ ID | [symbol] | SEQ ID | [symbol] | SEQ ID | [symbol] |
| | NO. 152 | [symbol] | NO. 153 | | NO. 154 | |
| [symbol] | SEQ ID | [symbol] | SEQ ID | [symbol] | SEQ ID | [symbol] |
| | NO. 170 | [symbol] | NO. 171 | | NO. 172 | |
| [symbol] | SEQ ID | [symbol] | SEQ ID | [symbol] | SEQ ID | [symbol] |
| | NO. 188 | [symbol] | NO. 189 | | NO. 190 | |
| [symbol] | SEQ ID | [symbol] | SEQ ID | [symbol] | SEQ ID | [symbol] |
| | NO. 206 | [symbol] | NO. 207 | | NO. 208 | |
| [symbol] | SEQ ID | [symbol] | SEQ ID | [symbol] | SEQ ID | [symbol] |
| | NO. 224 | | NO. 225 | | NO. 226 | |
| [symbol] | SEQ ID | [symbol] | SEQ ID | [symbol] | SEQ ID | [symbol] |
| | NO. 242 | [symbol] | NO. 243 | | NO. 244 | |
| [symbol] | SEQ ID | [symbol] | SEQ ID | [symbol] | SEQ ID | [symbol] |
| | NO. 260 | [symbol] | NO. 261 | | NO. 262 | |
| [symbol] | SEQ ID | [symbol] | SEQ ID | [symbol] | SEQ ID | [symbol] |
| | NO. 278 | [symbol] | NO. 279 | | NO. 280 | |
| [symbol] | SEQ ID | [symbol] | SEQ ID | [symbol] | SEQ ID | [symbol] |
| | NO. 296 | [symbol] | NO. 297 | | NO. 298 | |
| [symbol] | SEQ ID | [symbol] | SEQ ID | [symbol] | SEQ ID | [symbol] |
| | NO. 314 | [symbol] | NO. 315 | | NO. 316 | |
| [symbol] | SEQ ID | [symbol] | SEQ ID | [symbol] | SEQ ID | [symbol] |
| | NO. 332 | | NO. 333 | | NO. 334 | |
| [symbol] | SEQ ID | [symbol] | SEQ ID | [symbol] | SEQ ID | [symbol] |
| | NO. 350 | [symbol] | NO. 351 | | NO. 352 | |
| [symbol] | SEQ ID | [symbol] | SEQ ID | [symbol] | SEQ ID | [symbol] |
| | NO. 368 | [symbol] | NO. 369 | | NO. 370 | |
| [symbol] | SEQ ID | [symbol] | SEQ ID | [symbol] | SEQ ID | [symbol] |
| | NO. 386 | | NO. 387 | | NO. 388 | |
| [symbol] | SEQ ID | [symbol] | SEQ ID | [symbol] | SEQ ID | [symbol] |
| | NO. 404 | AVA | NO. 405 | | NO. 406 | |
| F1.192.1 | SEQ ID | KASQGINSF | SEQ ID | RANRLVD | SEQ ID | LQYDEFPRT |
| | NO. 422 | LT | NO. 423 | | NO. 424 | |
| F1.245.2 | SEQ ID | SASQDISNY | SEQ ID | YTSSLHS | SEQ ID | QQYSMLPYT |
| | NO. 440 | LN | NO. 441 | | NO. 442 | |
| F1.60.9 | SEQ ID | KASQDINSY | SEQ ID | RANRLID | SEQ ID | LQYGVFPLT |
| | NO. 458 | LS | NO. 459 | | NO. 460 | |
| F1.172.13 | SEQ ID | RASGNIHNS | SEQ ID | NAKTLAD | SEQ ID | QHFWSTPP |
| | NO. 476 | LA | NO. 477 | | NO. 478 | |
| F1.17.1 | SEQ ID | SASSRVNY | SEQ ID | DTSNLAS | SEQ ID | QQWTSYPLT |
| | NO. 494 | MY | NO. 495 | | NO. 496 | |
| F2.151.13 | SEQ ID | RSSTGAVTT | SEQ ID | GTNNQAP | SEQ ID | ALWFSNHWV |
| | NO. 512 | SNYAN | NO. 513 | | NO. 514 | |
| F2.70.2 | SEQ ID | TLSSQHSSY | SEQ ID | LKKDGSHSTGD | SEQ ID | GVGDTIKEQF |
| | NO. 530 | IIE | NO. 531 | | NO. 532 | VYV |
| F2.104.10 | SEQ ID | TLSSQHSSY | SEQ ID | VKKDGSHSTGD | SEQ ID | GVGDTIKEQF |
| | NO. 548 | IIE | NO. 549 | | NO. 550 | VYV |
| F2.180.16 | SEQ ID | TLSSQYSTY | SEQ ID | LKHDGSRHTGD | SEQ ID | GVGDTIKEQF |
| | NO. 566 | TIG | NO. 567 | | NO. 568 | MYV |
| F2.121.9 | SEQ ID | RSNTGAVT | SEQ ID | GTSNRAP | SEQ ID | ALWYSTHWV |
| | NO. 584 | TSNYAN | NO. 585 | | NO. 586 | |
| F2.173.9 | SEQ ID | TLSSQHSTY | SEQ ID | IKKDGSHYTGD | SEQ ID | GVSDMIKDQ |
| | NO. 602 | TIE | NO. 603 | | NO. 604 | FVYV |
| F2.343.16 | SEQ ID | RSSTGAVTT | SEQ ID | GTYNRVP | SEQ ID | ALWYSNHFW |
| | NO. 620 | SNYAN | NO. 621 | | NO. 622 | V |
| F2.205.9 | SEQ ID | RSSTGAVTT | SEQ ID | GTNNRAP | SEQ ID | ALWYSNHFI |
| | NO. 638 | RNYAN | NO. 639 | | NO. 640 | |
| F2.99.1 | SEQ ID | TLSRQHSA | SEQ ID | VKKDGSHSTGD | SEQ ID | GVGDTIKEHF |
| | NO. 656 | YTIE | NO. 657 | | NO. 658 | V |
| F2.127.11 | SEQ ID | TLSSQHSTY | SEQ ID | LKKDGSHSTGD | SEQ ID | GVSDTIKEQF |
| | NO. 674 | TIE | NO. 675 | | NO. 676 | VYV |
| F2.55.1 | SEQ ID | TLSSQHSAY | SEQ ID | LKKDGSHSTGD | SEQ ID | GVGDTVKEQ |
| | NO. 692 | TVE | NO. 693 | | NO. 694 | FV |
| F2.42.9 | SEQ ID | TLSSQHSAY | SEQ ID | LKKDGSHSTGD | SEQ ID | GVGDTIKEQ |
| | NO. 710 | TIE | NO. 711 | | NO. 712 | YV |

[symbol]

indicates data missing or illegible when filed

The heavy chain constant region containing a signal peptide and murine antibody IgG1 (SEQ ID NO: 10)

ASTKGPSVFPLAPSSKSTSGGTAALGCLVKDYFPEPVTVSWYSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQPREPQVYTLPP
SREEMTKNQVSLTCLVKGFYPSDTAVEWESNGQPENNYKTTPPVLDSDGSFFLYSKLTVDKSRWQQGNVFSCSVMHEALH
NHYTQKSLSLSPGK

The Kappa light chain constant region containing a signal peptide and human antibody IgG1 (SEQ ID NO: 11)

[symbol]

[symbol]

[symbol]

indicates data missing or illegible when filed

The Lambda light chain constant region containing a signal peptide and human antibody IgG1 (SEQ ID NO: 12)

[symbol]

[symbol]

[symbol]

indicates data missing or illegible when filed

### EXAMPLE 5

### Identification of CD22 Human-Murine Chimeric Antibodies

### 5.1 Binding of Chimeric Antibodies to CD22 Protein Detected By Enzyme-Linked Immunosorbent Assay (ELISA)

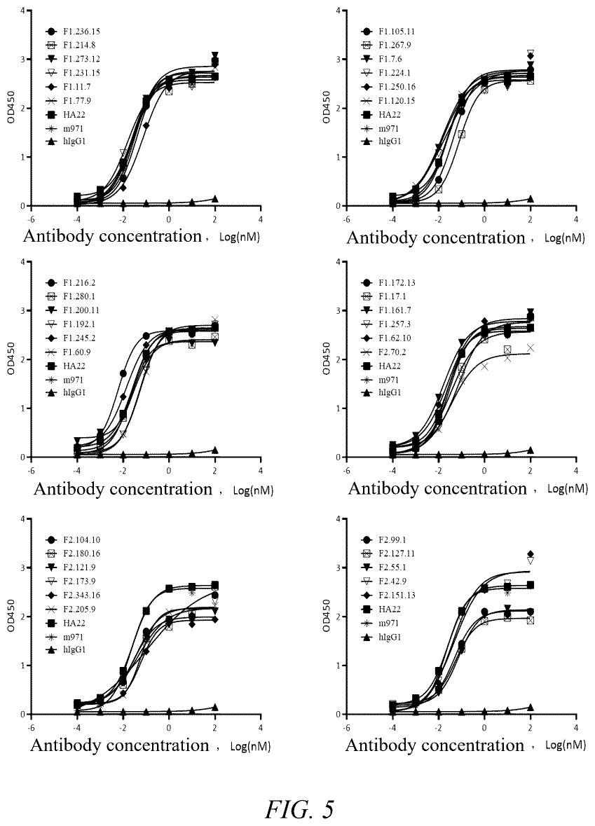

In order to detect the binding activity of CD22 human-murine chimeric antibodies to CD22 protein, the purified human CD22-ECD-His protein obtained in Example 2 was diluted with PBS to a final concentration of 2 μg/mL, and then added to 96-well ELISA plate at 100 μl/well. The plate was sealed with plastic film and incubated overnight at 4° C., the plate was washed 2 times with PBS the next day, and then a blocking solution [PBS+2% (w/w) BSA] was added for blocking at room temperature for 2 hours. The blocking solution was poured off, and 100 nM of serially diluted chimeric antibodies or negative control antibody was added at 50 μl/well. After incubation at 37° C. for 2 hours, the plate was washed 3 times with PBS. HRP (horseradish peroxidase)-labeled secondary antibody (purchased from Sigma, catalog number: A0170) was added, and incubated at 37° C. for 2 hours, and the plate was washed 5 times with PBS. TMB substrate was added at 50 μl/well, and incubated at room temperature for 30 minutes, then a stop solution (1.0 N HCl) was added at 50 μl/well. An ELISA plate reader (Multimode Plate Reader, EnSight, purchased from Perkin Elmer) was used to read the OD450 nm value, and the ELISA results of the chimeric antibodies and human CD22-ECD protein are shown in FIG. 5 and Table 11. Table 11 shows that the purified antibodies are all can bind to human CD22-ECD at ELISA level. The negative control antibody hIgG1 was the antibody anti-hel-hIgG1 (purchased from Biointron, catalog number: B117901) against chicken egg lysozyme. The data in the table are OD450 nm values.

TABLE 11
Binding reaction of chimeric antibodies
with human CD22 protein detected by ELISA

| | OD450 | | | | | | | |
|---|---|---|---|---|---|---|---|---|
| | Antibody concentration (nM) | | | | | | | |
| Antibody | 100.00 | 10.00 | 0.10 | 0.01 | 0.001 | 0.0001 | 0.00001 | Blank control |
| F1.236.15 | 2.98 | 2.62 | 2.51 | 2.05 | 0.57 | 0.14 | 0.09 | 0.08 |
| F1.214.8 | 2.95 | 2.50 | 2.34 | 2.14 | 0.81 | 0.18 | 0.08 | 0.06 |
| F1.273.12 | 3.07 | 2.72 | 2.58 | 2.20 | 0.77 | 0.17 | 0.07 | 0.06 |
| F1.231.15 | 2.64 | 2.44 | 2.42 | 2.14 | 1.08 | 0.27 | 0.13 | 0.06 |
| F1.11.7 | 2.89 | 2.63 | 2.42 | 1.64 | 0.37 | 0.11 | 0.06 | 0.06 |
| F1.77.9 | 2.84 | 2.64 | 2.58 | 2.12 | 0.69 | 0.19 | 0.11 | 0.06 |
| F1.105.11 | 2.79 | 2.68 | 2.52 | 1.93 | 0.53 | 0.14 | 0.08 | 0.07 |
| F1.267.9 | 2.56 | 2.55 | 2.39 | 1.47 | 0.34 | 0.10 | 0.06 | 0.07 |
| F1.7.6 | 2.88 | 2.46 | 2.51 | 2.20 | 1.19 | 0.34 | 0.15 | 0.08 |
| F1.224.1 | 3.12 | 2.43 | 2.37 | 2.21 | 1.08 | 0.34 | 0.17 | 0.07 |
| F1.250.16 | 3.07 | 2.56 | 2.58 | 2.24 | 0.86 | 0.21 | 0.12 | 0.06 |
| F1.120.15 | 2.89 | 2.61 | 2.63 | 2.28 | 1.21 | 0.42 | 0.12 | 0.06 |
| F1.216.2 | 2.69 | 2.53 | 2.55 | 2.48 | 1.71 | 0.45 | 0.20 | 0.06 |
| F1.280.1 | 2.46 | 2.30 | 2.38 | 1.92 | 0.81 | 0.24 | 0.13 | 0.06 |
| F1.200.11 | 2.34 | 2.33 | 2.40 | 2.00 | 0.85 | 0.51 | 0.33 | 0.07 |
| F1.192.1 | 2.71 | 2.57 | 2.52 | 1.79 | 0.46 | 0.10 | 0.06 | 0.07 |
| F1.245.2 | 2.70 | 2.54 | 2.57 | 2.30 | 1.24 | 0.44 | 0.22 | 0.07 |
| F1.60.9 | 2.82 | 2.55 | 2.59 | 1.75 | 0.47 | 0.13 | 0.09 | 0.08 |
| F1.172.13 | 2.87 | 2.51 | 2.53 | 2.11 | 0.74 | 0.17 | 0.10 | 0.10 |
| F1.17.1 | 2.86 | 2.21 | 2.41 | 1.85 | 0.75 | 0.20 | 0.11 | 0.10 |
| F1.161.7 | 2.97 | 2.63 | 2.61 | 2.34 | 1.22 | 0.43 | 0.25 | 0.06 |
| F1.257.3 | 2.83 | 2.64 | 2.49 | 1.66 | 0.60 | 0.28 | 0.20 | 0.07 |
| F1.62.10 | 2.93 | 2.67 | 2.79 | 2.16 | 1.07 | 0.37 | 0.24 | 0.06 |
| F2.70.2 | 2.24 | 2.03 | 1.86 | 1.56 | 0.57 | 0.18 | 0.09 | 0.07 |
| F2.104.10 | 2.44 | 2.00 | 1.94 | 1.70 | 0.66 | 0.32 | 0.22 | 0.06 |
| F2.180.16 | 2.61 | 2.08 | 1.80 | 1.55 | 0.61 | 0.31 | 0.21 | 0.06 |
| F2.121.9 | 2.11 | 1.92 | 1.84 | 1.60 | 0.69 | 0.33 | 0.23 | 0.06 |
| F2.173.9 | 2.32 | 1.97 | 1.93 | 1.52 | 0.65 | 0.32 | 0.18 | 0.06 |
| F2.343.16 | 1.94 | 1.84 | 1.93 | 1.29 | 0.43 | 0.25 | 0.18 | 0.06 |
| F2.205.9 | 2.22 | 2.11 | 2.10 | 1.48 | 0.40 | 0.24 | 0.19 | 0.06 |
| F2.99.1 | 2.10 | 2.06 | 2.10 | 1.45 | 0.51 | 0.27 | 0.08 | 0.06 |
| F2.127.11 | 1.92 | 1.98 | 1.91 | 1.34 | 0.50 | 0.26 | 0.21 | 0.06 |
| F2.55.1 | 2.08 | 2.16 | 2.03 | 1.30 | 0.44 | 0.23 | 0.16 | 0.07 |
| F2.42.9 | 3.14 | 2.68 | 2.58 | 1.95 | 0.68 | 0.19 | 0.09 | 0.08 |
| F2.151.13 | 3.28 | 2.60 | 2.55 | 2.10 | 0.64 | 0.20 | 0.10 | 0.07 |
| HA22 | 2.65 | 2.61 | 2.57 | 2.12 | 0.89 | 0.33 | 0.19 | 0.07 |
| m971 | 2.63 | 2.48 | 2.58 | 2.12 | 0.83 | 0.17 | 0.08 | 0.06 |
| hIgG1 | 0.15 | 0.08 | 0.06 | 0.05 | 0.06 | 0.06 | 0.05 | 0.06 |

### 5.2 The Binding of Chimeric Antibodies to Different CD22 Expressing Cells Detected by Flow Cytometry (FACS)

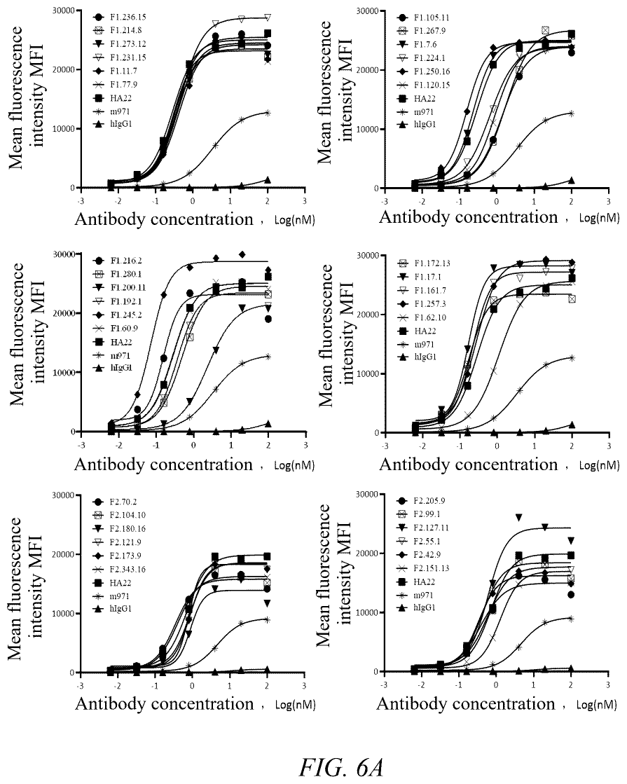

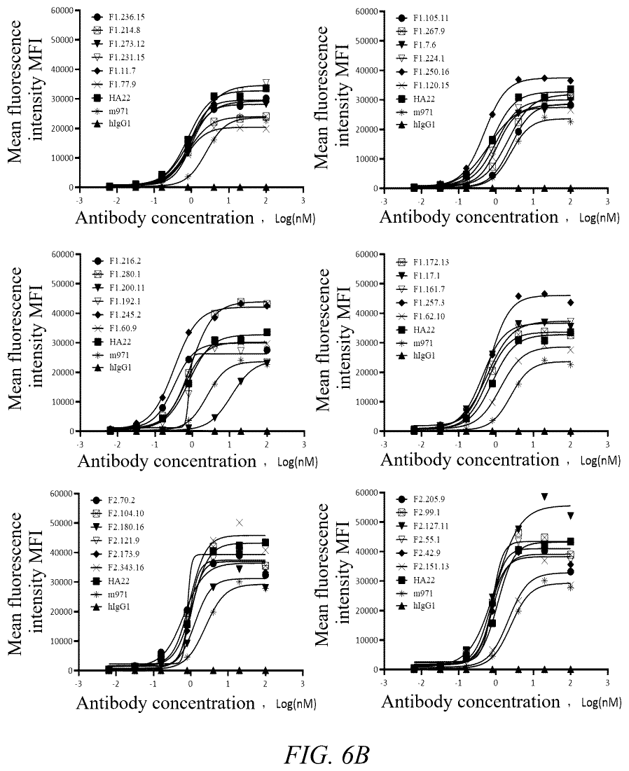

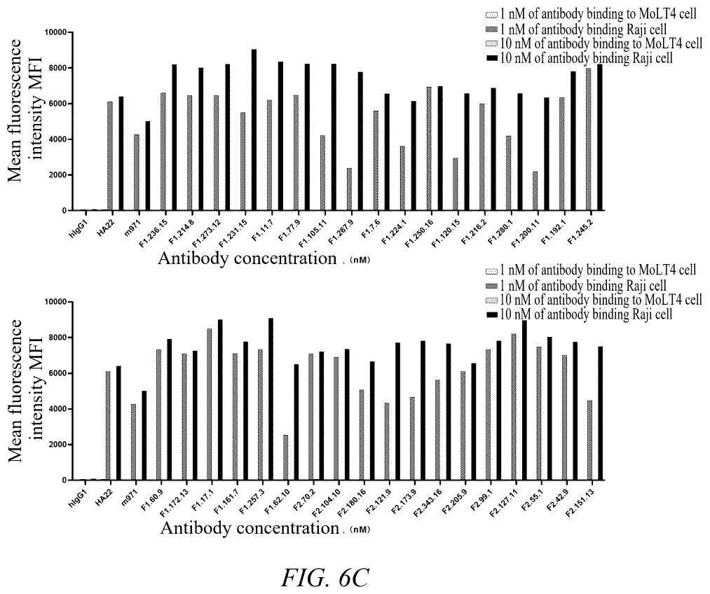

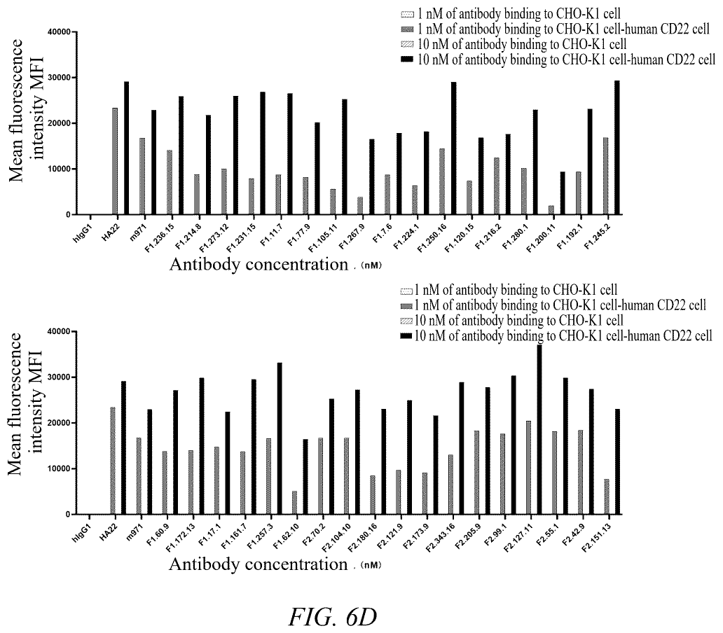

The required cells were scale-up cultured in a T-75 cell culture flask to the logarithmic growth phase. For the adherent cell CHO-K1, the medium was aspirated, the cells were washed 2 times with PBS buffer, and then digested with trypsin. After the digestion was terminated, the cells were washed 2 times with PBS buffer. For suspension cell Raji, the medium supernatant was directly centrifuged and discarded, and the cell pellet was washed 2 times with PBS. After counting the cells in the previous step, the cell pellet was resuspended with [PBS+2% (w/w) BSA] blocking solution to 2×10⁶ cells/ml, and added to a 96-well FACS reaction plate at 50 μl/well, and then the chimeric antibody test sample was added at 50 μl/well, and incubated on ice for 2 hours. The mixture was centrifuged and washed 3 times with PBS buffer, Alexa Flour 488-labeled secondary antibody (purchased from Invitrogen, catalog number: A-11013) was added at 50 μl/well, and incubated on ice for 1 hour. The obtained mixture was centrifuged and washed 5 times with PBS, and FACS (FACS Canto™, purchased from BD Company) was used for detection and result analysis. Data analysis was performed by software (CellQuest) to obtain the mean fluorescence density (MFI)of the cells. And then software (GraphPad Prism8) was used for analysis, data fitting, and EC50 value calculation. The analysis results are shown in Tables 12-13 and FIGS. 6A-6B. The chimeric antibodies all can bind to human CD22 protein on the surface of Raji cells and CHO-K1-human CD22 2C4 cells (FIGS. 6A-6B). The same method was used to detect the binding of the chimeric antibodies to endogenous CD22-negative cell MoLT4 cells (purchased from ATCC, CRL-1582) and CHO-K1 cells. The results are shown in FIGS. 6C-6D: FIGS. 6C-6D do not show the histograms of binding to MoLT4 cells and CHO-K1 cells. It can be seen that all the chimeric antibodies do not bind to MoLT4 cells and CHO-K1 cells, and have good specificity.

TABLE 12
Binding reaction of chimeric antibodies with Raji
and CHO-K1-human CD22 2C4 cells detected by FACS

| | Raji | | CHO-K1-human CD22 2C4 | |
|---|---|---|---|---|
| | Maximum | | Maximum | |
| | mean | | mean | |
| Designation | fluorescence | Ec50 | fluorescence | Ec50 |
| of antibody | intensity | (nM) | intensity | (nM) |
| F1.236.15 | 26025 | 0.34 | 30207 | 0.81 |
| F1.214.8 | 24067 | 0.33 | 24120 | 0.81 |
| F1.273.12 | 25310 | 0.38 | 28225 | 0.99 |
| F1.231.15 | 28757 | 0.45 | 35404 | 1.18 |
| F1.11.7 | 25492 | 0.42 | 29596 | 0.89 |
| F1.77.9 | 24148 | 0.29 | 20158 | 0.71 |
| F1.105.11 | 24407 | 1.39 | 28288 | 2.53 |
| F1.267.9 | 26741 | 1.66 | 31189 | 2.08 |
| F1.7.6 | 25861 | 0.23 | 29720 | 0.63 |
| F1.224.1 | 23595 | 0.65 | 30372 | 0.99 |
| F1.250.16 | 24999 | 0.16 | 37373 | 0.49 |
| F1.120.15 | 23746 | 0.89 | 27851 | 1.07 |
| F1.216.2 | 25288 | 0.16 | 31494 | 0.32 |
| F1.280.1 | 25039 | 0.5 | 43819 | 0.92 |
| F1.200.11 | 20844 | 2.41 | 23041 | 11.2 |
| F1.192.1 | 24398 | 0.37 | 27170 | 0.81 |
| F1.245.2 | 29927 | 0.07 | 43303 | 0.33 |
| F1.60.9 | 25323 | 0.29 | 30139 | 0.61 |
| F1.172.13 | 23765 | 0.17 | 33783 | 0.59 |
| F1.17.1 | 28521 | 0.17 | 36915 | 0.44 |
| F1.161.7 | 27853 | 0.22 | 37135 | 0.59 |
| F1.257.3 | 29336 | 0.27 | 46611 | 0.68 |
| F1.62.10 | 25601 | 1.1 | 29180 | 1.31 |
| HA22 | 25015 | 0.29 | 32749 | 0.81 |
| m971 | 13043 | 3.32 | 23659 | 2.45 |
| hIgG1 | 2772 | Negative | 5221 | Negative |

TABLE 13
Binding reaction of chimeric antibodies with Raji
and CHO-K1-human CD22 2C4 cells detected by FACS

| | Raji | | CHO-K1-human CD22 2C4 | |
|---|---|---|---|---|
| | Maximum | | Maximum | |
| | mean | | mean | |
| Designation | fluorescence | Ec50 | fluorescence | Ec50 |
| of antibody | intensity | (nM) | intensity | (nM) |
| F2.70.2 | 16564 | 0.35 | 39021 | 0.76 |
| F2.104.10 | 16258 | 0.4 | 40596 | 0.88 |
| F2.180.16 | 15686 | 0.88 | 34374 | 1.63 |
| F2.121.9 | 18917 | 0.86 | 39749 | 1.09 |
| F2.173.9 | 18947 | 0.84 | 40264 | 1.23 |
| F2.343.16 | 19332 | 0.8 | 50169 | 1.15 |
| F2.205.9 | 15548 | 0.38 | 40245 | 0.72 |
| F2.99.1 | 18104 | 0.59 | 44796 | 0.83 |
| F2.127.11 | 24364 | 0.59 | 58514 | 1.22 |
| F2.55.1 | 18574 | 0.41 | 42004 | 0.82 |
| F2.42.9 | 16701 | 0.33 | 40953 | 0.87 |
| F2.151.13 | 18763 | 1.26 | 36975 | 3.28 |
| HA22 | 19668 | 0.71 | 43414 | 1.1 |
| m971 | 8917 | 4.61 | 30114 | 2.86 |
| hIgG1 | 564 | Negative | 150 | Negative |

### EXAMPLE 6

### Detection of Species Cross-Binding Activity of Chimeric Antibodies

### 6.1 Binding of Chimeric Antibodies to CD22 Proteins of Different Species Detected by ELISA

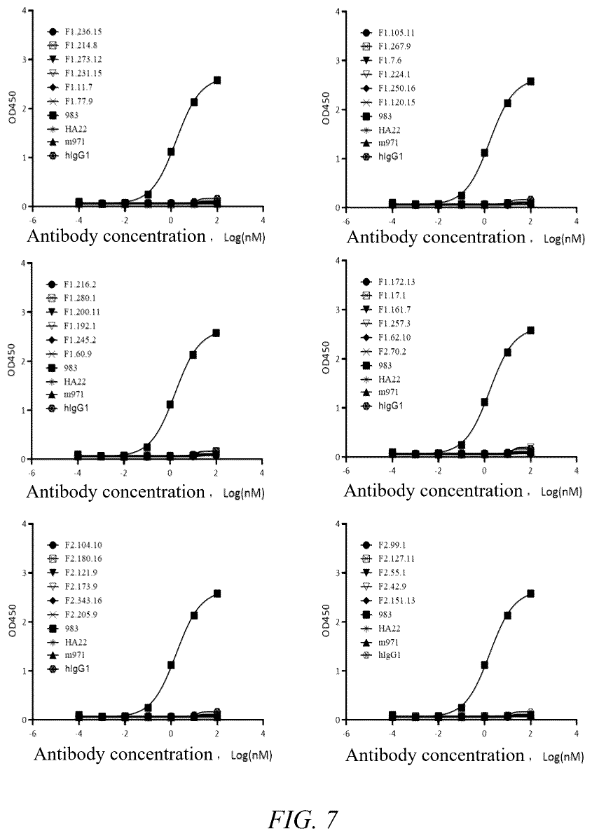

In order to detect the species cross-binding activity of the chimeric antibodies, an ELISA plate was coated with commercial murine CD22 protein (ACROBiosystems, catalog number: SI2-M52Ha) and monkey CD22 protein (ACROBiosystems, catalog number: SI2-R52Ha), respectively, and the ELISA detection was performed according to the method in Example 5.1. The ELISA results of the chimeric antibodies and the murine CD22-ECD are shown in FIG. 7 and Table 14. Table 14 shows that all the purified chimeric antibodies do not bind to murine CD22-ECD at the ELISA level. The negative control is hIgG1, and 983 is the serum of human CD22-ECD-His immunized mice as a positive control, and the data in the table are OD450 nm values.

TABLE 14
Binding reaction of chimeric antibodies with
murine CD22 protein detected by ELISA

| | OD450 | | | | | | | |
|---|---|---|---|---|---|---|---|---|
| Designation of | Antibody concentration (nM) | | | | | | | |
| antibody | 100 | 10 | 0.1 | 0.01 | 0.001 | 0.0001 | 0.00001 | Blank control |
| F1.236.15 | 0.07 | 0.08 | 0.07 | 0.07 | 0.07 | 0.07 | 0.08 | 0.08 |
| F1.214.8 | 0.06 | 0.05 | 0.05 | 0.05 | 0.05 | 0.05 | 0.06 | 0.07 |
| F1.273.12 | 0.06 | 0.05 | 0.05 | 0.05 | 0.05 | 0.05 | 0.05 | 0.07 |
| F1.231.15 | 0.06 | 0.06 | 0.05 | 0.05 | 0.05 | 0.05 | 0.06 | 0.06 |
| F1.11.7 | 0.07 | 0.05 | 0.05 | 0.06 | 0.05 | 0.05 | 0.06 | 0.07 |
| F1.77.9 | 0.08 | 0.06 | 0.05 | 0.05 | 0.05 | 0.06 | 0.06 | 0.06 |
| F1.105.11 | 0.08 | 0.05 | 0.05 | 0.05 | 0.05 | 0.05 | 0.05 | 0.06 |
| F1.267.9 | 0.07 | 0.08 | 0.06 | 0.06 | 0.07 | 0.07 | 0.07 | 0.07 |
| F1.7.6 | 0.07 | 0.06 | 0.06 | 0.06 | 0.06 | 0.06 | 0.06 | 0.07 |
| F1.224.1 | 0.07 | 0.10 | 0.05 | 0.06 | 0.05 | 0.05 | 0.06 | 0.06 |
| F1.250.16 | 0.06 | 0.06 | 0.05 | 0.05 | 0.05 | 0.05 | 0.06 | 0.06 |
| F1.120.15 | 0.06 | 0.06 | 0.05 | 0.05 | 0.05 | 0.05 | 0.06 | 0.07 |
| F1.216.2 | 0.08 | 0.06 | 0.05 | 0.05 | 0.05 | 0.06 | 0.06 | 0.07 |
| F1.280.1 | 0.16 | 0.07 | 0.05 | 0.05 | 0.05 | 0.05 | 0.06 | 0.07 |
| F1.200.11 | 0.07 | 0.06 | 0.05 | 0.05 | 0.05 | 0.05 | 0.06 | 0.07 |
| F1.192.1 | 0.08 | 0.08 | 0.07 | 0.07 | 0.06 | 0.07 | 0.07 | 0.08 |
| F1.245.2 | 0.09 | 0.07 | 0.06 | 0.06 | 0.06 | 0.07 | 0.06 | 0.07 |
| F1.60.9 | 0.08 | 0.06 | 0.05 | 0.06 | 0.05 | 0.06 | 0.06 | 0.07 |
| F1.172.13 | 0.08 | 0.06 | 0.05 | 0.05 | 0.05 | 0.06 | 0.06 | 0.07 |
| F1.17.1 | 0.07 | 0.06 | 0.05 | 0.05 | 0.05 | 0.05 | 0.06 | 0.07 |
| F1.161.7 | 0.08 | 0.06 | 0.05 | 0.05 | 0.05 | 0.05 | 0.06 | 0.07 |
| F1.257.3 | 0.20 | 0.07 | 0.05 | 0.05 | 0.05 | 0.05 | 0.06 | 0.07 |
| F1.62.10 | 0.08 | 0.06 | 0.05 | 0.05 | 0.05 | 0.06 | 0.07 | 0.07 |
| F2.70.2 | 0.10 | 0.07 | 0.06 | 0.06 | 0.06 | 0.06 | 0.06 | 0.07 |
| F2.104.10 | 0.07 | 0.07 | 0.06 | 0.07 | 0.06 | 0.06 | 0.05 | 0.07 |
| F2.180.16 | 0.05 | 0.06 | 0.05 | 0.05 | 0.05 | 0.07 | 0.06 | 0.06 |
| F2.121.9 | 0.06 | 0.06 | 0.05 | 0.05 | 0.06 | 0.06 | 0.06 | 0.07 |
| F2.173.9 | 0.09 | 0.06 | 0.05 | 0.05 | 0.05 | 0.05 | 0.06 | 0.09 |
| F2.343.16 | 0.07 | 0.06 | 0.05 | 0.05 | 0.05 | 0.05 | 0.06 | 0.07 |
| F2.205.9 | 0.07 | 0.06 | 0.05 | 0.05 | 0.05 | 0.05 | 0.06 | 0.06 |
| F2.99.1 | 0.07 | 0.06 | 0.05 | 0.06 | 0.05 | 0.05 | 0.06 | 0.07 |
| F2.127.11 | 0.10 | 0.07 | 0.06 | 0.05 | 0.05 | 0.06 | 0.06 | 0.06 |
| F2.55.1 | 0.09 | 0.06 | 0.06 | 0.05 | 0.05 | 0.06 | 0.06 | 0.06 |
| F2.42.9 | 0.09 | 0.08 | 0.08 | 0.07 | 0.07 | 0.08 | 0.08 | 0.09 |
| F2.151.13 | 0.09 | 0.08 | 0.07 | 0.07 | 0.07 | 0.07 | 0.07 | 0.07 |
| 983* | 2.58 | 2.13 | 1.12 | 0.25 | 0.08 | 0.06 | 0.10 | 0.09 |
| HA22 | 0.10 | 0.07 | 0.06 | 0.06 | 0.05 | 0.06 | 0.06 | 0.07 |
| m971 | 0.11 | 0.08 | 0.07 | 0.07 | 0.06 | 0.07 | 0.07 | 0.08 |
| hIgG1 | 0.16 | 0.09 | 0.08 | 0.08 | 0.08 | 0.07 | 0.08 | 0.08 |

*983 serum was 5-fold serially diluted from 1:100.

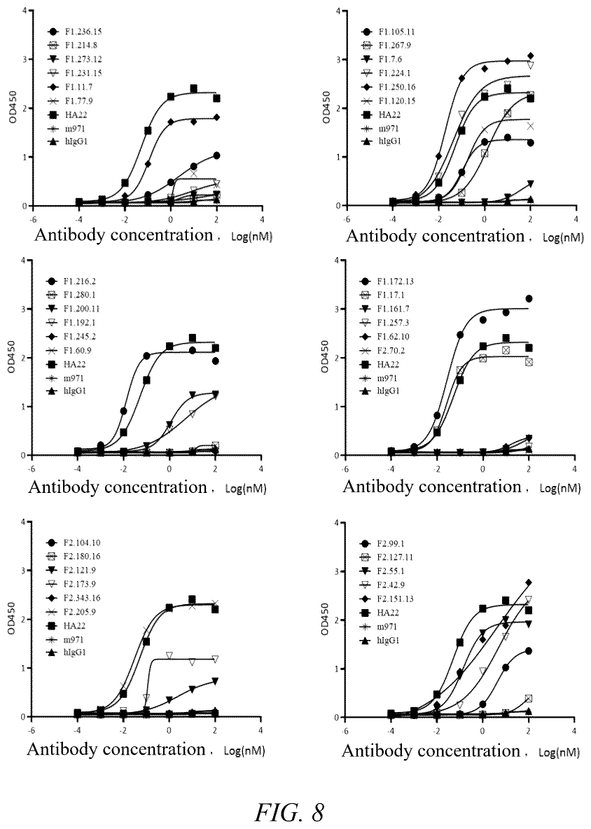

The ELISA results of the chimeric antibodies and the monkey CD22-ECD are shown in FIG. 8 and Table 15. Table 15 shows that F1.236.15, F1.11.7, F1.105.11, F1.267.9, F1.224.1, F1.250.16, F1.120.15, F1.216.2, F1.200.11, F1.192.1, F1.172.13, F1.17.1, F2.121.9, F2.173.9, F1.205.9, F2.99.1, F2.55.1, F2.42.9 and F2.151.13 have binding activity to the monkey CD22-ECD protein.

TABLE 15
Binding reaction of chimeric antibodies with
monkey CD22 protein detected by ELISA

| | OD450 | | | | | | | |
|---|---|---|---|---|---|---|---|---|
| Designation of | Antibody concentration (nM) | | | | | | | |
| antibody | 100 | 10 | 0.1 | 0.01 | 0.001 | 0.0001 | 0.00001 | Blank control |
| F1.236.15 | 1.03 | 0.81 | 0.49 | 0.23 | 0.08 | 0.06 | 0.06 | 0.07 |
| F1.214.8 | 0.23 | 0.15 | 0.12 | 0.08 | 0.06 | 0.07 | 0.06 | 0.06 |
| F1.273.12 | 0.23 | 0.22 | 0.09 | 0.07 | 0.06 | 0.06 | 0.06 | 0.06 |
| F1.231.15 | 0.45 | 0.31 | 0.17 | 0.09 | 0.06 | 0.06 | 0.06 | 0.06 |
| F1.11.7 | 1.82 | 1.72 | 1.72 | 0.86 | 0.21 | 0.08 | 0.06 | 0.06 |
| F1.77.9 | 0.44 | 0.67 | 0.14 | 0.08 | 0.06 | 0.06 | 0.06 | 0.06 |
| F1.105.11 | 1.29 | 1.40 | 1.31 | 0.68 | 0.17 | 0.07 | 0.06 | 0.06 |
| F1.267.9 | 2.27 | 1.89 | 1.07 | 0.27 | 0.10 | 0.07 | 0.06 | 0.07 |
| F1.7.6 | 0.44 | 0.17 | 0.07 | 0.06 | 0.06 | 0.06 | 0.06 | 0.06 |
| F1.224.1 | 2.87 | 2.48 | 2.30 | 1.86 | 0.60 | 0.15 | 0.08 | 0.06 |
| F1.250.16 | 3.08 | 2.97 | 2.81 | 2.62 | 1.02 | 0.23 | 0.10 | 0.07 |
| F1.120.15 | 1.64 | 1.90 | 1.56 | 0.65 | 0.16 | 0.07 | 0.06 | 0.06 |
| F1.216.2 | 1.94 | 2.15 | 2.24 | 2.04 | 0.91 | 0.21 | 0.09 | 0.06 |
| F1.280.1 | 0.20 | 0.08 | 0.06 | 0.05 | 0.06 | 0.06 | 0.06 | 0.07 |
| F1.200.11 | 1.25 | 1.23 | 0.62 | 0.19 | 0.08 | 0.06 | 0.06 | 0.06 |
| F1.192.1 | 1.21 | 0.84 | 0.53 | 0.19 | 0.07 | 0.07 | 0.07 | 0.07 |
| F1.245.2 | 0.08 | 0.06 | 0.06 | 0.06 | 0.06 | 0.06 | 0.06 | 0.07 |
| F1.60.9 | 0.09 | 0.07 | 0.06 | 0.05 | 0.05 | 0.06 | 0.06 | 0.06 |
| F1.172.13 | 3.21 | 2.93 | 2.78 | 2.47 | 0.82 | 0.18 | 0.09 | 0.07 |
| F1.17.1 | 1.91 | 2.16 | 1.99 | 1.75 | 0.51 | 0.13 | 0.07 | 0.06 |
| F1.161.7 | 0.34 | 0.13 | 0.06 | 0.05 | 0.05 | 0.05 | 0.06 | 0.06 |
| F1.257.3 | 0.18 | 0.07 | 0.06 | 0.05 | 0.05 | 0.06 | 0.06 | 0.07 |
| F1.62.10 | 0.36 | 0.18 | 0.07 | 0.05 | 0.05 | 0.06 | 0.06 | 0.06 |
| F2.70.2 | 0.11 | 0.08 | 0.06 | 0.06 | 0.07 | 0.06 | 0.06 | 0.06 |
| F2.104.10 | 0.07 | 0.07 | 0.05 | 0.06 | 0.05 | 0.05 | 0.05 | 0.06 |
| F2.180.16 | 0.06 | 0.06 | 0.05 | 0.05 | 0.05 | 0.05 | 0.05 | 0.06 |
| F2.121.9 | 0.72 | 0.56 | 0.34 | 0.13 | 0.06 | 0.05 | 0.05 | 0.06 |
| F2.173.9 | 1.18 | 1.12 | 1.25 | 0.38 | 0.12 | 0.06 | 0.06 | 0.06 |
| F2.343.16 | 0.08 | 0.06 | 0.05 | 0.05 | 0.05 | 0.05 | 0.05 | 0.06 |
| F2.205.9 | 2.33 | 2.28 | 2.21 | 1.78 | 0.63 | 0.16 | 0.08 | 0.06 |
| F2.99.1 | 1.37 | 1.03 | 0.27 | 0.07 | 0.05 | 0.05 | 0.05 | 0.06 |
| F2.127.11 | 0.39 | 0.09 | 0.05 | 0.05 | 0.05 | 0.05 | 0.05 | 0.06 |
| F2.55.1 | 1.91 | 2.00 | 1.73 | 0.85 | 0.18 | 0.07 | 0.06 | 0.08 |
| F2.42.9 | 2.42 | 1.65 | 0.95 | 0.25 | 0.09 | 0.07 | 0.07 | 0.08 |
| F2.151.13 | 2.77 | 1.89 | 1.60 | 0.94 | 0.27 | 0.10 | 0.07 | 0.07 |
| HA22 | 2.20 | 2.41 | 2.24 | 1.54 | 0.47 | 0.14 | 0.08 | 0.06 |
| m971 | 0.12 | 0.09 | 0.07 | 0.07 | 0.06 | 0.07 | 0.07 | 0.08 |
| hIgG1 | 0.13 | 0.10 | 0.07 | 0.07 | 0.07 | 0.07 | 0.07 | 0.07 |

### 6.2 The Binding of Chimeric Antibodies to Monkey CD22 Expressing Cells Detected by FACS

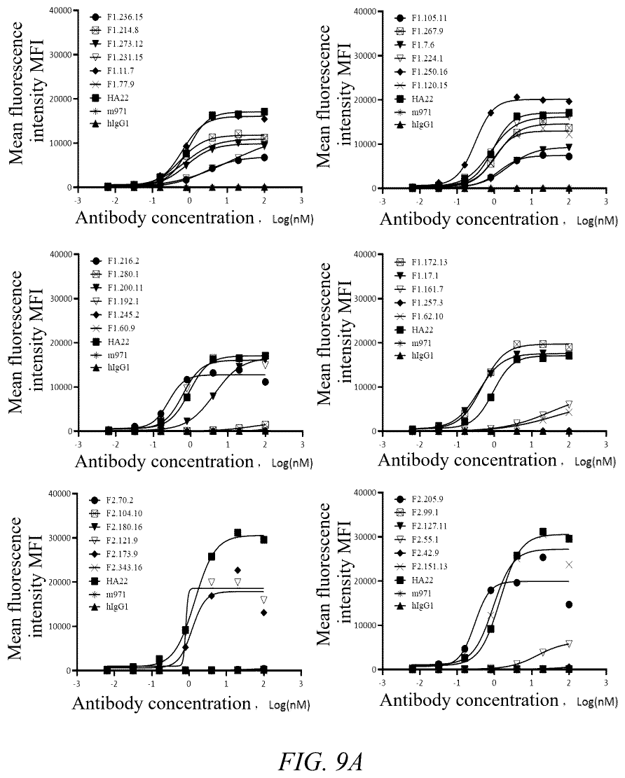

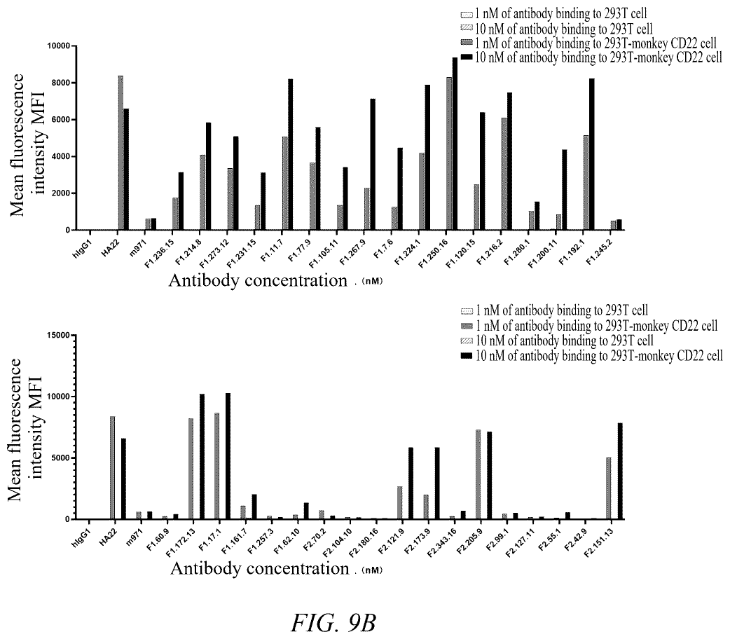

HEK293T-monkey CD22 cells were subjected to FACS detection and data analysis according to the method in Example 5.2. The analysis results are shown in Tables 16-17 and FIG. 9A, except for F1.280.1, F1.245.2, F1.60.9, F1.257.3, F2.70.2, F2.104.10, F2.180.16, F2.343.16, F2.99.1, F2.127.11 and F2.42.9, other chimeric antibodies have binding activity to HEK293T cells overexpressing monkey CD22, and the EC50 shows that the highest binding activity is up to 0.26 nM. The same method was used to detect the binding of 1 nm and 10 nm of the chimeric antibodies to HEK293T cells at the same time, and the results are shown in FIG. 9B: FIG. 9B does not show the histograms of binding to HEK293T cells. It can be seen that all the chimeric antibodies do not bind to HEK293T cells, and have good specificity.

TABLE 16
Binding reaction of chimeric antibodies with
HEK293T-monkey CD22 cells detected by FACS

HEK293T-monkey CD22

| | Maximum mean | |
|---|---|---|
| Designation of | fluorescence | Ec50 |
| antibody | intensity | (nM) |
| F1.236.15 | 6754 | 2.47 |
| F1.214.8 | 12120 | 0.55 |
| F1.273.12 | 9998 | 0.82 |
| F1.231.15 | 9254 | 12.34 |
| F1.11.7 | 16077 | 0.63 |
| F1.77.9 | 10879 | 0.71 |
| F1.105.11 | 7528 | 1.3 |
| F1.267.9 | 15314 | 1.15 |
| F1.7.6 | 9223 | 2.22 |
| F1.224.1 | 16267 | 0.79 |
| F1.250.16 | 19655 | 0.31 |
| F1.120.15 | 13565 | 0.95 |
| F1.216.2 | 13880 | 0.26 |
| F1.280.1 | 1488 | Negative |
| F1.200.11 | 16187 | 4.32 |
| F1.192.1 | 16286 | 0.62 |
| F1.245.2 | 124 | Negative |
| F1.60.9 | 531 | Negative |
| F1.172.13 | 19039 | 0.47 |
| F1.17.1 | 17192 | 0.38 |
| F1.161.7 | 6027 | 31.2 |
| F1.257.3 | 147 | Negative |
| F1.62.10 | 4326 | 41.08 |
| HA22 | 17054 | 0.91 |
| m971 | 49 | Negative |
| hIgG1 | 129 | Negative |

TABLE 17
Binding reaction of chimeric antibodies with
HEK293T-monkey CD22 cells detected by FACS

HEK293T-monkey

| | Maximum mean | |
|---|---|---|
| Designation of | fluorescence | Ec50 |
| antibody | intensity | (nM) |
| F2.70.2 | 409 | Negative |
| F2.104.10 | 251 | Negative |
| F2.180.16 | 69 | Negative |
| F2.121.9 | 19947 | 0.97 |
| F2.173.9 | 22679 | 2.1 |
| F2.343.16 | 83 | Negative |
| F2.205.9 | 25370 | 0.4 |
| F2.99.1 | 144 | Negative |
| F2.127.11 | 109 | Negative |
| F2.55.1 | 5765 | 34.7 |
| F2.42.9 | 580 | Negative |
| F2.151.13 | 30502 | 1.26 |
| HA22 | 31169 | 1.59 |
| m971 | 104 | Negative |
| hIgG1 | 167 | Negative |

6.3 Binding of Chimeric Antibodies to Peripheral Blood B Cells of Cynomolgus Monkey (Latin Name: Macaca fascicularis) Detected by FACS

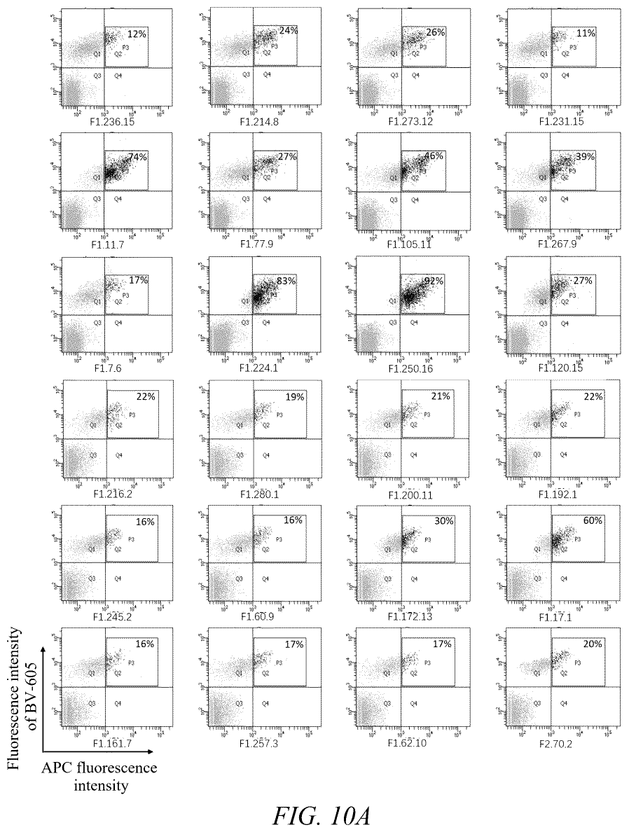

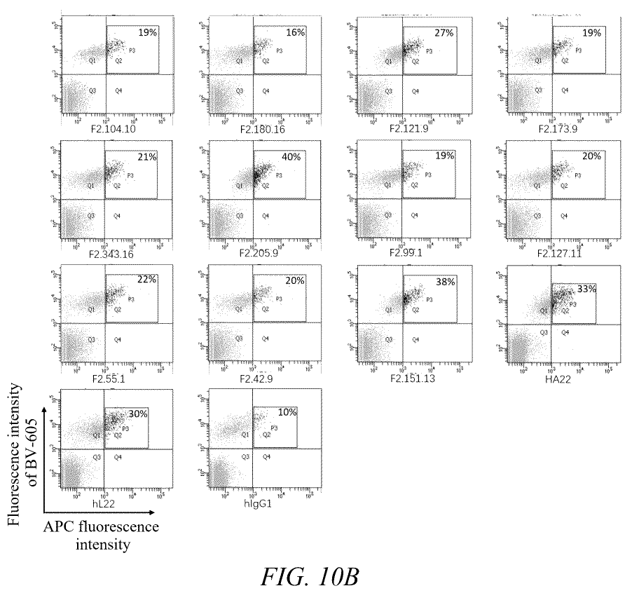

The monkey peripheral blood mononuclear cells were extracted from fresh cynomolgus monkey peripheral blood (purchased from Shanghai Medicilon Inc.) according to the instructions of Ficoll-Paque Plus (purchased from GE Healthcare, catalog number: 171440-02). After the cell suspension was centrifuged, the cells were resuspended in PBS containing 1% BSA, and then the cells were counted. At the same time, the murine antibody Brilliant Violet 605 anti-human CD20 (catalog number: 302334, purchased from Biolegend) with monkey CD20 cross-binding activity and the chimeric antibodies to be tested (1 nM, 10 nM and 100 nM) were added and incubated at room temperature for 1 hour. After washing the cells three times, APC-labeled secondary antibody anti-human IgG Fc (catalog number: 409306, purchased from Biolegend) was added. After incubation at room temperature in the dark for 30 minutes, the cells were washed 5 times, gently resuspended with PBS containing 1% BSA, and detected and analyzed by FACS (FACS Canto™, purchased from BD Company). Wherein CD20 was used as a marker of B cells, and the CD20-positive B cell population was gated, the proportion of chimeric antibody positive cells was analyzed, and the proportion of chimeric antibody positive cell population to B cell population was calculated after treatments with the chimeric antibodies at the concentrations of 100 nM, 10 nM and 1 nM, respectively. The results are shown in Table 18. The scatter plot of double-stained cells by Brilliant Violet 605-labeled CD20 and APC secondary antibody indirectly labeled chimeric antibody is shown in FIG. 10A-FIG. 10B (under the condition of 1 nM of chimeric antibody concentration). It can be seen from the results that F1.11.7, F1.77.9, F1.105.11, F1.267.9, F1.224.1, F1.250.16, F1.120.15, F1.17.1, F2.121.9 and F2.151.13 still bind to B cells of cynomolgus monkeys in a high proportion even under the condition of low concentration of 1 nM, and has equivalent or better binding activity than positive antibodies HA22 and hL22.

TABLE 18
Binding reaction of chimeric antibodies with
cynomolgus monkey B cells detected by FACS
Proportion of chimeric antibody positive cells to B cells (%)

| Designation of | Antibody concentration | | |
|---|---|---|---|
| antibody | 100 nM | 10 nM | 1 nM |
| F1.236.15 | 22 | 17 | 12 |
| F1.214.8 | 52 | 35 | 24 |
| F1.273.12 | 45 | 33 | 26 |
| F1.231.15 | 29 | 16 | 11 |
| F1.11.7 | 91 | 84 | 74 |
| F1.77.9 | 61 | 39 | 27 |
| F1.105.11 | 69 | 59 | 46 |
| F1.267.9 | 95 | 91 | 39 |
| F1.7.6 | 43 | 28 | 17 |
| F1.224.1 | 97 | 96 | 83 |
| F1.250.16 | 97 | 95 | 92 |
| F1.120.15 | 93 | 86 | 27 |
| F1.216.2 | 34 | 25 | 22 |
| F1.280.1 | 35 | 18 | 19 |
| F1.200.11 | 56 | 37 | 21 |
| F1.192.1 | 38 | 30 | 22 |
| F1.245.2 | 17 | 17 | 16 |
| F1.60.9 | 18 | 16 | 16 |
| F1.172.13 | 34 | 31 | 30 |
| F1.17.1 | 68 | 62 | 60 |
| F1.161.7 | 29 | 21 | 16 |
| F1.257.3 | 34 | 20 | 17 |
| F1.62.10 | 24 | 19 | 17 |
| F2.70.2 | 34 | 20 | 20 |
| F2.104.10 | 26 | 21 | 19 |
| F2.180.16 | 23 | 20 | 16 |
| F2.121.9 | 64 | 56 | 27 |
| F2.173.9 | 71 | 43 | 19 |
| F2.343.16 | 22 | 21 | 21 |
| F2.205.9 | 46 | 41 | 40 |
| F2.99.1 | 22 | 16 | 19 |
| F2.127.11 | 22 | 21 | 20 |
| F2.55.1 | 66 | 28 | 22 |
| F2.42.9 | 23 | 20 | 20 |
| F2.151.13 | 60 | 48 | 38 |
| HA22 | 93 | 89 | 33 |
| hL22 | 66 | 42 | 30 |
| hIgG1 | 10 | 8 | 10 |

### EXAMPLE 7

### CD22 Antibody Affinity Assay

### 7.1 Affinity Assay of Chimeric Antibodies to Human CD22-ECD-His Protein

Anti-human CD22 chimeric antibodies were captured using Protein A chips (GE Healthcare; 29-127-558). Sample buffer and running buffer were HBS-EP+(10 mM HEPES, 150 mM NaCl, 3 mM EDTA, 0.05% surfactant P20) (GE Healthcare; BR-1006-69). The flow-through cell was set to 25° C. The sample block was set to 16° C. Both were pretreated with the running buffer. In each cycle, the antibody to be tested was first captured with a Protein A chip, then a single concentration of CD22 antigen protein was injected. The binding and dissociation process of the antibody and the antigen protein was recorded, and finally Glycine pH 1.5 (GE Healthcare; BR-1003-54) was used to complete chip regeneration. Binding was measured by injecting different concentrations of recombinant human CD22-ECD His protein in solution for 240 s with a flow rate of 30 μL/min. The concentration started from 200 nM (see the detailed results for the actual concentration in the test) and was diluted at 1:1, making a total of 5 concentrations. The dissociation phase was monitored for up to 600 s and was triggered by switching from sample solution to running buffer. The surface was regenerated by washing with a 10 mM of glycine solution (pH 1.5) for 30 s at a flow rate of 30 μL/min. Bulk refractive index difference was corrected by subtracting the response obtained from the goat anti-human Fc surface. Blank injection was also subtracted (=double reference). For calculation of apparent KD and other kinetic parameters, Langmuir 1:1 model was used. The binding rate (ka), dissociation rate (kd) and binding affinity (KD) of the chimeric antibodies to human CD22-His protein are shown in Table 19, and the antibodies HA22 and m971 were used as positive l controls. As shown in Table 19, the highest affinity of the chimeric antibodies to human CD22 can reach 2.54E-10M.

TABLE 19
Affinity of chimeric antibodies to human
CD22 detected by SPR (biacore)

| Designation of | ka | kd | KD |
|---|---|---|---|
| antibody | (1/Ms) | (1/s) | (M) |
| F1.11.7 | 2.27E+05 | 7.80E−05 | 3.43E−10 |
| F1.105.11 | 2.99E+05 | 1.54E−04 | 5.14E−10 |
| F1.236.15 | 2.62E+05 | 1.56E−04 | 5.96E−10 |
| F1.257.3 | 2.70E+05 | 1.13E−04 | 4.17E−10 |
| F1.60.9 | 3.01E+05 | 7.64E−05 | 2.54E−10 |
| F1.245.2 | 2.60E+06 | 1.19E−03 | 4.58E−10 |
| F1.17.1 | 2.41E+05 | 4.98E−04 | 2.07E−09 |
| F1.77.9 | 1.90E+05 | 3.59E−04 | 1.89E−09 |
| F1.216.2 | 3.73E+04 | 9.44E−04 | 2.53E−08 |
| F1.161.7 | 2.01E+05 | 1.18E−03 | 5.88E−09 |
| F1.280.1 | 6.29E+04 | 4.23E−04 | 6.73E−09 |
| F1.267.9 | 5.44E+03 | 2.23E−04 | 4.09E−08 |
| F1.7.6 | 1.20E+05 | 2.03E−04 | 1.70E−09 |
| F1.62.10 | 1.36E+04 | 3.99E−04 | 2.93E−08 |
| F1.192.1 | 2.25E+05 | 2.44E−04 | 1.08E−09 |
| F1.214.8 | 1.51E+05 | 2.42E−03 | 1.60E−08 |
| F1.224.1 | 1.94E+04 | 2.67E−04 | 1.38E−08 |
| F1.273.12 | 1.21E+05 | 3.75E−04 | 3.10E−09 |
| F1.231.15 | 5.45E+04 | 2.44E−03 | 4.48E−08 |
| F1.250.16 | 4.22E+05 | 6.07E−04 | 1.44E−09 |
| F1.120.15 | 1.19E+04 | 2.32E−04 | 1.95E−08 |
| F1.172.13 | 3.06E+05 | 5.39E−03 | 1.76E−08 |
| F1.200.11 | 9.98E+03 | 1.02E−03 | 1.02E−07 |
| F2.205.9 | 2.46E+05 | 3.86E−04 | 1.57E−09 |
| F2.99.1 | 1.05E+05 | 1.62E−04 | 1.55E−09 |
| F2.70.2 | 6.95E+04 | 7.44E−04 | 1.07E−08 |
| F2.127.11 | 2.26E+05 | 6.60E−04 | 2.93E−09 |
| F2.55.1 | 1.27E+05 | 7.42E−05 | 5.86E−10 |
| F2.104.10 | 7.14E+04 | 6.10E−04 | 8.54E−09 |
| F2.42.9 | 6.61E+04 | 6.63E−04 | 1.00E−08 |
| F2.180.16 | 8.45E+03 | 1.55E−04 | 1.84E−08 |
| F2.173.9 | 4.90E+04 | 2.32E−04 | 4.74E−09 |
| F2.121.9 | 4.91E+03 | 3.93E−04 | 8.00E−08 |
| F2.343.16 | 2.04E+04 | 1.01E−01 | 4.97E−06 |
| F2.151.13 | 2.35E+04 | 5.33E−04 | 2.27E−08 |
| HA22 | 6.74E+04 | 8.96E−05 | 1.33E−09 |
| m971 | 1.87E+05 | 1.31E−02 | 7.02E−08 |

### 7.2 Affinity Assay of Chimeric Antibodies to Rhesus Monkey CD22-ECD-His Protein

According to the method in Example 7.1, the affinity of the chimeric antibodies to rhesus monkey CD22 (ACROBiosystems, catalog number: S12-R52Ha) protein was determined, and the results are shown in Table 20. The highest affinity of the chimeric antibodies to rhesus monkey CD22 can reach 2.04E-09M.

TABLE 20
Affinity of chimeric antibodies to rhesus
monkey CD22 detected by SPR (biacore)

| Designation | ka | kd | KD |
|---|---|---|---|
| of antibody | (1/Ms) | (1/s) | (M) |
| F1.11.7 | 5.94E+04 | 1.11E−03 | 1.87E−08 |
| F1.105.11 | 2.59E+04 | 4.48E−04 | 1.73E−08 |
| F1.17.1 | 1.44E+05 | 5.31E−04 | 3.67E−09 |
| F1.216.2 | 7.49E+04 | 5.38E−04 | 7.18E−09 |
| F1.267.9 | 2.67E+04 | 6.00E−05 | 2.25E−09 |
| F1.192.1 | 6.92E+04 | 1.60E−03 | 2.31E−08 |
| F1.224.1 | 5.23E+04 | 1.07E−04 | 2.04E−09 |
| F1.273.12 | There is binding/the fit is poor | | |
| F1.231.15 | There is binding/the fit is poor | | |
| F1.250.16 | 3.66E+05 | 1.27E−03 | 3.48E−09 |
| F1.120.15 | 4.21E+04 | 6.37E−04 | 1.51E−08 |
| F1.172.13 | 2.00E+05 | 1.38E−03 | 6.90E−09 |
| F1.200.11 | 1.35E+04 | 5.96E−04 | 4.42E−08 |
| F2.205.9 | 1.35E+05 | 1.40E−03 | 1.03E−08 |
| F2.99.1 | 3.76E+04 | 5.45E−04 | 1.45E−08 |
| F2.55.1 | There is binding/the fit is poor | | |
| F2.42.9 | There is binding/the fit is poor | | |
| F2.173.9 | 1.89E+04 | 8.83E−04 | 4.67E−08 |
| F2.121.9 | There is binding/the fit is poor | | |
| F2.151.13 | 1.92E+04 | 5.28E−04 | 2.75E−08 |
| HA22 | 6.86E+04 | 2.34E−04 | 3.41E−09 |

Remark: “Poor fit” indicates that EC50 value could not be calculated.

### EXAMPLE 8

### Identification Of Antigen-Binding Region of Antibody

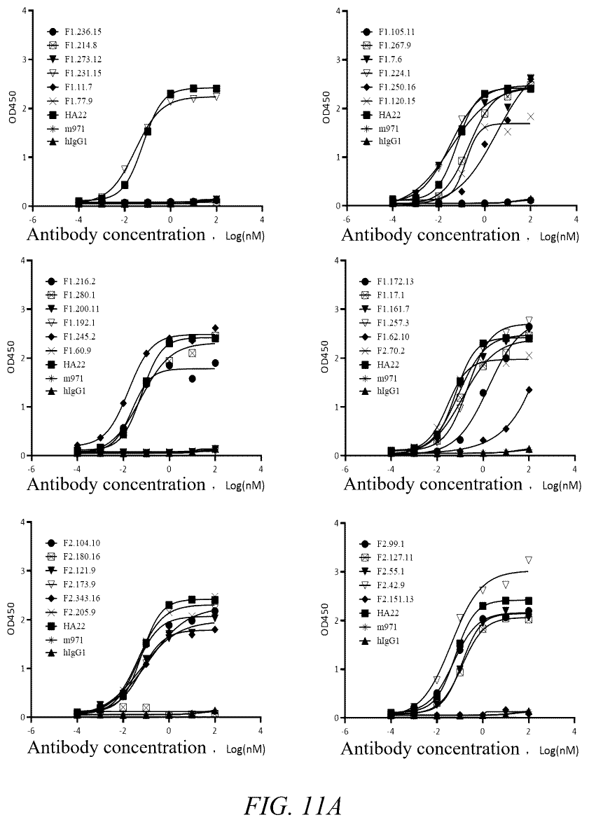

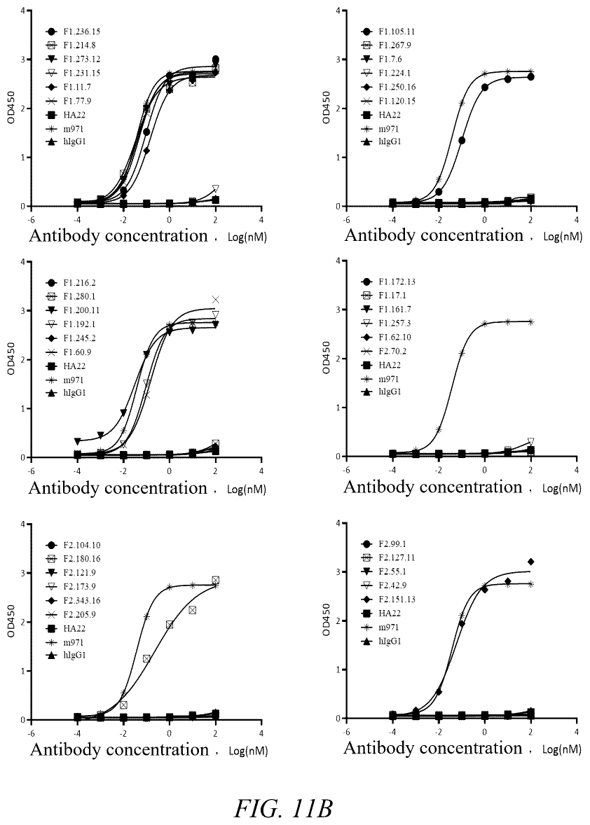

The CD22 protein has 7 IgG-like domains outside the cell, in which domain 1 is located at the farthest end from the membrane and domain 7 is located at the nearest end from the membrane, the antigen-binding epitopes of HA22 and hL22 are located in domain2-3, and the antigen-binding epitope of m971 is located in domain5-7. In order to identify the antigen-binding epitope distribution of the chimeric antibodies, according to the ELISA method in Example 5.1, human CD22-domain1-4-His (distal end of membrane) and human CD22 domain5-7-His (proximal end of membrane) were used for coating, respectively. The chimeric antibodies were classified into the type of distal end of membrane and the type of proximal end of membrane, as shown in FIGS. 11A-11B and Table 21, the antibodies can be divided into three categories: the first category does not bind to CD22 domain5-7, has equivalent binding activity to CD22 domain1-4 and CD22 full-length ECD and has a binding epitope located in domain1-4, such as F1.231.15; the second category does not bind to CD22 domain1-4, has equivalent binding activity to CD22 domain5-7 and CD22 full-length ECD and has a binding epitope located in domain5-7, such as F1.236.15; the third category does not bind to CD22 domain5-7 and has lower binding activity to CD22 domain1-4 than CD22 full-length ECD, such as F1.250.16, F1.172.13 and F1.62.10.

TABLE 21
Classification of the chimeric antibodies by ELISA
method according to epitopes at the distal end of
membrane and epitopes at the proximal end of membrane

| | |
|---|---|
| Designation | Binding region |

| of antibody | Domain 1-4 | Domain5-7 |
|---|---|---|
| F1.236.15 | − | + |
| F1.214.8 | − | + |
| F1.273.12 | − | + |
| F1.231.15 | + | − |
| F1.11.7 | − | + |
| F1.77.9 | − | + |
| F1.105.11 | − | + |
| F1.267.9 | + | − |
| F1.7.6 | + | − |
| F1.224.1 | + | − |
| F1.250.16 | Weak binding | − |
| F1.120.15 | + | − |
| F1.216.2 | + | − |
| F1.280.1 | + | − |
| F1.200.11 | − | + |
| F1.192.1 | − | + |
| F1.245.2 | + | − |
| F1.60.9 | − | + |
| F1.172.13 | Weak binding | − |
| F1.17.1 | + | − |
| F1.161.7 | + | − |
| F1.257.3 | + | − |
| F1.62.10 | Weak binding | − |
| F2.70.2 | + | − |
| F2.104.10 | + | − |
| F2.180.16 | − | + |
| F2.121.9 | + | − |
| F2.173.9 | + | − |
| F2.343.16 | + | − |
| F2.205.9 | + | − |
| F2.99.1 | + | − |
| F2.127.11 | + | − |
| F2.55.1 | + | − |
| F2.42.9 | + | − |
| F2.151.13 | − | + |
| HA22 | + | − |
| m971 | − | + |

“+” indicates that there is binding in this region.
“−” indicates that there is no binding in this region.

1. An isolated antibody or an antigen-binding fragment that specifically binds to human CD22, characterized in that, the antibody or the antigen-binding fragment comprises a combination of heavy chain CDRs and a combination of light chain CDRs:
(1) the combination of the heavy chain CDRs comprises: CDR1-VH, CDR2-VH and CDR3-VH; the CDR1-VH, CDR2-VH and CDR3-VH have any sequence combination selected from the following or a sequence combination with 1, 2, 3 or more amino acid insertions, deletions and/or substitutions compared to the sequence combination:

| | SEQ ID NO. | | |
|---|---|---|---|
| No. | CDR1-VH | CDR2-VH | CDR3-VH |
| VH1 | SEQ ID NO. 83 | SEQ ID NO. 84 | SEQ ID NO. 85 |
| VH2 | SEQ ID NO. 86 | SEQ ID NO. 87 | SEQ ID NO. 88 |
| VH3 | SEQ ID NO. 89 | SEQ ID NO. 90 | SEQ ID NO. 91 |
| VH4 | SEQ ID NO. 101 | SEQ ID NO. 102 | SEQ ID NO. 103 |
| VH5 | SEQ ID NO. 104 | SEQ ID NO. 105 | SEQ ID NO. 106 |
| VH6 | SEQ ID NO. 107 | SEQ ID NO. 108 | SEQ ID NO. 109 |
| VH7 | SEQ ID NO. 119 | SEQ ID NO. 120 | SEQ ID NO. 121 |
| VH8 | SEQ ID NO. 122 | SEQ ID NO. 123 | SEQ ID NO. 124 |
| VH9 | SEQ ID NO. 125 | SEQ ID NO. 126 | SEQ ID NO. 127 |
| VH10 | SEQ ID NO. 137 | SEQ ID NO. 138 | SEQ ID NO. 139 |
| VH11 | SEQ ID NO. 140 | SEQ ID NO. 141 | SEQ ID NO. 142 |
| VH12 | SEQ ID NO. 143 | SEQ ID NO. 144 | SEQ ID NO. 145 |
| VH13 | SEQ ID NO. 155 | SEQ ID NO. 156 | SEQ ID NO. 157 |
| VH14 | SEQ ID NO. 158 | SEQ ID NO. 159 | SEQ ID NO. 160 |
| VH15 | SEQ ID NO. 161 | SEQ ID NO. 162 | SEQ ID NO. 163 |
| VH16 | SEQ ID NO. 173 | SEQ ID NO. 174 | SEQ ID NO. 175 |
| VH17 | SEQ ID NO. 176 | SEQ ID NO. 177 | SEQ ID NO. 178 |
| VH18 | SEQ ID NO. 179 | SEQ ID NO. 180 | SEQ ID NO. 181 |
| VH19 | SEQ ID NO. 191 | SEQ ID NO. 192 | SEQ ID NO. 193 |
| VH20 | SEQ ID NO. 194 | SEQ ID NO. 195 | SEQ ID NO. 196 |
| VH21 | SEQ ID NO. 197 | SEQ ID NO. 198 | SEQ ID NO. 199 |
| VH22 | SEQ ID NO. 209 | SEQ ID NO. 210 | SEQ ID NO. 211 |
| VH23 | SEQ ID NO. 212 | SEQ ID NO. 213 | SEQ ID NO. 214 |
| VH24 | SEQ ID NO. 215 | SEQ ID NO. 216 | SEQ ID NO. 217 |
| VH25 | SEQ ID NO. 227 | SEQ ID NO. 228 | SEQ ID NO. 229 |
| VH26 | SEQ ID NO. 230 | SEQ ID NO. 231 | SEQ ID NO. 232 |
| VH27 | SEQ ID NO. 233 | SEQ ID NO. 234 | SEQ ID NO. 235 |
| VH28 | SEQ ID NO. 245 | SEQ ID NO. 246 | SEQ ID NO. 247 |
| VH29 | SEQ ID NO. 248 | SEQ ID NO. 249 | SEQ ID NO. 250 |
| VH30 | SEQ ID NO. 251 | SEQ ID NO. 252 | SEQ ID NO. 253 |
| VH31 | SEQ ID NO. 263 | SEQ ID NO. 264 | SEQ ID NO. 265 |
| VH32 | SEQ ID NO. 266 | SEQ ID NO. 267 | SEQ ID NO. 268 |
| VH33 | SEQ ID NO. 269 | SEQ ID NO. 270 | SEQ ID NO. 271 |
| VH34 | SEQ ID NO. 281 | SEQ ID NO. 282 | SEQ ID NO. 283 |
| VH35 | SEQ ID NO. 284 | SEQ ID NO. 285 | SEQ ID NO. 286 |
| VH36 | SEQ ID NO. 287 | SEQ ID NO. 288 | SEQ ID NO. 289 |
| VH37 | SEQ ID NO. 299 | SEQ ID NO. 300 | SEQ ID NO. 301 |
| VH38 | SEQ ID NO. 302 | SEQ ID NO. 303 | SEQ ID NO. 304 |
| VH39 | SEQ ID NO. 305 | SEQ ID NO. 306 | SEQ ID NO. 307 |
| VH40 | SEQ ID NO. 317 | SEQ ID NO. 318 | SEQ ID NO. 319 |
| VH41 | SEQ ID NO. 320 | SEQ ID NO. 321 | SEQ ID NO. 322 |
| VH42 | SEQ ID NO. 323 | SEQ ID NO. 324 | SEQ ID NO. 325 |
| VH43 | SEQ ID NO. 335 | SEQ ID NO. 336 | SEQ ID NO. 337 |
| VH44 | SEQ ID NO. 338 | SEQ ID NO. 339 | SEQ ID NO. 340 |
| VH45 | SEQ ID NO. 341 | SEQ ID NO. 342 | SEQ ID NO. 343 |
| VH46 | SEQ ID NO. 353 | SEQ ID NO. 354 | SEQ ID NO. 355 |
| VH47 | SEQ ID NO. 356 | SEQ ID NO. 357 | SEQ ID NO. 358 |
| VH48 | SEQ ID NO. 359 | SEQ ID NO. 360 | SEQ ID NO. 361 |
| VH49 | SEQ ID NO. 371 | SEQ ID NO. 372 | SEQ ID NO. 373 |
| VH50 | SEQ ID NO. 374 | SEQ ID NO. 375 | SEQ ID NO. 376 |
| VH51 | SEQ ID NO. 377 | SEQ ID NO. 378 | SEQ ID NO. 379 |
| VH52 | SEQ ID NO. 389 | SEQ ID NO. 390 | SEQ ID NO. 391 |
| VH53 | SEQ ID NO. 392 | SEQ ID NO. 393 | SEQ ID NO. 394 |
| VH54 | SEQ ID NO. 395 | SEQ ID NO. 396 | SEQ ID NO. 397 |
| VH55 | SEQ ID NO. 407 | SEQ ID NO. 408 | SEQ ID NO. 409 |
| VH56 | SEQ ID NO. 410 | SEQ ID NO. 411 | SEQ ID NO. 412 |
| VH57 | SEQ ID NO. 413 | SEQ ID NO. 414 | SEQ ID NO. 415 |
| VH58 | SEQ ID NO. 425 | SEQ ID NO. 426 | SEQ ID NO. 427 |
| VH59 | SEQ ID NO. 428 | SEQ ID NO. 429 | SEQ ID NO. 430 |
| VH60 | SEQ ID NO. 431 | SEQ ID NO. 432 | SEQ ID NO. 433 |
| VH61 | SEQ ID NO. 443 | SEQ ID NO. 444 | SEQ ID NO. 445 |
| VH62 | SEQ ID NO. 446 | SEQ ID NO. 447 | SEQ ID NO. 448 |
| VH63 | SEQ ID NO. 449 | SEQ ID NO. 450 | SEQ ID NO. 451 |
| VH64 | SEQ ID NO. 461 | SEQ ID NO. 462 | SEQ ID NO. 463 |
| VH65 | SEQ ID NO. 464 | SEQ ID NO. 465 | SEQ ID NO. 466 |
| VH66 | SEQ ID NO. 467 | SEQ ID NO. 468 | SEQ ID NO. 469 |
| VH67 | SEQ ID NO. 479 | SEQ ID NO. 480 | SEQ ID NO. 481 |
| VH68 | SEQ ID NO. 482 | SEQ ID NO. 483 | SEQ ID NO. 484 |
| VH69 | SEQ ID NO. 485 | SEQ ID NO. 486 | SEQ ID NO. 487 |
| VH70 | SEQ ID NO. 497 | SEQ ID NO. 498 | SEQ ID NO. 499 |
| VH71 | SEQ ID NO. 500 | SEQ ID NO. 501 | SEQ ID NO. 502 |
| VH72 | SEQ ID NO. 503 | SEQ ID NO. 504 | SEQ ID NO. 505 |
| VH73 | SEQ ID NO. 515 | SEQ ID NO. 516 | SEQ ID NO. 517 |
| VH74 | SEQ ID NO. 518 | SEQ ID NO. 519 | SEQ ID NO. 520 |
| VH75 | SEQ ID NO. 521 | SEQ ID NO. 522 | SEQ ID NO. 523 |
| VH76 | SEQ ID NO. 533 | SEQ ID NO. 534 | SEQ ID NO. 535 |
| VH77 | SEQ ID NO. 536 | SEQ ID NO. 537 | SEQ ID NO. 538 |
| VH78 | SEQ ID NO. 539 | SEQ ID NO. 540 | SEQ ID NO. 541 |
| VH79 | SEQ ID NO. 551 | SEQ ID NO. 552 | SEQ ID NO. 553 |
| VH80 | SEQ ID NO. 554 | SEQ ID NO. 555 | SEQ ID NO. 556 |
| VH81 | SEQ ID NO. 557 | SEQ ID NO. 558 | SEQ ID NO. 559 |
| VH82 | SEQ ID NO. 569 | SEQ ID NO. 570 | SEQ ID NO. 571 |
| VH83 | SEQ ID NO. 572 | SEQ ID NO. 573 | SEQ ID NO. 574 |
| VH84 | SEQ ID NO. 575 | SEQ ID NO. 576 | SEQ ID NO. 577 |
| VH85 | SEQ ID NO. 587 | SEQ ID NO. 588 | SEQ ID NO. 589 |
| VH86 | SEQ ID NO. 590 | SEQ ID NO. 591 | SEQ ID NO. 592 |
| VH87 | SEQ ID NO. 593 | SEQ ID NO. 594 | SEQ ID NO. 595 |
| VH88 | SEQ ID NO. 605 | SEQ ID NO. 606 | SEQ ID NO. 607 |
| VH89 | SEQ ID NO. 608 | SEQ ID NO. 609 | SEQ ID NO. 610 |
| VH90 | SEQ ID NO. 611 | SEQ ID NO. 612 | SEQ ID NO. 613 |
| VH91 | SEQ ID NO. 623 | SEQ ID NO. 624 | SEQ ID NO. 625 |
| VH92 | SEQ ID NO. 626 | SEQ ID NO. 627 | SEQ ID NO. 628 |
| VH93 | SEQ ID NO. 629 | SEQ ID NO. 630 | SEQ ID NO. 631 |
| VH94 | SEQ ID NO. 641 | SEQ ID NO. 642 | SEQ ID NO. 643 |
| VH95 | SEQ ID NO. 644 | SEQ ID NO. 645 | SEQ ID NO. 646 |
| VH96 | SEQ ID NO. 647 | SEQ ID NO. 648 | SEQ ID NO. 649 |
| VH97 | SEQ ID NO. 659 | SEQ ID NO. 660 | SEQ ID NO. 661 |
| VH98 | SEQ ID NO. 662 | SEQ ID NO. 663 | SEQ ID NO. 664 |
| VH99 | SEQ ID NO. 665 | SEQ ID NO. 666 | SEQ ID NO. 667 |
| VH100 | SEQ ID NO. 677 | SEQ ID NO. 678 | SEQ ID NO. 679 |
| VH101 | SEQ ID NO. 680 | SEQ ID NO. 681 | SEQ ID NO. 682 |
| VH102 | SEQ ID NO. 683 | SEQ ID NO. 684 | SEQ ID NO. 685 |
| VH103 | SEQ ID NO. 695 | SEQ ID NO. 696 | SEQ ID NO. 697 |
| VH104 | SEQ ID NO. 698 | SEQ ID NO. 699 | SEQ ID NO. 700 |
| VH105 | SEQ ID NO. 701 | SEQ ID NO. 702 | SEQ ID NO. 703 |

and,
(2) the combination of the light chain CDRs comprises: CDR1-VL, CDR2-VL, and CDR3-VL, the CDR1-VL, CDR2-VL and CDR3-VL have any sequence combination selected from the following, or a sequence combination with 1, 2, 3 or more amino acid insertions, deletions and/or substitutions compared to the sequence combination:

| | SEQ ID NO. | | |
|---|---|---|---|
| No. | CDR1-VL | CDR2-VL | CDR3-VL |
| VL1 | SEQ ID NO. 92 | SEQ ID NO. 93 | SEQ ID NO. 94 |
| VL2 | SEQ ID NO. 95 | SEQ ID NO. 96 | SEQ ID NO. 97 |
| VL3 | SEQ ID NO. 98 | SEQ ID NO. 99 | SEQ ID NO. 100 |
| VL4 | SEQ ID NO. 110 | SEQ ID NO. 111 | SEQ ID NO. 112 |
| VL5 | SEQ ID NO. 113 | SEQ ID NO. 114 | SEQ ID NO. 115 |
| VL6 | SEQ ID NO. 116 | SEQ ID NO. 117 | SEQ ID NO. 118 |
| VL7 | SEQ ID NO. 128 | SEQ ID NO. 129 | SEQ ID NO. 130 |
| VL8 | SEQ ID NO. 131 | SEQ ID NO. 132 | SEQ ID NO. 133 |
| VL9 | SEQ ID NO. 134 | SEQ ID NO. 135 | SEQ ID NO. 136 |
| VL10 | SEQ ID NO. 146 | SEQ ID NO. 147 | SEQ ID NO. 148 |
| VL11 | SEQ ID NO. 149 | SEQ ID NO. 150 | SEQ ID NO. 151 |
| VL12 | SEQ ID NO. 152 | SEQ ID NO. 153 | SEQ ID NO. 154 |
| VL13 | SEQ ID NO. 164 | SEQ ID NO. 165 | SEQ ID NO. 166 |
| VL14 | SEQ ID NO. 167 | SEQ ID NO. 168 | SEQ ID NO. 169 |
| VL15 | SEQ ID NO. 170 | SEQ ID NO. 171 | SEQ ID NO. 172 |
| VL16 | SEQ ID NO. 182 | SEQ ID NO. 183 | SEQ ID NO. 184 |
| VL17 | SEQ ID NO. 185 | SEQ ID NO. 186 | SEQ ID NO. 187 |
| VL18 | SEQ ID NO. 188 | SEQ ID NO. 189 | SEQ ID NO. 190 |
| VL19 | SEQ ID NO. 200 | SEQ ID NO. 201 | SEQ ID NO. 202 |
| VL20 | SEQ ID NO. 203 | SEQ ID NO. 204 | SEQ ID NO. 205 |
| VL21 | SEQ ID NO. 206 | SEQ ID NO. 207 | SEQ ID NO. 208 |
| VL22 | SEQ ID NO. 218 | SEQ ID NO. 219 | SEQ ID NO. 220 |
| VL23 | SEQ ID NO. 221 | SEQ ID NO. 222 | SEQ ID NO. 223 |
| VL24 | SEQ ID NO. 224 | SEQ ID NO. 225 | SEQ ID NO. 226 |
| VL25 | SEQ ID NO. 236 | SEQ ID NO. 237 | SEQ ID NO. 238 |
| VL26 | SEQ ID NO. 239 | SEQ ID NO. 240 | SEQ ID NO. 241 |
| VL27 | SEQ ID NO. 242 | SEQ ID NO. 243 | SEQ ID NO. 244 |
| VL28 | SEQ ID NO. 254 | SEQ ID NO. 255 | SEQ ID NO. 256 |
| VL29 | SEQ ID NO. 257 | SEQ ID NO. 258 | SEQ ID NO. 259 |
| VL30 | SEQ ID NO. 260 | SEQ ID NO. 261 | SEQ ID NO. 262 |
| VL31 | SEQ ID NO. 272 | SEQ ID NO. 273 | SEQ ID NO. 274 |
| VL32 | SEQ ID NO. 275 | SEQ ID NO. 276 | SEQ ID NO. 277 |
| VL33 | SEQ ID NO. 278 | SEQ ID NO. 279 | SEQ ID NO. 280 |
| VL34 | SEQ ID NO. 290 | SEQ ID NO. 291 | SEQ ID NO. 292 |
| VL35 | SEQ ID NO. 293 | SEQ ID NO. 294 | SEQ ID NO. 295 |
| VL36 | SEQ ID NO. 296 | SEQ ID NO. 297 | SEQ ID NO. 298 |
| VL37 | SEQ ID NO. 308 | SEQ ID NO. 309 | SEQ ID NO. 310 |
| VL38 | SEQ ID NO. 311 | SEQ ID NO. 312 | SEQ ID NO. 313 |
| VL39 | SEQ ID NO. 314 | SEQ ID NO. 315 | SEQ ID NO. 316 |
| VL40 | SEQ ID NO. 326 | SEQ ID NO. 327 | SEQ ID NO. 328 |
| VL41 | SEQ ID NO. 329 | SEQ ID NO. 330 | SEQ ID NO. 331 |
| VL42 | SEQ ID NO. 332 | SEQ ID NO. 333 | SEQ ID NO. 334 |
| VL43 | SEQ ID NO. 344 | SEQ ID NO. 345 | SEQ ID NO. 346 |
| VL44 | SEQ ID NO. 347 | SEQ ID NO. 348 | SEQ ID NO. 349 |
| VL45 | SEQ ID NO. 350 | SEQ ID NO. 351 | SEQ ID NO. 352 |
| VL46 | SEQ ID NO. 362 | SEQ ID NO. 363 | SEQ ID NO. 364 |
| VL47 | SEQ ID NO. 365 | SEQ ID NO. 366 | SEQ ID NO. 367 |
| VL48 | SEQ ID NO. 368 | SEQ ID NO. 369 | SEQ ID NO. 370 |
| VL49 | SEQ ID NO. 380 | SEQ ID NO. 381 | SEQ ID NO. 382 |
| VL50 | SEQ ID NO. 383 | SEQ ID NO. 384 | SEQ ID NO. 385 |
| VL51 | SEQ ID NO. 386 | SEQ ID NO. 387 | SEQ ID NO. 388 |
| VL52 | SEQ ID NO. 398 | SEQ ID NO. 399 | SEQ ID NO. 400 |
| VL53 | SEQ ID NO. 401 | SEQ ID NO. 402 | SEQ ID NO. 403 |
| VL54 | SEQ ID NO. 404 | SEQ ID NO. 405 | SEQ ID NO. 406 |
| VL55 | SEQ ID NO. 416 | SEQ ID NO. 417 | SEQ ID NO. 418 |
| VL56 | SEQ ID NO. 419 | SEQ ID NO. 420 | SEQ ID NO. 421 |
| VL57 | SEQ ID NO. 422 | SEQ ID NO. 423 | SEQ ID NO. 424 |
| VL58 | SEQ ID NO. 434 | SEQ ID NO. 435 | SEQ ID NO. 436 |
| VL59 | SEQ ID NO. 437 | SEQ ID NO. 438 | SEQ ID NO. 439 |
| VL60 | SEQ ID NO. 440 | SEQ ID NO. 441 | SEQ ID NO. 442 |
| VL61 | SEQ ID NO. 452 | SEQ ID NO. 453 | SEQ ID NO. 454 |
| VL62 | SEQ ID NO. 455 | SEQ ID NO. 456 | SEQ ID NO. 457 |
| VL63 | SEQ ID NO. 458 | SEQ ID NO. 459 | SEQ ID NO. 460 |
| VL64 | SEQ ID NO. 470 | SEQ ID NO. 471 | SEQ ID NO. 472 |
| VL65 | SEQ ID NO. 473 | SEQ ID NO. 474 | SEQ ID NO. 475 |
| VL66 | SEQ ID NO. 476 | SEQ ID NO. 477 | SEQ ID NO. 478 |
| VL67 | SEQ ID NO. 488 | SEQ ID NO. 489 | SEQ ID NO. 490 |
| VL68 | SEQ ID NO. 491 | SEQ ID NO. 492 | SEQ ID NO. 493 |
| VL69 | SEQ ID NO. 494 | SEQ ID NO. 495 | SEQ ID NO. 496 |
| VL70 | SEQ ID NO. 506 | SEQ ID NO. 507 | SEQ ID NO. 508 |
| VL71 | SEQ ID NO. 509 | SEQ ID NO. 510 | SEQ ID NO. 511 |
| VL72 | SEQ ID NO. 512 | SEQ ID NO. 513 | SEQ ID NO. 514 |
| VL73 | SEQ ID NO. 524 | SEQ ID NO. 525 | SEQ ID NO. 526 |
| VL74 | SEQ ID NO. 527 | SEQ ID NO. 528 | SEQ ID NO. 529 |
| VL75 | SEQ ID NO. 530 | SEQ ID NO. 531 | SEQ ID NO. 532 |
| VL76 | SEQ ID NO. 542 | SEQ ID NO. 543 | SEQ ID NO. 544 |
| VL77 | SEQ ID NO. 545 | SEQ ID NO. 546 | SEQ ID NO. 547 |
| VL78 | SEQ ID NO. 548 | SEQ ID NO. 549 | SEQ ID NO. 550 |
| VL79 | SEQ ID NO. 560 | SEQ ID NO. 561 | SEQ ID NO. 562 |
| VL80 | SEQ ID NO. 563 | SEQ ID NO. 564 | SEQ ID NO. 565 |
| VL81 | SEQ ID NO. 566 | SEQ ID NO. 567 | SEQ ID NO. 568 |
| VL82 | SEQ ID NO. 578 | SEQ ID NO. 579 | SEQ ID NO. 580 |
| VL83 | SEQ ID NO. 581 | SEQ ID NO. 582 | SEQ ID NO. 583 |
| VL84 | SEQ ID NO. 584 | SEQ ID NO. 585 | SEQ ID NO. 586 |
| VL85 | SEQ ID NO. 596 | SEQ ID NO. 597 | SEQ ID NO. 598 |
| VL86 | SEQ ID NO. 599 | SEQ ID NO. 600 | SEQ ID NO. 601 |
| VL87 | SEQ ID NO. 602 | SEQ ID NO. 603 | SEQ ID NO. 604 |
| VL88 | SEQ ID NO. 614 | SEQ ID NO. 615 | SEQ ID NO. 616 |
| VL89 | SEQ ID NO. 617 | SEQ ID NO. 618 | SEQ ID NO. 619 |
| VL90 | SEQ ID NO. 620 | SEQ ID NO. 621 | SEQ ID NO. 622 |
| VL91 | SEQ ID NO. 632 | SEQ ID NO. 633 | SEQ ID NO. 634 |
| VL92 | SEQ ID NO. 635 | SEQ ID NO. 636 | SEQ ID NO. 637 |
| VL93 | SEQ ID NO. 638 | SEQ ID NO. 639 | SEQ ID NO. 640 |
| VL94 | SEQ ID NO. 650 | SEQ ID NO. 651 | SEQ ID NO. 652 |
| VL95 | SEQ ID NO. 653 | SEQ ID NO. 654 | SEQ ID NO. 655 |
| VL96 | SEQ ID NO. 656 | SEQ ID NO. 657 | SEQ ID NO. 658 |
| VL97 | SEQ ID NO. 668 | SEQ ID NO. 669 | SEQ ID NO. 670 |
| VL98 | SEQ ID NO. 671 | SEQ ID NO. 672 | SEQ ID NO. 673 |
| VL99 | SEQ ID NO. 674 | SEQ ID NO. 675 | SEQ ID NO. 676 |
| VL100 | SEQ ID NO. 686 | SEQ ID NO. 687 | SEQ ID NO. 688 |
| VL101 | SEQ ID NO. 689 | SEQ ID NO. 690 | SEQ ID NO. 691 |
| VL102 | SEQ ID NO. 692 | SEQ ID NO. 693 | SEQ ID NO. 694 |
| VL103 | SEQ ID NO. 704 | SEQ ID NO. 705 | SEQ ID NO. 706 |
| VL104 | SEQ ID NO. 707 | SEQ ID NO. 708 | SEQ ID NO. 709 |
| VL105 | SEQ ID NO. 710 | SEQ ID NO. 711 | SEQ ID NO. 712 |

each CDR1-VH, CDR2-VH, CDR3-VH, CDR1-VL, CDR2-VL and CDR3-VL is coded according to the prevailing analysis methods of KABAT, Chothia or IMGT.

2. The antibody or the antigen-binding fragment of claim 1, characterized in that, the antibody or the antigen-binding fragment comprises a combination of a heavy chain CDR and a light chain CDR selected from: VH1+VL1, VH2+VL2, VH3+VL3, VH4+VL4, VH5+VL5, VH6+VL6, VH7+VL7, VH8+VL8, VH9+VL9, VH10+VL10, VH11+VL11, VH12+VL12, VH13+VL13, VH14+VL14, VH15+VL15, VH16+VL16, VH17+VL17, VH18+VL18, VH19+VL19, VH20+VL20, VH21+VL21, VH22+VL22, VH23+VL23, VH24+VL24, VH25+VL25, VH26+VL26, VH27+VL27, VH28+VL28, VH29+VL29, VH30+VL30, VH31+VL31, VH32+VL32, VH33+VL33, VH34+VL34, VH35+VL35, VH36+VL36, VH37+VL37, VH38+VL38, VH39+VL39, VH40+VL40, VH41+VL41, VH42+VL42, VH43+VL43, VH44+VL44, VH45+VL45, VH46+VL46, VH47+VL47, VH48+VL48, VH49+VL49, VH50+VL50, VH51+VL5 1, VH52+VL52, VH53+VL53, VH54+VL54, VH55+VL55, VH56+VL56, VH57+VL57, VH58+VL58, VH59+VL59, VH60+VL60, VH61+VL61, VH62+VL62, VH63+VL63, VH64+VL64, VH65+VL65, VH66+VL66, VH67+VL67, VH68+VL68, VH69+VL69, VH70+VL70, VH71+VL71, VH72+VL72, VH73+VL73, VH74+VL74, VH75+VL75, VH76+VL76, VH77+VL77, VH78+VL78, VH79+VL79, VH80+VL80, VH81+VL81, VH82+VL82, VH83+VL83, VH84+VL84, VH85+VL85, VH86+VL86, VH87+VL87, VH88+VL88, VH89+VL89, VH90+VL90, VH91+VL91, VH92+VL92, VH93+VL93, VH94+VU4, VH95+VL95, VH96+VL96, VH97+VL97, VH98+VL98, VH99+VL99, VH100+VL100, VH101+VL101, VH102+VL102, VH103+VL103, VH104+VL104, or VH105+VL105, and a combination of CDRs with insertions, deletions and/or substitutions of 1, 2, 3 or more amino acids compared to the sequence of the heavy chain CDR and the light chain CDR in these combinations.

3. The antibody or the antigen-binding fragment of claim 1, characterized in that, the antibody or the antigen-binding fragment comprises:
(1) a heavy chain variable region having a sequence as shown in SEQ ID NO: 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, or 81; a light chain variable region having a sequence as shown in SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, or 82;
(2) an amino acid sequence having at least 90% identity, preferably at least 91%, 92%, 93%, 94%, 95%, 96%, 97%, 98% or 99% identity with the sequence shown in (1) above; or, (3) a framework region of the antibody or the antigen-binding fragment having at least 90% identity, preferably at least 91%, 92%, 93%, 94%, 95%, 96%, 97%, 98%, 99% identity with the framework region of the amino acid sequence as shown in (1) above.

4. The antibody or the antigen-binding fragment of claim 1, characterized in that, the dissociation constant (1(D) of the antibody or the antigen-binding fragment binding to human CD22 is no more than 10⁻⁶ M, and the dissociation constant (KD) of the antibody or the antigen-binding fragment binding to rhesus monkey CD22 (KD) is no more than 10⁻⁸ M;
or, optionally, the antibody or the antigen-binding fragment binds or does not bind to monkey CD22;
optionally, the antibody or the antigen-binding fragment binds to or does not bind to murine CD22.

5. The antibody or the antigen-binding fragment of claim 1, characterized in that, the antibody or the antigen-binding fragment is:
(1) a chimeric antibody or a fragment thereof;
(2) a humanized antibody or a fragment thereof;
(3) a fully human antibody or a fragment thereof;
preferably, the antibody or the antigen-binding fragment is selected from a monoclonal antibody, a polyclonal antibody, a natural antibody, an engineered antibody, a monospecific antibody, a multispecific antibody (for example, a bispecific antibody), a monovalent antibody, a multivalent antibody, a full-length antibody, an antibody fragment, a naked antibody, a conjugated antibody, a humanized antibody, a fully human antibody, Fab, Fab′, F(ab′)2, Fd, Fv, scFv, a diabody or a single domain antibody.

6. The antibody or the antigen-binding fragment of claim 1, characterized in that, the antibody comprises a sequence of the constant region of any one of human or murine antibody IgG1, IgG2, IgG3, IgG4, IgA, IgM, IgE or IgD; preferably, comprises a sequence of the constant region of human or murine antibody IgG1, IgG2, IgG3 or IgG4.

7. The antibody or the antigen-binding fragment of claim 1, characterized in that, the antigen-binding fragment is selected from one or more of F(ab)2, Fab′, Fab, Fv, scFv, a bispecific antibody, a nanobody and an antibody minimum recognition unit.

8. The antibody or the antigen-binding fragment of claim 1, characterized in that, the antibody or the antigen-binding fragment is further coupled with a therapeutic agent or a tracer; preferably, the therapeutic agent is selected from a radioisotope, a chemotherapeutic agent or an immunomodulator, and the tracer is selected from a radiological contrast agent, a paramagnetic ion, a metal, a fluorescent label, a chemiluminescence label, a ultrasound contrast agent or a photosensitizer.

9. A multispecific antigen-binding molecule, characterized in that, the multispecific antigen-binding molecule comprises a first antigen-binding module and a second antigen-binding module, the first antigen-binding module comprises the antibody or the antigen-binding fragment of claim 1, the second antigen-binding module specifically binds to other antigens than CD22 or binds to a CD22 epitope different from the first antigen-binding module;
preferably, the other antigens are selected from CD3, CD16, CD16A, CD4, CD5, CD8, CD14, CD15, CD19, CD20, CD21, CD23, CD25, CD33, CD37, CD38, CD40, CD40L, CD46, CD52, CD54 , CD66(a-d), CD74, CD80, CD126, CD138, B7, MUC, Ia, HLA-DR, tenascin, VEGF, P1GF, ED-B fibronectin, oncogene products, IL-2, IL-6, TRAIL-R1 or TRAIL-R2;
preferably, the multispecific antibody is a bispecific antibody, a trispecific antibody or a tetraspecific antibody.

10. A chimeric antigen receptor (CAR), characterized in that, the chimeric antigen receptor at least comprises an extracellular antigen-binding domain, a transmembrane domain and an intracellular signaling domain, and the extracellular antigen-binding domain comprises the CD22 antibody or the antigen-binding fragment of claim 1.

11. An immune effector cell, characterized in that, the immune effector cell comprises the chimeric antigen receptor of claim 10 or comprises a nucleic acid fragment encoding the chimeric antigen receptor of claim 10;
preferably, the immune effector cell is selected from a T cell, a NK cell (a natural killer cell), a NKT cell (a natural killer T cell), a monocyte, a macrophage, a dendritic cell or a mast cell; the T cell may be selected from an inflammatory T cell, a cytotoxic T cell, a regulatory T cell (Treg) or a helper T cell;
preferably, the immune effector cell is an allogeneic immune effector cell or an autologous immune cell.

12. An isolated nucleic acid molecule, characterized in that, the nucleic acid molecule encodes the antibody or the antigen-binding fragment of claim 1.

13. An expression vector, characterized in that the expression vector comprises the isolated nucleic acid molecule of claim 12.

14. An isolated host cell, characterized in that the isolated host cell comprises the isolated nucleic acid molecule of claim 12;
preferably, the host cell is a eukaryotic cell or a prokaryotic cell; more preferably, the host cell is derived from a mammalian cell, a yeast cell, an insect cell, Escherichia coli and/or Bacillus subtilis; more preferably, the host cell is selected from an HEK293E cell or a CHO cell.

15. A method for preparing the antibody or the antigen-binding fragment of claim 1, characterized in that, a host cell is cultured or cultured under appropriate conditions, and the antibody or the antigen-binding fragment is isolated, and
wherein the host cell is an isolated host cell comprising an isolated nucleic acid molecule, and the nucleic acid molecule encodes the antibody or the antigen-binding fragment.

16. A method for preparing an immune effector cell, characterized in that, the method comprises introducing a nucleic acid fragment encoding the CAR of claim 10 into the immune effector cell, optionally, the method further comprises enabling the immune effector cell to express the CAR.

17. A pharmaceutical composition, wherein the composition comprises the antibody or the antigen-binding fragment of claim 1; preferably, the composition further comprises a pharmaceutically acceptable carrier, diluent or adjuvant; preferably, the pharmaceutical composition further comprises an additional antineoplastic agent.

18. (canceled)

19. A method for preventing and/or treating a B-cell disease, characterized in that, the method comprises administering an effective amount of the antibody or the antigen-binding fragment of claim 1 to a patient in need thereof; the B cell disease is preferably a tumor or an autoimmune disease;
preferably, the tumor is selected from lymphoma or leukemia, more preferably, the lymphoma or leukemia is selected from B-cell lymphoma, non-Hodgkin's lymphoma, mantle cell lymphoma, follicular lymphoma, marginal zone lymphoma, primary mediastinal B-cell lymphoma, diffuse large B-cell lymphoma, precursor B-cell acute lymphocytic leukemia (pre-B ALL), acute lymphocytic leukemia (ALL), chronic lymphocytic leukemia, multiple myeloma;
preferably, the autoimmune disease is selected from systemic lupus erythematosus (SLE), antiphospholipid antibody syndrome, multiple sclerosis, ulcerative colitis, Crohn's disease, rheumatoid arthritis, Sjogren's syndrome, Guillain-Barre syndrome, myasthenia gravis, large vessel vasculitis, medium vessel vasculitis, polyarteritis nodosa, pemphigus, scleroderma, pulmonary hemorrhage-nephritic syndrome, glomerular nephritis, primary biliary cirrhosis, Graves' disease, membranous nephropathy, autoimmune hepatitis, sprue, Addison's disease, polymyositis/dermatomyositis, monoclonal gamma globulin disease, factor VIII deficiency, cryoglobulinemia, peripheral neuropathy, IgM polyneuropathy, chronic neuropathy, and chronic lymphocytic thyroiditis.

20. (canceled)

21. A kit, characterized in that the kit comprises the antibody or the antigen-binding fragment thereof of claim 1; optionally, the kit further comprises an instruction for use.
